(19) United States
(12) Patent Application Publication
(10) Pub. No.: US 2016/0044913 A1
(43) Pub. Date: Feb. 18, 2016

(54) Compositions and Methods for Delaying Senescence in Cut Flower

(71) Applicant: MONSANTO TECHNOLOGY LLC., St. Louis, MO (US)

(72) Inventors: Jill Deikman, St. Louis, MO (US); Nicholas Wagner, St. Louis, MO (US)

(73) Assignee: Monsanto Technology LLC, St. Louis, MO (US)

(21) Appl. No.: 14/776,583

(22) PCT Filed: Mar. 13, 2014
(86) PCT No.: PCT/US14/26301

§ 371 Date: Sep. 14, 2015

Related U.S. Application Data
(60) Provisional application No. 61/793,020, filed on Mar. 15, 2013.

(51) Int. Cl. A01N 3/02; C12N 15/113

(52) U.S. Cl. CPC A01N 3/02; C12N 15/113; C12N 2320/00

(57) ABSTRACT

Provided are novel compositions methods for use in maintaining the fresh appearance of cut flowers and extending their vase life. In particular, double stranded RNA (dsRNA) molecules that suppress EIN2 expression and extend the vase life of cut flowers is provided.

### PRIORITY CLAIMS AND REFERENCE TO RELATED APPLICATIONS

This application claims priority to U.S. Provisional Patent Application No. 61/793,020, filed on Mar. 15, 2013, which is incorporated herein by reference in its entirety.

### INCORPORATION OF SEQUENCE LISTING

A sequence listing is provided herewith as a part of this PCT patent application via the USPTO's EFS system in the file named “59231_Seq_Listing.txt” which is 184,000 bytes in size (measured in MS-Windows®), was created on Mar. 10, 2014, and is incorporated herein by reference in its entirety.

### BACKGROUND

1. Field

The present invention relates generally to compositions and methods for delaying senescence in cut flowers. In particular, the invention relates to double stranded RNA molecules that suppress EIN2 expression and extend the life of cut flowers.

2. Description of the Related Art

Flowers begin to senesce and loose their freshness as soon as they are cut. Extending the vase life of cut flowers would provide significant value to the floral industry, and allow development of a product with appeal to consumers. The hormone ethylene plays an important role in the control of flower senescence for several commercially important species including roses, carnations, petunias, and others. This invention describes a method to suppress flower response to ethylene or to suppress endogenous ethylene biosynthesis to prolong vase life of cut flowers.

Flowers of many species produce ethylene in response to pollination, and this ethylene then serves as a signal to induce senescence and/or abscission of the metabolically expensive petals once they're no longer needed to attract pollinators (Graham, Schippers et al. 2012). Treatment of these flowers with ethylene accelerates flower senescence, and senescence can be delayed by treatment with chemical inhibitors of ethylene action or biosynthesis. Senescence involves active disassembly of cells, and many genes involved in this process have been identified (van Doom and Woltering 2008). The essential role of ethylene in control of carnation flower senescence is highlighted by work showing ethylene is required for expression of most genes up-regulated during floral senescence (Hoeberichts, van Doom et al. 2007).

While flowers can be treated with chemical inhibitors of ethylene perception, such as silver thiosulfate (STS) or I-methyl cyclopropene (I-MCP), to delay senescence, use of these inhibitors has drawbacks. Silver is a heavy metal and incurs a potentially costly hazardous waste stream, restricted use regulations, and/or and outright ban in some regions. 1-MCP is a gas, and can only be used in controlled environments or with specialized packaging. Its effect is temporary, so once the flowers are put into fresh air, ethylene-regulated processes, including flower senescence, can progress (IGee and Clark 2004). Therefore, there is commercial need for a treatment that can delay flower senescence which is more environmentally friendly and easier to apply and control (not a gas).

### SUMMARY

The present embodiments relate to compositions and methods for delaying senescence in a cut flower by suppressing gene expression with a polynucleotide.

Several embodiments relate to compositions and methods for delaying senescence in a cut flower by suppressing EIN2 expression with a polynucleotide.

Several embodiments relate to a method for delaying senescence in a cut flower, comprising topically applying to a plant surface a composition that comprises at least one polynucleotide that comprises at least 18 contiguous nucleotides that are essentially identical or essentially complementary to a EIN2 gene or to a transcript of said gene, wherein the cut flower exhibits delayed senescence that results from suppression of the EIN2 gene. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide is selected from the group consisting of SEQ ID NOs: 8-39. In some embodiments, the polynucleotide comprises at least 18 contiguous nucleotides that are essentially identical or essentially complementary to SEQ ID NO: 4. In some embodiments, the polynucleotide is selected from the group consisting of SEQ ID NOs: 16-21. In other embodiments, the polynucleotide is selected from the group consisting of SEQ ID NOs: 40-45. In some embodiments, the polynucleotide comprises at least 18 contiguous nucleotides that are essentially identical or essentially complementary to SEQ ID NO: 1 or 7. In some embodiments, the polynucleotide comprises at least 18 contiguous nucleotides that are essentially identical or essentially complementary to SEQ ID NO: 46. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Some embodiments relate to a method for delaying senescence in a carnation, comprising topically applying to a plant surface a composition that comprises at least one polynucleotide selected from the group consisting of SEQ ID NO: 8-39, wherein the carnation exhibits delayed senescence that results from suppression of the EIN2 gene. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition further comprises a transfer agent. In some embodiments, the transfer agent comprises an organosilicone preparation. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Some embodiments relate to a method for delaying senescence in a rose, comprising topically applying to a plant surface a composition that comprises at least one polynucleotide comprising at least 18 contiguous nucleotides that are essentially identical or essentially complementary to SEQ ID NO: 46, wherein the rose exhibits delayed senescence that results from suppression of the EIN2 gene. In some embodiments, the polynucleotide is selected from the group consisting of sense ssDNA, sense ssRNA, dsRNA, dsDNA, a double stranded DNA/RNA hybrid, anti-sense ssDNA, or anti-sense ssRNA. In some embodiments, the composition comprises any combination of two or more polynucleotide molecules comprising at least 18 contiguous nucleotides that are essentially identical or essentially complementary to SEQ ID NO: 46. In some embodiments, the composition comprises any combination of 3, 4, 5, 6, 7, 8, 9, or more polynucleotide molecules. In some embodiments, the composition further comprises a transfer agent. In some embodiments, the transfer agent comprises an organosilicone preparation. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Some embodiments relate to a method for delaying senescence in a carnation, comprising topically applying to a plant surface a composition that comprises at least one polynucleotide that comprises at least 18 contiguous nucleotides that are essentially identical or essentially complementary to SEQ ID NO: 4 or to a transcript of SEQ ID NO: 4, wherein the carnation exhibits delayed senescence that results from suppression of the EIN2 gene. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition further comprises a transfer agent. In some embodiments, the transfer agent comprises an organosilicone preparation. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Some embodiments relate to a method for delaying senescence in a rose, comprising topically applying to a plant surface a composition that comprises at least one polynucleotide selected from the group consisting of SEQ ID NO: 40-45, wherein the rose exhibits delayed senescence that results from suppression of the EIN2 gene. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition further comprises a transfer agent. In some embodiments, the transfer agent comprises an organosilicone preparation. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Some embodiments relate to a method for delaying senescence in a rose, comprising topically applying to a plant surface a composition that comprises at least one polynucleotide that comprises at least 18 contiguous nucleotides that are essentially identical or essentially complementary to SEQ ID NOs: 1 or 7 or to a transcript of SEQ ID NOs: 1 or 7, wherein the rose exhibits delayed senescence that results from suppression of the EIN2 gene. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition further comprises a transfer agent. In some embodiments, the transfer agent comprises an organosilicone preparation. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to a composition for extending the vase life of cut flowers, comprising a polynucleotide molecule that comprises at least 18 contiguous nucleotides that are essentially identical or essentially complementary to a EIN2 gene or transcript of said gene, wherein said polynucleotide comprises sense ssDNA, sense ssRNA, dsRNA, dsDNA, a double stranded DNA/RNA hybrid, anti-sense ssDNA, or anti-sense ssRNA. In some embodiments, the composition further comprises a transfer agent. In some embodiments, the transfer agent comprises an organosilicone preparation. In some embodiments, the composition is applied to a cut or exposed surface of the flower stem. In some embodiments, the composition is applied to vase water. In some embodiments, the composition further comprises one or more of nutrients, water uptake stimulants, and chemical preservatives. In some embodiments, the polynucleotide is selected from the group consisting of SEQ ID NO: 8-39. In some embodiments, the polynucleotide comprises at least 18 contiguous nucleotides that are essentially identical or essentially complementary to SEQ ID NO:4. In some embodiments, the polynucleotide is selected from the group consisting of SEQ ID NO: 40-45. In some embodiments, the polynucleotide comprises at least 18 contiguous nucleotides that are essentially identical or essentially complementary to SEQ ID NO: 1 or 7. In some embodiments, the composition further comprises any combination of two or more polynucleotide molecules. In some embodiments, the composition comprises any combination of 3, 4, 5, 6, 7, 8, 9, or more polynucleotide molecules.

Several embodiments relate to a kit comprising one or more cut flowers and a composition for extending the vase life of cut flowers, comprising: a polynucleotide molecule that comprises at least 18 contiguous nucleotides that are essentially identical or essentially complementary to a EIN2 gene or transcript of said gene, wherein said polynucleotide comprises sense ssDNA, sense ssRNA, dsRNA, dsDNA, a double stranded DNA/RNA hybrid, anti-sense ssDNA, or anti-sense ssRNA. In some embodiments, the composition for extending the vase life of cut flowers further comprises a transfer agent, wherein the transfer agent comprises an organosilicone preparation. In some embodiments, the polynucleotide is selected from the group consisting of SEQ ID NO: 8-39. In some embodiments, the polynucleotide comprises at least 18 contiguous nucleotides that are essentially identical or essentially complementary to SEQ ID NO:4. In some embodiments, the polynucleotide is selected from the group consisting of SEQ ID NO: 40-45. In some embodiments, the polynucleotide comprises at least 18 contiguous nucleotides that are essentially identical or essentially complementary to SEQ ID NO: 1 or 7. In some embodiments, the cut flower is a carnation. In some embodiments, the gene or the transcript is a carnation EIN2 gene or transcript. In some embodiments, the polynucleotide molecule is selected from the group consisting SEQ ID NO: 8-39. In some embodiments, the polynucleotide comprises at least 18 contiguous nucleotides that are essentially identical or essentially complementary to SEQ ID NO: 4. In some embodiments, the cut flower is a rose. In some embodiments, the gene or the transcript is a rose EIN2 gene or transcript. In some embodiments, the polynucleotide molecule is selected from the group consisting SEQ ID NO: 40-45. In some embodiments, the polynucleotide comprises at least 18 contiguous nucleotides that are essentially identical or essentially complementary to SEQ ID NOs: 1 or 7. In some embodiments, the composition for extending the vase life of cut flowers further comprises any combination of two or more polynucleotide molecules. In some embodiments, the composition comprises any combination of 3, 4, 5, 6, 7, 8, 9, or more polynucleotide molecules. In some embodiments, the composition for extending the vase life of cut flowers further comprises a transfer agent, nutrients, water uptake stimulants, and chemical preservatives.

Several embodiments relate to compositions and methods for delaying senescence in a cut flower by suppressing EIL1 expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a EIL1 gene or to a transcript of said gene, wherein the cut flower exhibits delayed senescence that results from suppression of the EIL1 gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 47. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing Ethylene-Insensitive 3 expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a Ethylene-Insensitive 3 gene or to a transcript of said gene, wherein the cut flower exhibits delayed senescence that results from suppression of the Ethylene-Insensitive 3 gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 48. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing EIL2 expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a EIL2 gene or to a transcript of said gene, wherein the cut flower exhibits delayed senescence that results from suppression of the EIL2 gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 49 or 50. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing EIL1 expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a EIL1 gene or to a transcript of said gene, wherein the cut flower exhibits delayed senescence that results from suppression of the EIL1 gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 47. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing ACS expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a ACS gene or to a transcript of said gene, wherein the cut flower exhibits delayed senescence that results from suppression of the ACS gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 51, 52, 53 or 54. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing 1-Aminocyclopropane-1-Carboxylate Oxidase expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a 1-Aminocyclopropane-1-Carboxylate Oxidase gene or to a transcript of said gene, wherein the cut flower exhibits delayed senescence that results from suppression of the 1-Aminocyclopropane-1-Carboxylate Oxidase gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 55 or 56. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing Rh-EIN3-1 expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a Rh-EIN3-1 gene or to a transcript of said gene, wherein the cut flower exhibits delayed senescence that results from suppression of the Rh-EIN3-1 gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 57. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing Rh-EIN3-2 expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a Rh-EIN3-2 gene or to a transcript of said gene, wherein the cut flower exhibits delayed senescence that results from suppression of the Rh-EIN3-2 gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 58. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing EIN3-like gene expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a EIN3-like gene or to a transcript of said gene, wherein the cut flower exhibits delayed senescence that results from suppression of the EIN3-like gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 59. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing Rh-ACO1 expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a Rh-ACO1 gene or to a transcript of said gene, wherein the cut flower exhibits delayed senescence that results from suppression of the Rh-ACO1 gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 50. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing ACS5 expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a ACS5 gene or to a transcript of said gene, wherein the cut flower exhibits delayed senescence that results from suppression of the ACS5 gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 61. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing ACS4 expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a ACS4 gene or to a transcript of said gene, wherein the cut flower exhibits delayed senescence that results from suppression of the ACS4 gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 62. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing ACS expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a ACS gene or to a transcript of said gene, wherein the cut flower exhibits delayed senescence that results from suppression of the ACS gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 51, 52, 53 or 54. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing AC3S expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a ACS3 gene or to a transcript of said gene, wherein the cut flower exhibits delayed senescence that results from suppression of the ACS3 gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 63. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing ACS2 expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a ACS2 gene or to a transcript of said gene, wherein the cut flower exhibits delayed senescence that results from suppression of the ACS2 gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 64. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing rh-ACS2 expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a rh-ACS2 gene or to a transcript of said gene, wherein the cut flower exhibits delayed senescence that results from suppression of the rh-ACS2 gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 65. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing rh-ACS1 expression with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a rh-ACS1 gene or to a transcript of said gene, wherein the cut flower exhibits delayed senescence that results from suppression of the rh-ACS1 gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 66. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

Several embodiments relate to compositions and methods for delaying senescence in a cut flower by suppressing expression of a regulatory gene disclosed in Table 9 with a polynucleotide. Several embodiments relate to a method for delaying senescence in a cut flower, comprising topically applying to a plant surface a composition that comprises at least one polynucleotide that comprises at least 18 contiguous nucleotides that are essentially identical or essentially complementary to a gene disclosed in Table 9 or to a transcript of said gene, wherein the cut flower exhibits delayed senescence that results from suppression of said gene. In some embodiments, the flower is a rose. In some embodiments, the composition further comprises a transfer agent. In some embodiments, the transfer agent comprises an organosilicone preparation. In some embodiments, the polynucleotide molecule comprises sense ssDNA, sense ssRNA, dsRNA, dsDNA, a double stranded DNA/RNA hybrid, anti-sense ssDNA, or anti-sense ssRNA. In some embodiments, the polynucleotide comprises at least 18 contiguous nucleotides that are essentially identical or essentially complementary to SEQ ID NO: 67-111. In some embodiments, the composition comprises any combination of two or more polynucleotide molecules. In some embodiments, the composition comprises any combination of 3, 4, 5, 6, 7, 8, 9, or more polynucleotide molecules. In some embodiments, the composition is applied to a cut or exposed surface of the flower stem. In some embodiments, the composition is applied to vase water. In some embodiments, the composition further comprises one or more of nutrients, water uptake stimulants, and chemical preservatives.

### BRIEF DESCRIPTION OF THE DRAWINGS

FIG. 1 depicts the stages of carnation flower senescence. 1=ideal open bloom, no physical defects; 2=<10% senescent petals, slight curling; 3=<50% senescent petals; 4=50% or more senescent petals; 5=90% or more; 6=fully desiccated.

FIG. 2 depicts flower senescence scores by a mosaic plot for flowers treated with 0.1 nmol trigger. The frequency distribution of the senescence scores for each treatment are plotted. For visual comparison, the scale on the right depicts the theoretical relative frequency distribution of this subset.

FIG. 3 shows the percent identity comparison for the coding sequences of Rosa hybrida freedom (Freedom rose), Rosa hybrida osiana (Osiana Rose), Prunus persica (Peach), Solanum lycoperscium (Tomato), Petunia hybrida (Petunia), Arabidopsis thaliana (Arabidopsis), and Dianthus caryophyllus (Carnation).

FIG. 4 shows an alignment of the coding sequences of Rosa hybrida freedom (Freedom rose), Rosa hybrida osiana (Osiana Rose), Prunus persica (Peach), Solanum lycoperscium (Tomato), Petunia hybrida (Petunia), Arabidopsis thaliana (Arabidopsis), and Dianthus caryophyllus (Carnation).

### DETAILED DESCRIPTION

The following is a detailed description of the invention provided to aid those skilled in the art in practicing the present invention. Those of ordinary skill in the art may make modifications and variations in the embodiments described herein without departing from the spirit or scope of the present invention.

### A. DEFINITIONS

Unless otherwise defined, all technical and scientific terms used herein have the same meaning as commonly understood by one of ordinary skill in the art. Where a term is provided in the singular, the plural of that term is also contemplated unless otherwise indicated.

In the description that follows, a number of terms are used extensively. The following definitions are provided to facilitate understanding of the present embodiments.

As used herein, the terms “DNA,” “DNA molecule,” and “DNA polynucleotide molecule” refer to a single-stranded DNA or double-stranded DNA molecule of genomic or synthetic origin, such as, a polymer of deoxyribonucleotide bases or a DNA polynucleotide molecule.

As used herein, the terms “DNA sequence,” “DNA nucleotide sequence,” and “DNA polynucleotide sequence” refer to the nucleotide sequence of a DNA molecule.

As used herein, the term “gene” refers to any portion of a nucleic acid that provides for expression of a transcript or encodes a transcript. A “gene” thus includes, but is not limited to, a promoter region, 5′ untranslated regions, transcript encoding regions that can include intronic regions, and 3′ untranslated regions.

As used herein, the terms “RNA,” “RNA molecule,” and “RNA polynucleotide molecule” refer to a single-stranded RNA or double-stranded RNA molecule of genomic or synthetic origin, such as, a polymer of ribonucleotide bases that comprise single or double stranded regions.

Unless otherwise stated, nucleotide sequences in the text of this specification are given, when read from left to right, in the 5′ to 3′ direction. The nomenclature used herein is that required by Title 37 of the United States Code of Federal Regulations §1.822 and set forth in the tables in WIPO Standard ST.25 (1998), Appendix 2, Tables 1 and 3.

As used herein, a “plant surface” refers to any exterior portion of a plant. Plant surfaces thus include, but are not limited to, the surfaces of flowers, stems, tubers, fruit, anthers, pollen, leaves, roots, or seeds. A plant surface can be on a portion of a plant that is attached to other portions of a plant or on a portion of a plant that is detached from the plant, for example, the cut end of a flower.

As used herein, the term “cut flower” refers to a flower that has been cut, picked or harvested from a plant.

As used herein, the phrase “polynucleotide is not operably linked to a promoter” refers to a polynucleotide that is not covalently linked to a polynucleotide promoter sequence that is specifically recognized by either a DNA dependent RNA polymerase II protein or by a viral RNA dependent RNA polymerase in such a manner that the polynucleotide will be transcribed by the DNA dependent RNA polymerase II protein or viral RNA dependent RNA polymerase. A polynucleotide that is not operably linked to a promoter can be transcribed by a plant RNA dependent RNA polymerase.

As used herein, a first nucleic-acid sequence is “operably” connected or “linked” with a second nucleic acid sequence when the first nucleic acid sequence is placed in a functional relationship with the second nucleic acid sequence. For instance, a promoter is operably linked to an RNA and/or protein-coding sequence if the promoter provides for transcription or expression of the RNA or coding sequence. Generally, operably linked DNA sequences are contiguous and, where necessary to join two protein-coding regions, are in the same reading frame.

As used herein, the phrase “organosilicone preparation” refers to a liquid comprising one or more organosilicone compounds, wherein the liquid or components contained therein, when combined with a polynucleotide in a composition that is topically applied to a target plant surface, for example the cut end of a flower, enable the polynucleotide to enter a plant cell. Examples of organosilicone preparations include, but are not limited to, preparations marketed under the trade names “Silwet®” or “BREAK-THRU®” and preparations provided in Table 1. In certain embodiments, an organosilicone preparation can enable a polynucleotide to enter a plant cell in a manner permitting a polynucleotide mediated suppression of target gene expression in the plant cell.

As used herein, the phrase “delayed senescence” or “delaying senescence” refer to any measurable delay in the onset or progress of a senescence process, for example a delay in flower yellowing or abscission. In certain embodiments, a delay in a senescence process in a flower or flower part can be determined in a comparison to a control flower or flower part that has not been treated with a composition comprising a polynucleotide. When used in this context, a control flower is a flower that has not undergone treatment with polynucleotide. Such control flowers would include, but are not limited to, untreated flowers or mock treated flowers.

As used herein, a “senescence process” refers to any process whereby any visual, physical, and/or biochemical property of a flower or flower part changes as a result of aging.

As used herein, the phrase “provides for a reduction”, when used in the context of a transcript or a protein in a flower or flower part, refers to any measurable decrease in the level of transcript or protein. In certain embodiments, a reduction of the level of a transcript or protein in a flower or flower part can be determined in a comparison to a control flower or flower part that has not been treated with a composition comprising a polynucleotide. When used in this context, a control flower or flower part is a flower or flower part that has not undergone treatment with polynucleotide. Such control flowers or flower parts would include, but are not limited to, untreated or mock treated flowers and flower parts.

As used herein, the phrase “suppressing expression” or “suppression”, when used in the context of a gene, refers any measurable decrease in the amount and/or activity of a product encoded by the gene. Thus, expression of a gene can be suppressed when there is a reduction in levels of a transcript from the gene, a reduction in levels of a protein encoded by the gene, a reduction in the activity of the transcript from the gene, a reduction in the activity of a protein encoded by the gene, any one of the preceding conditions, or any combination of the preceding conditions. In this context, the activity of a transcript includes, but is not limited to, its ability to be translated into a protein and/or to exert any RNA-mediated biologic or biochemical effect. In this context, the activity of a protein includes, but is not limited to, its ability to exert any protein-mediated biologic or biochemical effect. In certain embodiments, a suppression of gene expression in a flower or flower part can be determined in a comparison of gene product levels or activities in a treated flower to a control flower or flower part that has not been treated with a composition comprising a polynucleotide. When used in this context, a control flower or flower part is a flower or flower part that has not undergone treatment with polynucleotide. Such control flowers or flower parts would include, but are not limited to, untreated or mock treated flowers and flower parts.

As used herein, the term “transcript” corresponds to any RNA that is produced from a gene by the process of transcription. A transcript of a gene can thus comprise a primary transcription product which can contain introns or can comprise a mature RNA that lacks introns.

As used herein, the term “liquid” refers to both homogeneous mixtures such as solutions and non-homogeneous mixtures such as suspensions, colloids, micelles, and emulsions.

### II. OVERVIEW

The ethylene signal transduction pathway is relatively well understood (Lin, Zhong et al. 2009). Here we describe a key ethylene signal transduction pathway gene, the Ethylene Insensitive 2 (EIN2) gene, from two varieties of rose, Rosa hybrida osiana (SEQ ID NO: 1) and Rosa hybrida freedom (SEQ ID NO: 7) and carnation, Dianthus caryophyllus (SEQ ID NO:4). EIN2 is a positive regulator of ethylene signaling and has been placed downstream of the ethylene-receptor complex. The N terminus of EIN2 has homology with NRAMP ion transporters, but its exact function in ethylene signaling is not understood. EIN2 is a good target for gene suppression because it's found as a single or low copy number gene in Arabidopsis and many other species. Transgenic suppression of EIN2 in petunia resulted in a delay in flower senescence (Shibuya et al. 2004).

Several embodiments described herein relate to suppression of ethylene signaling by topical application of polynucleotide molecules containing sequences homologous to EIN2 gene or its family members to cut flowers. The polynucleotide molecules consist of double-stranded RNA (dsRNA) or single-stranded or doublestranded DNA that is complementary to the transcribed regions of EIN2. Alternatively, polynucleotides (ssDNA or dsDNA and/or dsRNA) that correspond to the sense or anti-sense strand of the promoters of the targeted genes can be used. The efficacious polynucleotide molecules can be delivered in the vase solution, so that they are taken up through the cut stem, to prolong flower life. Alternatively, efficacious polynucleotides can be sprayed on entire plants or plant parts before harvest or on cut flowers after harvest to prolong flower life.

Several embodiments described herein relate to suppression of senescence by topical application of polynucleotide molecules containing sequences homologous to a gene described in Table 8 or Table 9 or its family members to cut flowers. The polynucleotide molecules consist of double-stranded RNA (dsRNA) or single-stranded or doublestranded DNA that is complementary to the transcribed regions of a gene described in Table 8 or Table 9. Alternatively, polynucleotides (ssDNA or dsDNA and/or dsRNA) that correspond to the sense or anti-sense strand of the promoters of the targeted genes can be used. The efficacious polynucleotide molecules can be delivered in the vase solution, so that they are taken up through the cut stem, to prolong flower life. Alternatively, efficacious polynucleotides can be sprayed on entire plants or plant parts before harvest or on cut flowers after harvest to prolong flower life.

Provided herein are polynucleotide compositions and methods for suppressing expression of a target EIN2 gene in cut flowers to provide delayed senescence and/or improved appearance. Also provided herein are cut flowers and flower parts having suppressed EIN2 expression, which exhibit delayed senescence and/or improved appearance.

Also provided herein are polynucleotide compositions and methods for suppressing expression of a target gene described in Table 8 or Table 9 in cut flowers to provide delayed senescence and/or improved appearance. Also provided herein are cut flowers and flower parts having suppressed expression of a gene described in Table 8 or Table 9, which exhibit delayed senescence and/or improved appearance.

Compositions as described herein may be topically applied to the surface of a plant, such as to a surface of a stem, leaf, or petal, and may optionally include a transfer agent. The methods and polynucleotide compositions described herein can be applied to various cut flowers and ornamental plants, for example, Achillea, Allium, Alstroemeria, Amaryllis, Anemones, Baby's Breath, Bouvardia, Calendula, Calla Lilies, Campanula, Carnation, Celosia, Cosmos, Chrysanthemum, Craspedia Billy Buttons, Crocus, Daffodils, Dahlias, Delphinium, Echinacea, Fall Aster, Freesia, Gardenias, Gerberas, Gerberas Spider, Germini, Gladiolus, Hyacinth, Hydrangea, Hypericum Berry, Iris, Larkspur, Lavender, Lilies, Lily of the Valley, Lisianthus, Lupine, Mums, Orchids, Peonies, Poppy, Ranunculus, Roses, Scabiosa, Snapdragons, Star of Bethlehem, Stephanotis, Sunflowers, Sweet Peas, Sweet William, Tulips, Zinnia and other ethylene-sensitive flowers. The methods and compositions provided herein can also be applied to the plant from which the cut flower is harvested.

Without being bound by theory, the compositions and methods of the present embodiments are believed to operate through one or more of the several natural cellular pathways involved in RNA-mediated gene suppression as generally described in Brodersen and Voinnet (2006), Trends Genetics, 22:268-280; Tomari and Zamore (2005) Genes & Dev., 19:517-529; Vaucheret (2006) Genes Dev., 20:759-771; Meins et al. (2005) Annu. Rev. Cell Dev. Biol., 21:297-318; and Jones-Rhoades et al. (2006) Annu. Rev. Plant Biol., 57:19-53. RNA-mediated gene suppression generally involves a double-stranded RNA (dsRNA) intermediate that is formed intra-molecularly within a single RNA molecule or inter-molecularly between two RNA molecules. This longer dsRNA intermediate is processed by a ribonuclease of the RNAase III family (Dicer or Dicer-like ribonuclease) to one or more shorter double-stranded RNAs, one strand of which is incorporated into the RNA-induced silencing complex (“RISC”). For example, the siRNA pathway involves the cleavage of a longer double-stranded RNA intermediate to small interfering RNAs (“siRNAs”). The size of siRNAs is believed to range from about 19 to about 25 base pairs, but the most common classes of siRNAs in plants include those containing 21 to 24 base pairs (See, Hamilton et al. (2002) EMBO J., 21:4671-4679).

### Polynucleotides

As used herein, “polynucleotide” refers to a DNA or RNA molecule containing multiple nucleotides and generally refers both to “oligonucleotides” (a polynucleotide molecule of 18-25 nucleotides in length) and longer polynucleotides of 26 or more nucleotides. Embodiments of this invention include compositions including oligonucleotides having a length of 18-25 nucleotides (18-mers, 19-mers, 20-mers, 21-mers, 22-mers, 23-mers, 24-mers, or 25-mers), or medium-length polynucleotides having a length of 26 or more nucleotides (polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), or long polynucleotides having a length greater than about 300 nucleotides (e. g., polynucleotides of between about 300 to about 400 nucleotides, between about 400 to about 500 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, or even greater than about 1000 nucleotides in length, for example up to the entire length of a target gene including coding or non-coding or both coding and non-coding portions of the target gene). Where a polynucleotide is double-stranded, its length can be similarly described in terms of base pairs.

Polynucleotide compositions used in the various embodiments of this invention include compositions including oligonucleotides, polynucleotides, or a mixture of both, including: RNA or DNA or RNA/DNA hybrids or chemically modified oligonucleotides or polynucleotides or a mixture thereof. In certain embodiments, the polynucleotide may be a combination of ribonucleotides and deoxyribonucleotides, for example, synthetic polynucleotides consisting mainly of ribonucleotides but with one or more terminal deoxyribonucleotides or synthetic polynucleotides consisting mainly of deoxyribonucleotides but with one or more terminal dideoxyribonucleotides. In certain embodiments, the polynucleotide includes non-canonical nucleotides such as inosine, thiouridine, or pseudouridine. In certain embodiments, the polynucleotide includes chemically modified nucleotides. Examples of chemically modified oligonucleotides or polynucleotides are well known in the art; see, for example, U.S. Patent Publication 2011/0171287, U.S. Patent Publication 2011/0171176, U.S. Patent Publication 2011/0152353, U.S. Patent Publication 2011/0152346, and U.S. Patent Publication 2011/0160082, which are herein incorporated by reference. Illustrative examples include, but are not limited to, the naturally occurring phosphodiester backbone of an oligonucleotide or polynucleotide which can be partially or completely modified with phosphorothioate, phosphorodithioate, or methylphosphonate internucleotide linkage modifications, modified nucleoside bases or modified sugars can be used in oligonucleotide or polynucleotide synthesis, and oligonucleotides or polynucleotides can be labeled with a fluorescent moiety (e. g., fluorescein or rhodamine) or other label (e. g., biotin).

Polynucleotides can be single- or double-stranded RNA, single- or double-stranded DNA, double-stranded DNA/RNA hybrids, and modified analogues thereof. In certain embodiments of the invention, the polynucleotides that provide single-stranded RNA in the plant cell may be: (a) a single-stranded RNA molecule (ssRNA), (b) a single-stranded RNA molecule that self-hybridizes to form a double-stranded RNA molecule, (c) a double-stranded RNA molecule (dsRNA), (d) a single-stranded DNA molecule (ssDNA), (e) a single-stranded DNA molecule that self-hybridizes to form a double-stranded DNA molecule, (f) a single-stranded DNA molecule including a modified Pol III gene that is transcribed to an RNA molecule, (g) a double-stranded DNA molecule (dsDNA), (h) a double-stranded DNA molecule including a modified Pol III gene that is transcribed to an RNA molecule, and (i) a double-stranded, hybridized RNA/DNA molecule, or combinations thereof. In certain embodiments, these polynucleotides can comprise both ribonucleic acid residues and deoxyribonucleic acid residues. In certain embodiments, these polynucleotides include chemically modified nucleotides or non-canonical nucleotides. In certain embodiments of the methods, the polynucleotides include double-stranded DNA formed by intramolecular hybridization, double-stranded DNA formed by intermolecular hybridization, double-stranded RNA formed by intramolecular hybridization, or double-stranded RNA formed by intermolecular hybridization. In certain embodiments where the polynucleotide is a dsRNA, the anti-sense strand will comprise at least 18 nucleotides that are essentially complementary to the target gene. In certain embodiments the polynucleotides include single-stranded DNA or single-stranded RNA that self-hybridizes to form a hairpin structure having an at least partially double-stranded structure including at least one segment that will hybridize to RNA transcribed from the gene targeted for suppression. Not intending to be bound by any mechanism, it is believed that such polynucleotides are or will produce single-stranded RNA with at least one segment that will hybridize to RNA transcribed from the gene targeted for suppression. In certain embodiments, the polynucleotides can be operably linked to a promoter—generally a promoter functional in a plant, for example, a pol II promoter, a pol III promoter, a pol IV promoter, or a pol V promoter.

Several embodiments relate to polynucleotide molecules designed to modulate expression by inducing regulation or suppression of an endogenous EIN2 gene in a plant and are designed to have a nucleotide sequence essentially identical or essentially complementary to the nucleotide sequence of an endogenous EIN2 gene of a plant or to the sequence of RNA transcribed from an endogenous EIN2 gene of a plant, which can be coding sequence or non-coding sequence. Several embodiments relate to polynucleotide molecules designed to modulate expression by inducing regulation or suppression of an endogenous gene as described in Table 8 or Table 9 in a plant and are designed to have a nucleotide sequence essentially identical or essentially complementary to the nucleotide sequence of a gene as described in Table 8 or Table 9 of a plant or to the sequence of RNA transcribed from an endogenous gene of a plant, which can be coding sequence or non-coding sequence. These effective polynucleotide molecules that modulate expression are referred to herein as “a trigger, or triggers”. By “essentially identical” or “essentially complementary” it is meant that the trigger polynucleotides (or at least one strand of a double-stranded polynucleotide) have sufficient identity or complementarity to the endogenous gene or to the RNA transcribed from the endogenous gene (e.g. the transcript) to suppress expression of the endogenous gene (e.g. to effect a reduction in levels or activity of the gene transcript and/or encoded protein). In certain embodiments, the trigger polynucleotides provided herein can be directed to a transgene present in the plant. Polynucleotides of the methods and compositions provided herein need not have 100 percent identity to a complementarity to the endogenous gene or to the RNA transcribed from the endogenous gene (i.e. the transcript) to suppress expression of the endogenous gene (i.e. to effect a reduction in levels or activity of the gene transcript or encoded protein). Thus, in certain embodiments, the polynucleotide or a portion thereof is designed to be essentially identical to, or essentially complementary to, a sequence of at least 18 or 19 contiguous nucleotides in either the target gene or messenger RNA transcribed from the target gene (e.g. the transcript). In certain embodiments, an “essentially identical” polynucleotide has 100 percent sequence identity or at least about 83, 84, 85, 86, 87, 88, 89, 90, 91, 92, 93, 94, 95, 96, 97, 98, or 99 percent sequence identity when compared to the sequence of 18 or more contiguous nucleotides in either the endogenous target gene or to an RNA transcribed from the target gene (e.g. the transcript). In certain embodiments, an “essentially complementary” polynucleotide has 100 percent sequence complementarity or at least about 83, 84, 85, 86, 87, 88, 89, 90, 91, 92, 93, 94, 95, 96, 97, 98, or 99 percent sequence complementarity when compared to the sequence of 18 or more contiguous nucleotides in either the target gene or RNA transcribed from the target gene.

In certain embodiments, polynucleotides used in the methods and compositions provided herein can be essentially identical or essentially complementary to any of: i) conserved regions of EIN2 genes of both monocot and dicot plants; ii) conserved regions of EIN2 genes of monocot plants; or iii) conserved regions of EIN2 genes of dicot plants. Such polynucleotides that are essentially identical or essentially complementary to such conserved regions can be used to delay senescence and/or improved appearance by suppressing expression of EIN2 genes in various dicot plants.

Polynucleotides containing mismatches to the target gene or transcript can thus be used in certain embodiments of the compositions and methods provided herein. In certain embodiments, a polynucleotide can comprise at least 19 contiguous nucleotides that are essentially identical or essentially complementary to said gene or said transcript or comprises at least 19 contiguous nucleotides that are essentially identical or essentially complementary to the target gene or target gene transcript. In certain embodiments, a polynucleotide of 19 continuous nucleotides that is essentially identical or essentially complementary to the endogenous target gene or to an RNA transcribed from the target gene (e.g. the transcript) can have 1 or 2 mismatches to the target gene or transcript. In certain embodiments, a polynucleotide of 20 or more nucleotides that contains a contiguous 19 nucleotide span of identity or complementarity to the endogenous target gene or to an RNA transcribed from the target gene can have 1 or 2 mismatches to the target gene or transcript. In certain embodiments, a polynucleotide of 21 continuous nucleotides that is essentially identical or essentially complementary to the endogenous target gene or to an RNA transcribed from the target gene (e.g. the transcript) can have 1, 2, or 3 mismatches to the target gene or transcript. In certain embodiments, a polynucleotide of 22 or more nucleotides that contains a contiguous 21 nucleotide span of identity or complementarity to the endogenous target gene or to an RNA transcribed from the target gene can have 1, 2, or 3 mismatches to the target gene or transcript. In designing polynucleotides with mismatches to an endogenous target gene or to an RNA transcribed from the target gene, mismatches of certain types and at certain positions that are more likely to be tolerated can be used. In certain embodiments, mismatches formed between adenine and cytosine or guanosine and uracil residues are used as described by Du et al. Nucleic Acids Research, 2005, Vol. 33, No. 5 1671-1677. In certain embodiments, mismatches in 19 base pair overlap regions can be at the low tolerance positions 5, 7, 8 or 11 (from the 5′ end of a 19 nucleotide target) with well tolerated nucleotide mismatch residues, at medium tolerance positions 3, 4, and 12-17, and/or at the high tolerance nucleotide positions at either end of the region of complementarity (i.e. positions 1, 2, 18, and 19) as described by Du et al. Nucleic Acids Research, 2005, Vol. 33, No. 5 1671-1677. It is further anticipated that tolerated mismatches can be empirically determined in assays where the polynucleotide is applied to the plants via the methods provided herein and the treated plants assayed for suppression of EIN2 gene expression or appearance of delayed senescence and/or improved appearance.

In certain embodiments, polynucleotide molecules are designed to have 100 percent sequence identity with or complementarity to one allele or one family member of a given target EIN2 gene coding or non-coding sequence. Target EIN2 genes include both the EIN2 genes of SEQ ID NOs: 1, 4, 6, and 7, as well as, orthologous EIN2 genes obtainable from other plants. In other embodiments, the polynucleotide molecules are designed to have 100 percent sequence identity with or complementarity to multiple alleles or family members of a given target gene.

In certain embodiments, polynucleotide molecules are designed to have less than 100 percent sequence identity with or complementarity to one allele or one family member of a given target EIN2 gene coding or non-coding sequence, including the EIN2 genes of SEQ ID NOs: 1, 4, 6, and 7 and orthologous EIN2 genes obtainable from other plants. For example, the polynucleotide molecules are designed to have at least 85%, 86%, 87%, 88%, 89%, 90%, 91%, 92%, 93%, 94%, 95%, 96%, 97%, 98% or at least 99% percent sequence identity with or complementarity to one allele or one family member of a given target EIN2 gene coding or non-coding sequence.

In certain embodiments, polynucleotide compositions and methods provided herein typically effect regulation or modulation (e. g., suppression) of gene expression during a period of at least 1 week or longer and typically in systemic fashion. For instance, within days of treating a plant leaf with a polynucleotide composition as described herein, primary and transitive siRNAs can be detected in other leaves lateral to and above the treated leaf and in apical tissue. In certain embodiments, methods of systemically suppressing expression of a gene in a cut flower, the methods comprising treating said cut flower with a composition comprising at least one polynucleotide, wherein said polynucleotide comprises at least 18 or at least 19 contiguous nucleotides that are essentially identical or essentially complementary to a gene or a transcript encoding a gene of the plant from which the flower is harvested are provided, whereby expression of the gene in said cut flower is systemically suppressed in comparison to a control cut flower that has not been treated with the composition. In some embodiments, the composition further comprises a transfer agent.

Compositions used to suppress a target gene can comprise one or more polynucleotides that are essentially identical or essentially complementary to multiple genes, or to multiple segments of one or more genes. In certain embodiments, compositions used to suppress a target gene can comprise one or more polynucleotides that are essentially identical or essentially complementary to multiple consecutive segments of a target gene, multiple non-consecutive segments of a target gene, multiple alleles of a target gene, or multiple target genes from one or more species.

In certain embodiments, the polynucleotide includes two or more copies of a nucleotide sequence (of 18 or more nucleotides) where the copies are arranged in tandem fashion. In another embodiment, the polynucleotide includes two or more copies of a nucleotide sequence (of 18 or more nucleotides) where the copies are arranged in inverted repeat fashion (forming an at least partially self-complementary strand). The polynucleotide can include both tandem and inverted-repeat copies. Whether arranged in tandem or inverted repeat fashion, each copy can be directly contiguous to the next, or pairs of copies can be separated by an optional spacer of one or more nucleotides. The optional spacer can be unrelated sequence (i.e., not essentially identical to or essentially complementary to the copies, nor essentially identical to, or essentially complementary to, a sequence of 18 or more contiguous nucleotides of the endogenous target gene or RNA transcribed from the endogenous target gene). Alternatively the optional spacer can include sequence that is complementary to a segment of the endogenous target gene adjacent to the segment that is targeted by the copies. In certain embodiments, the polynucleotide includes two copies of a nucleotide sequence of between about 20 to about 30 nucleotides, where the two copies are separated by a spacer no longer than the length of the nucleotide sequence.

Tiling

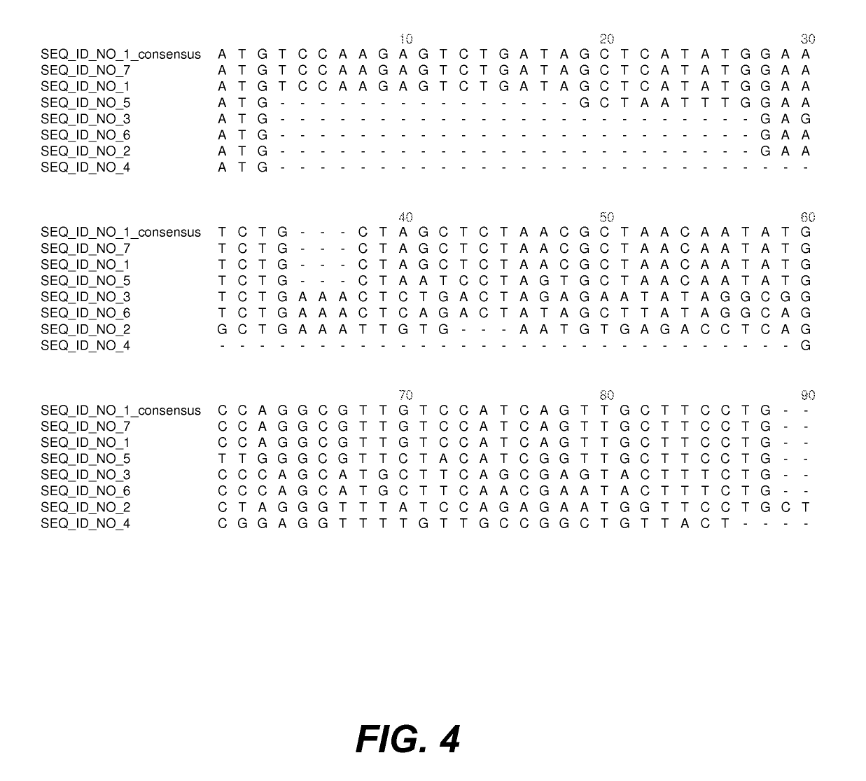

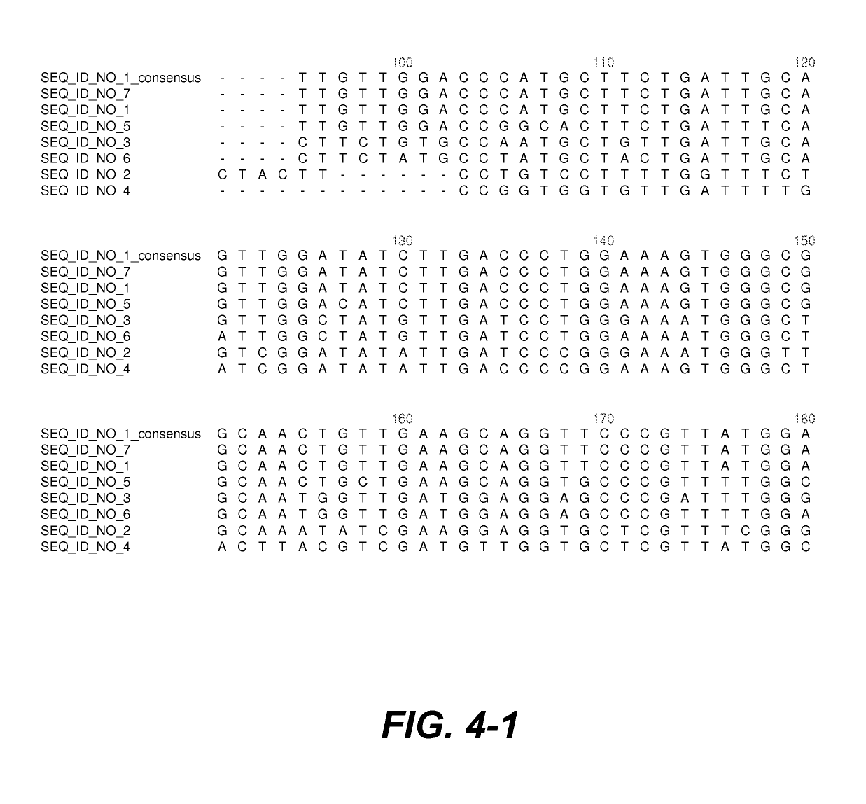

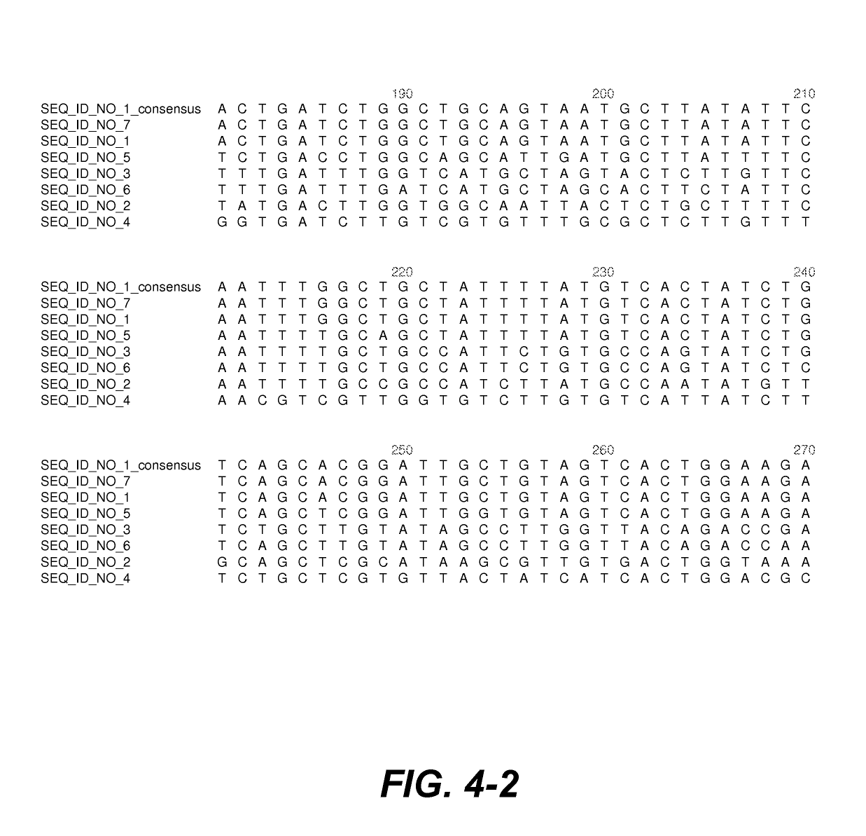

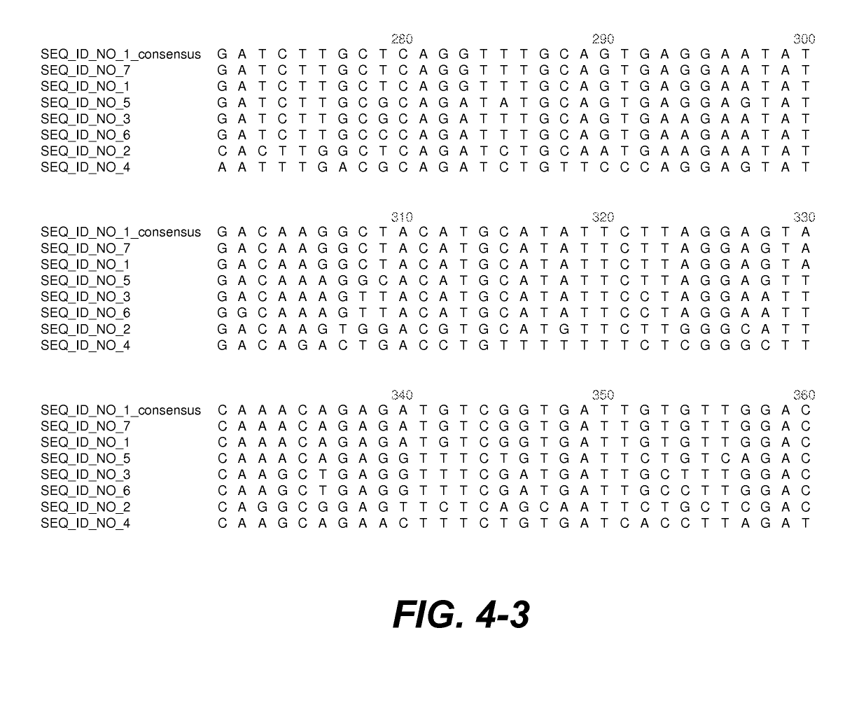

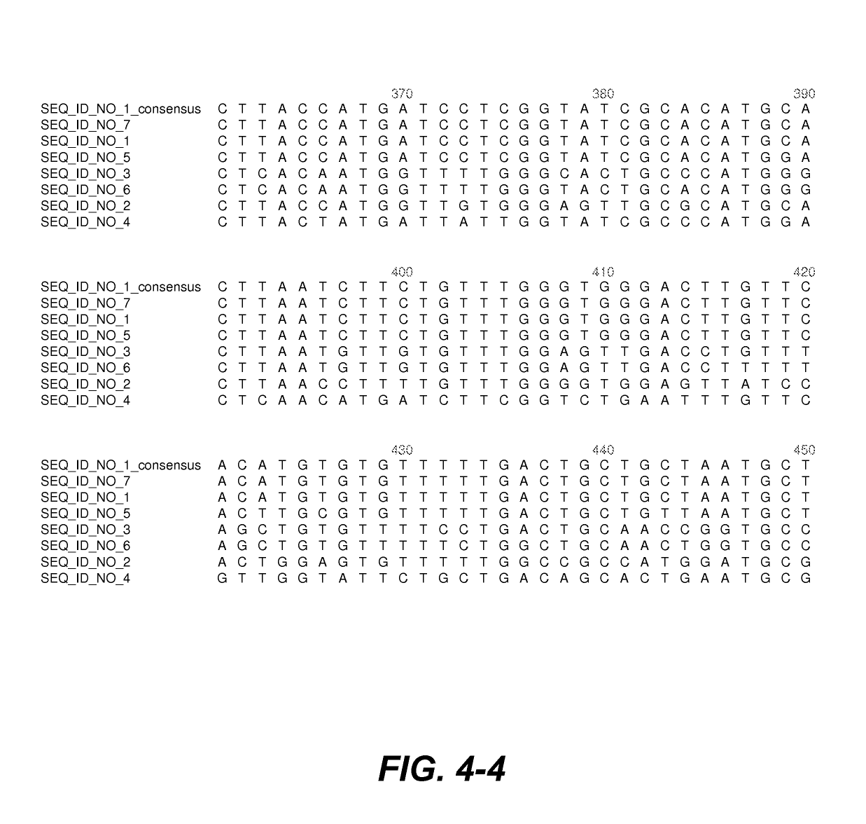

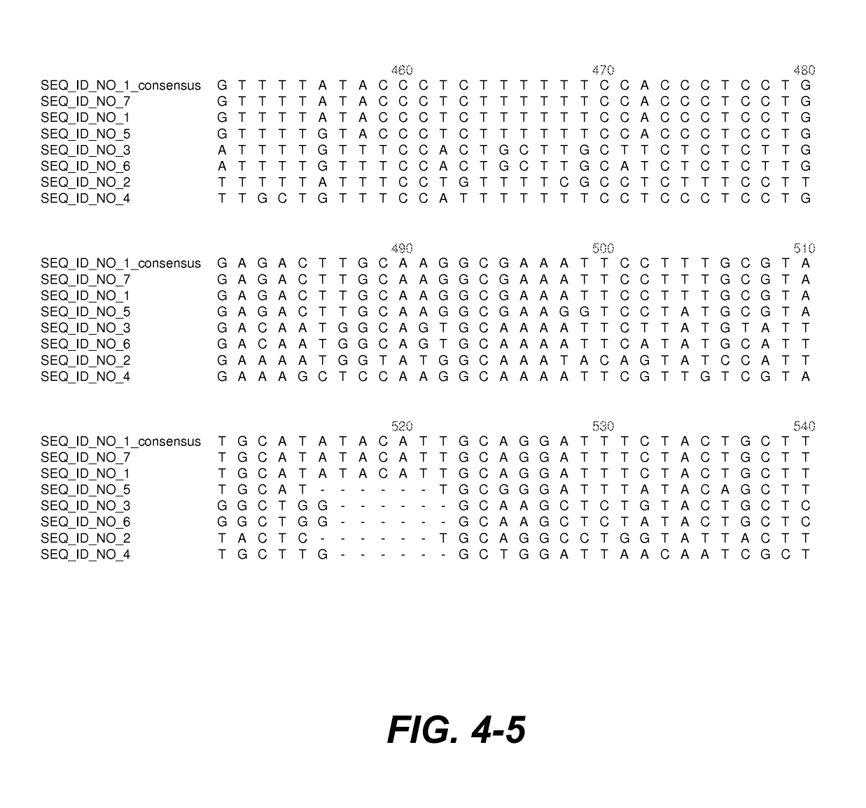

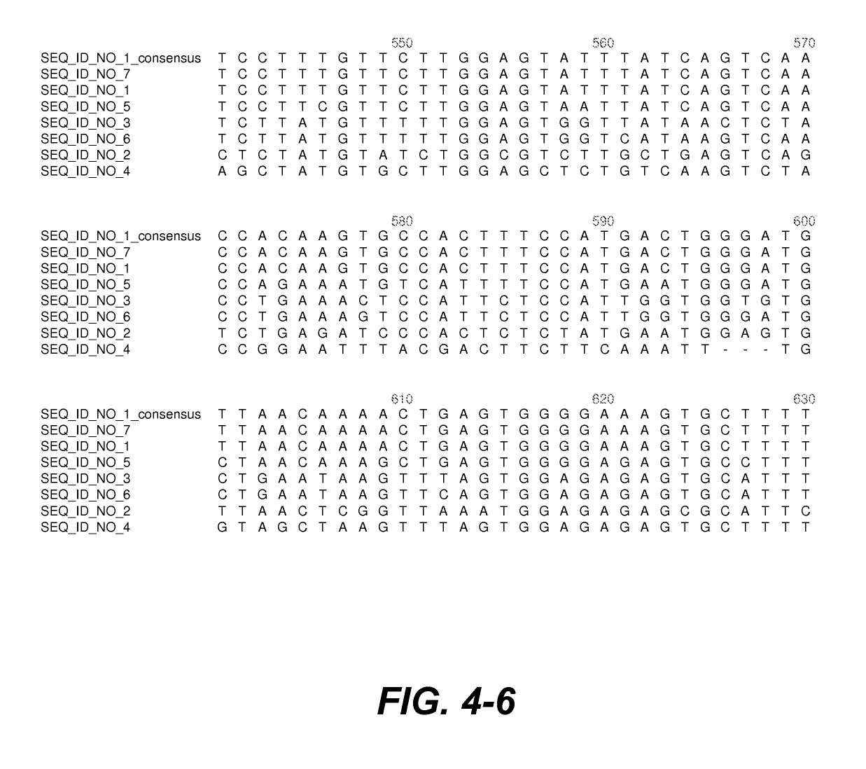

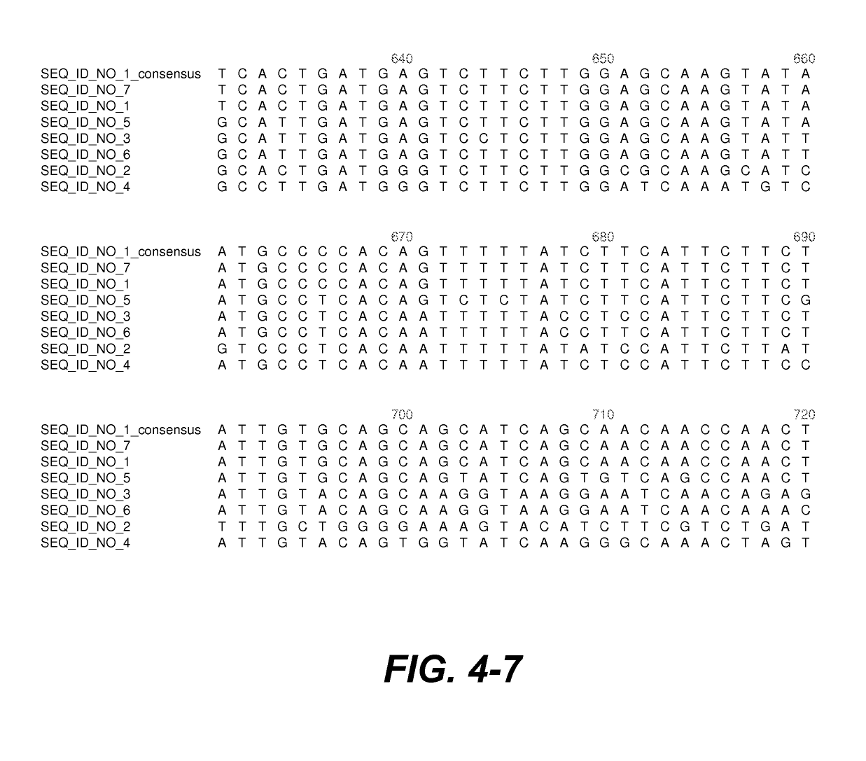

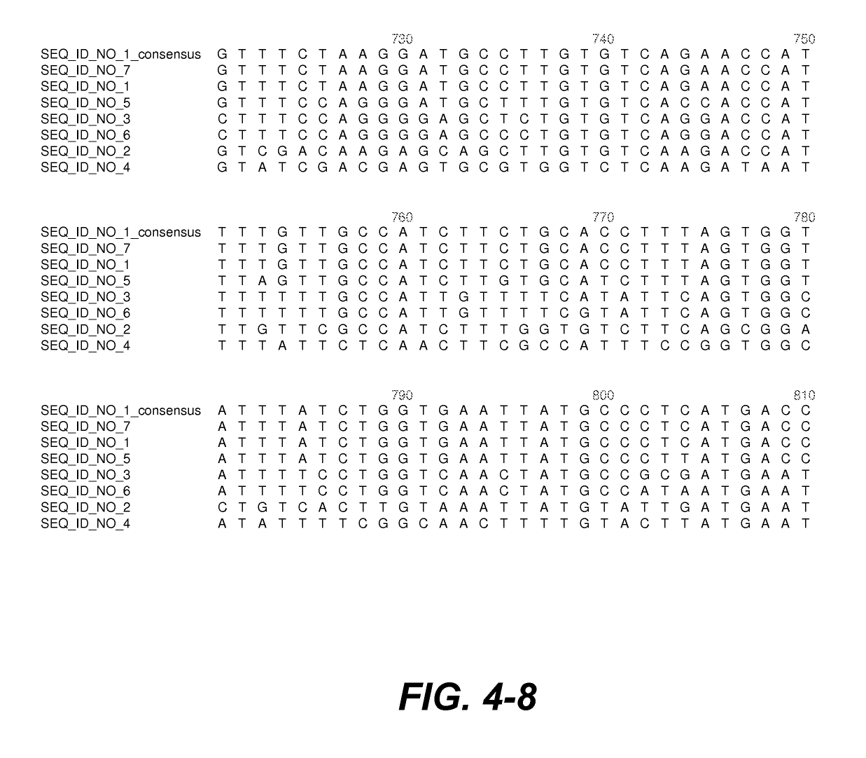

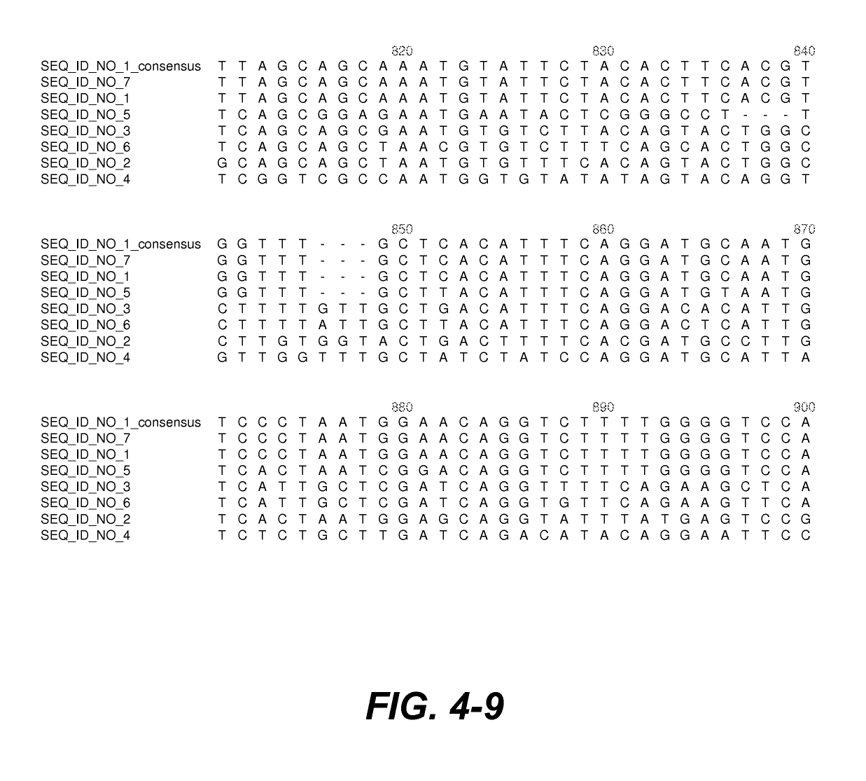

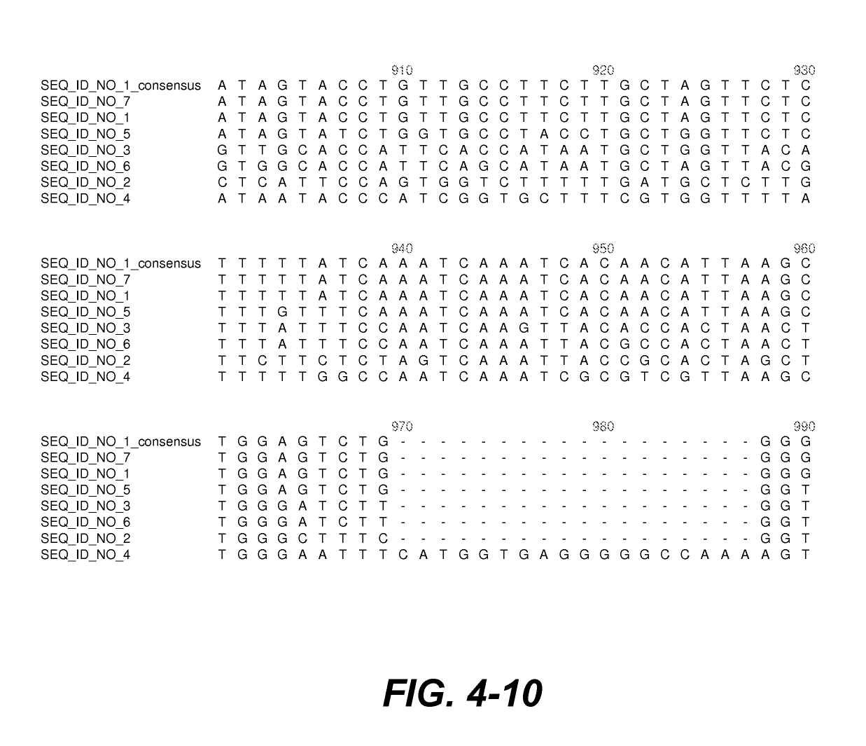

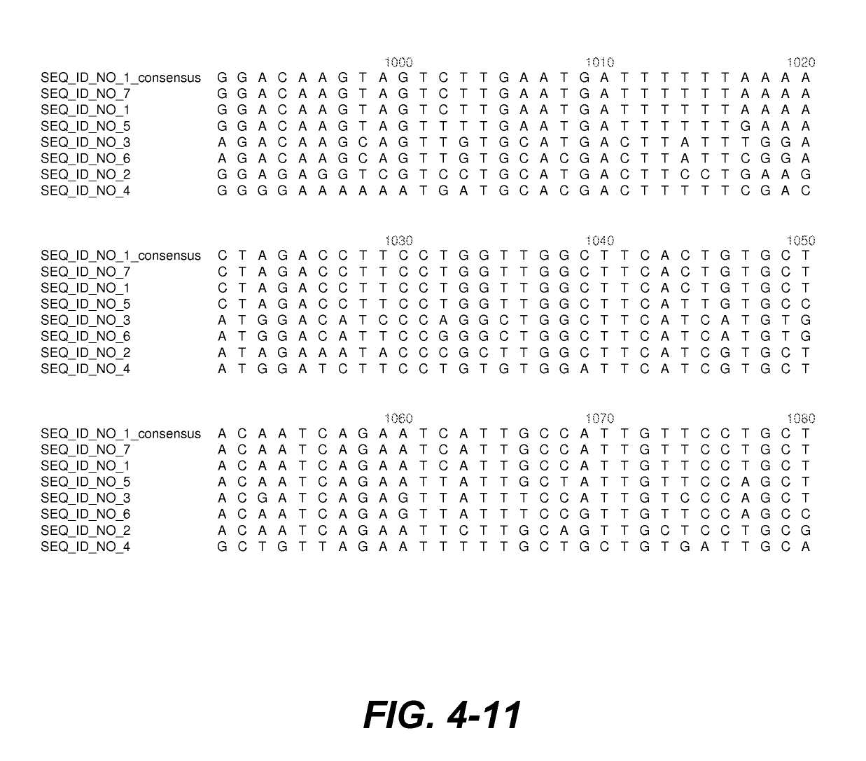

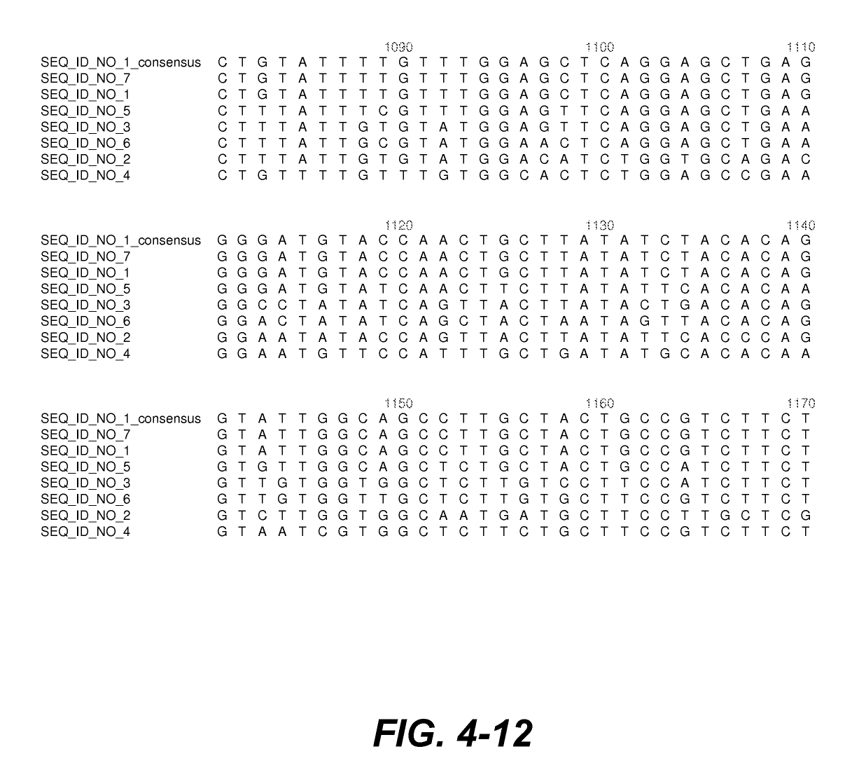

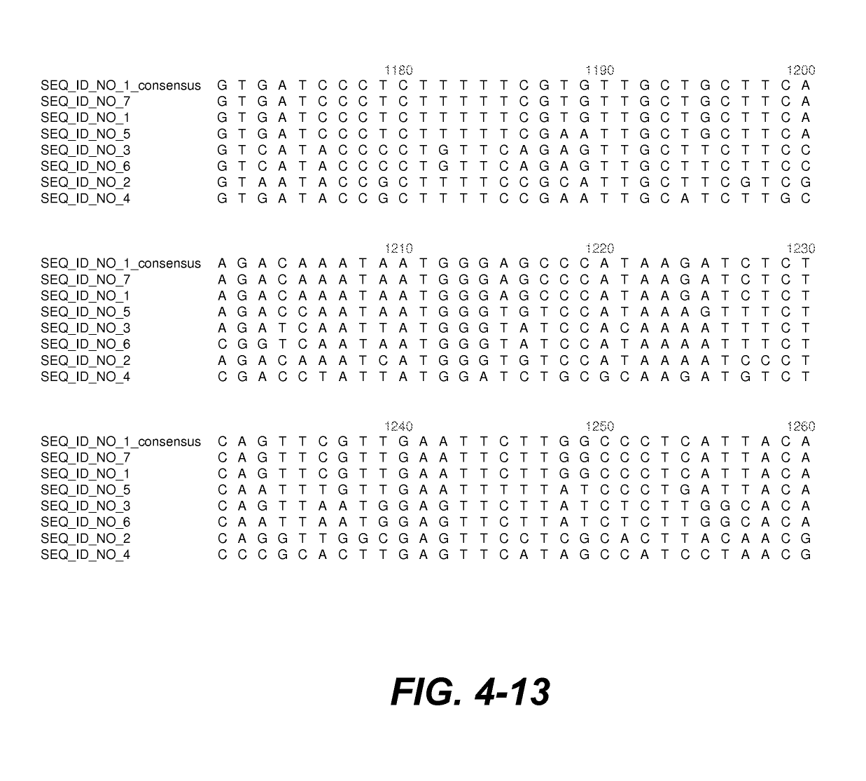

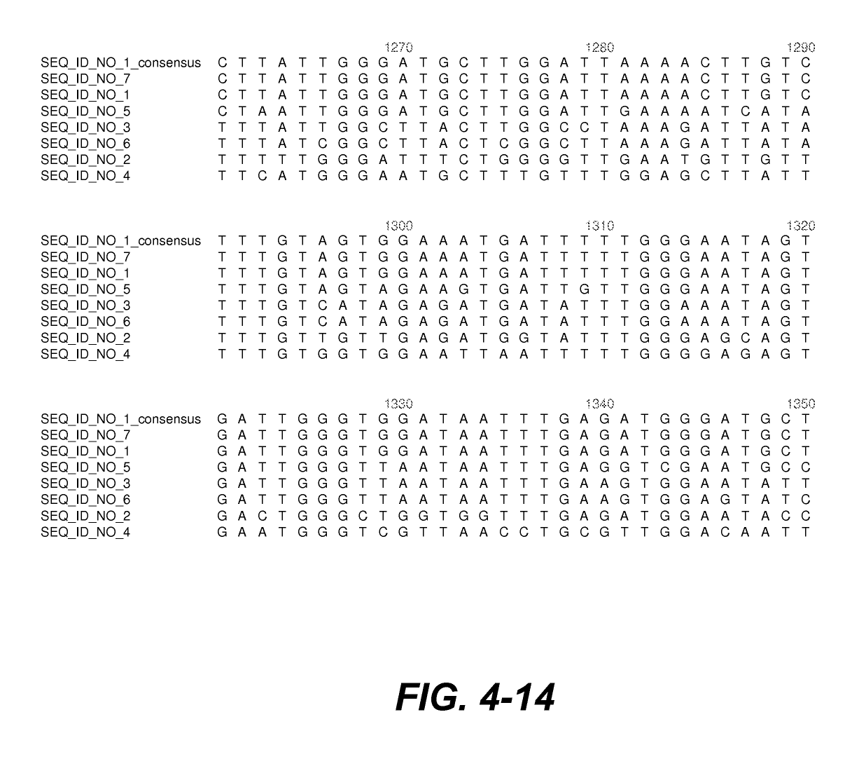

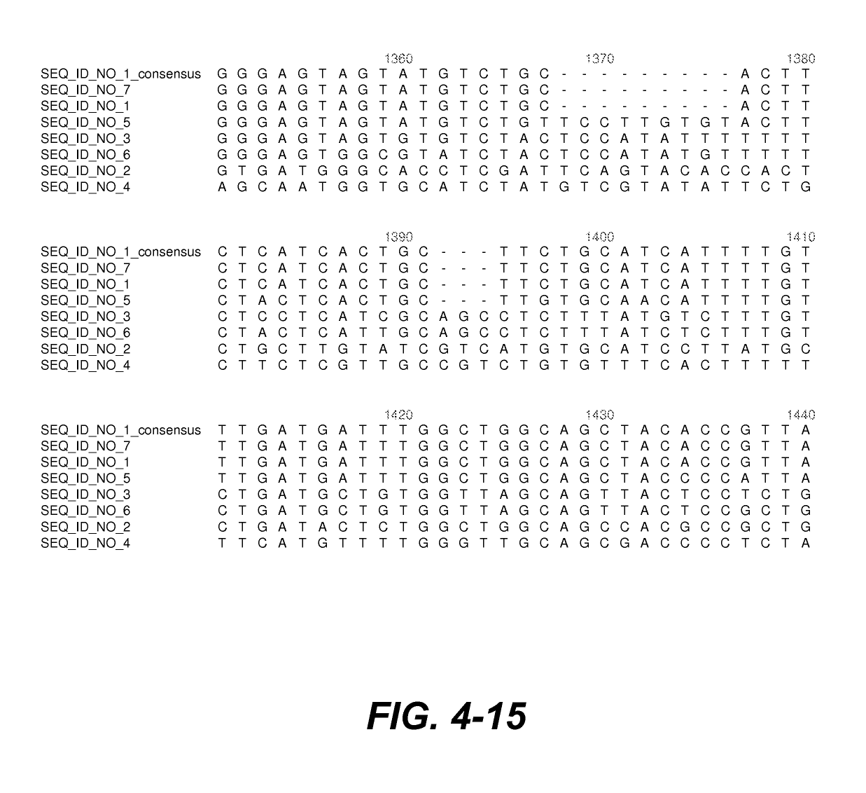

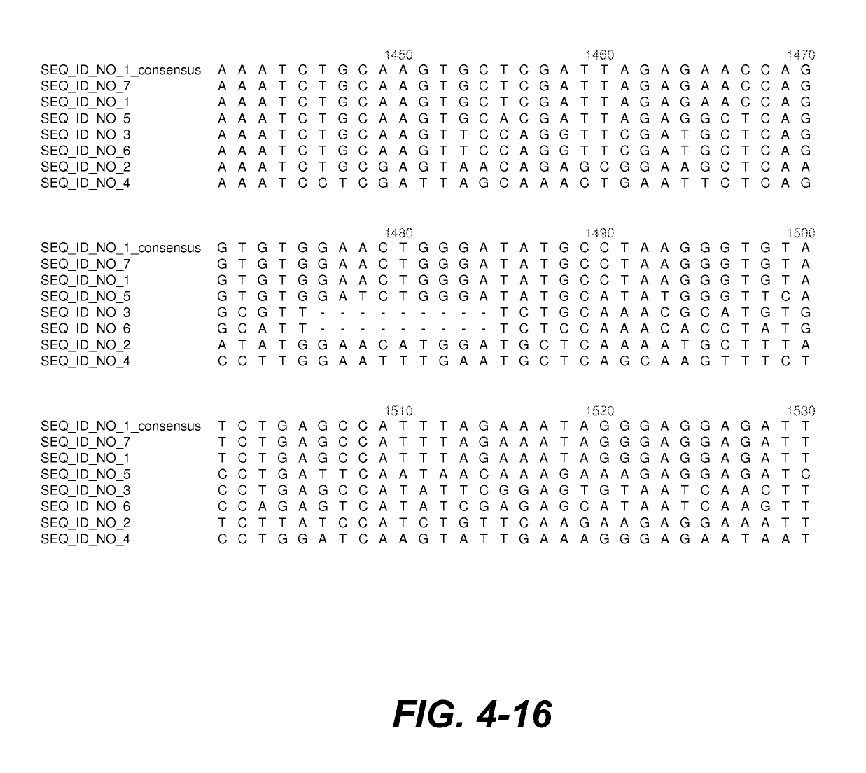

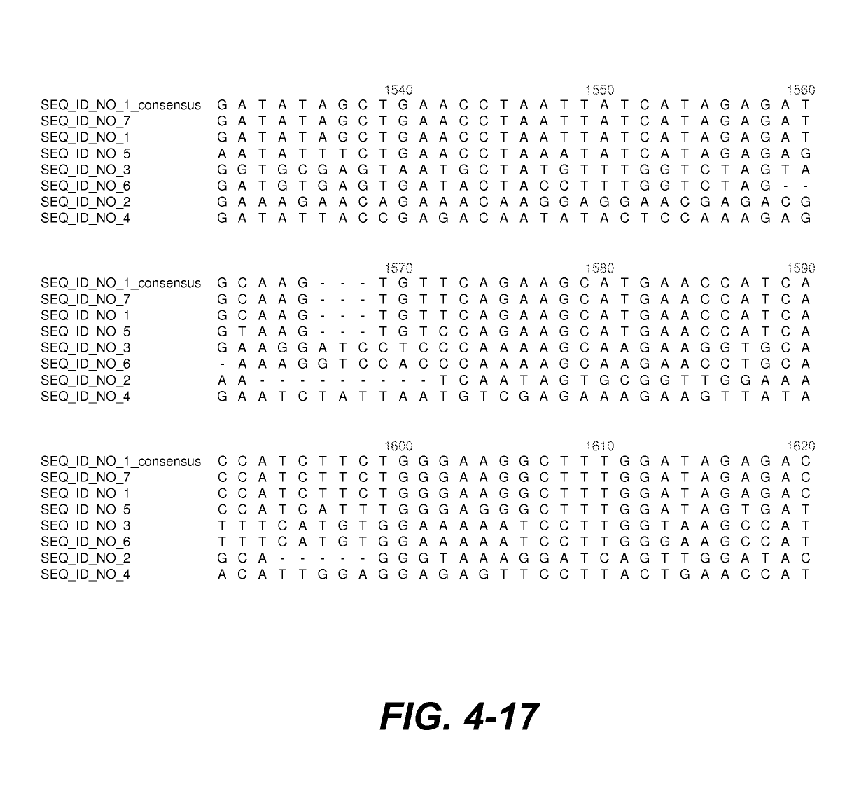

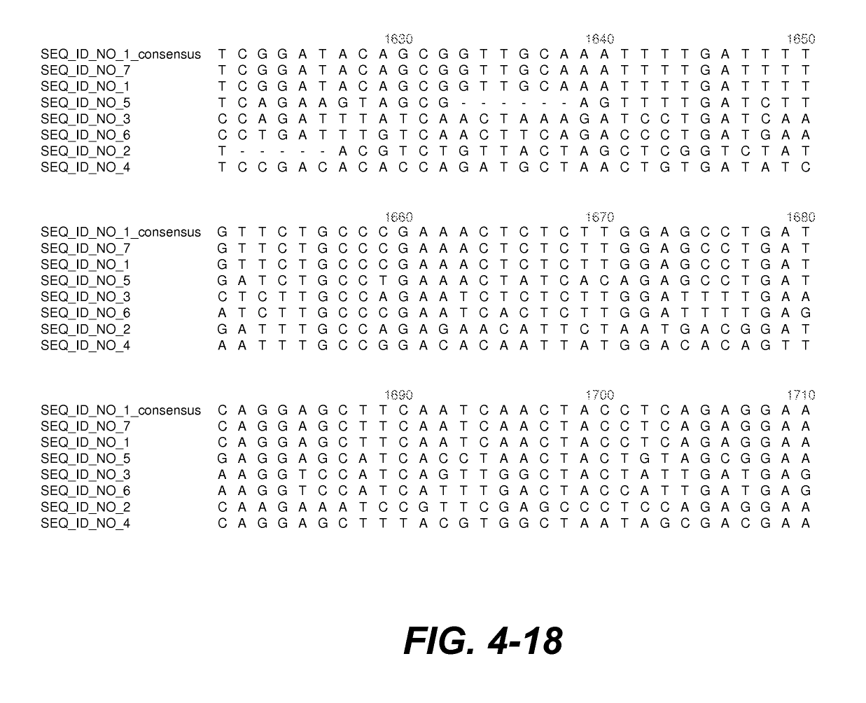

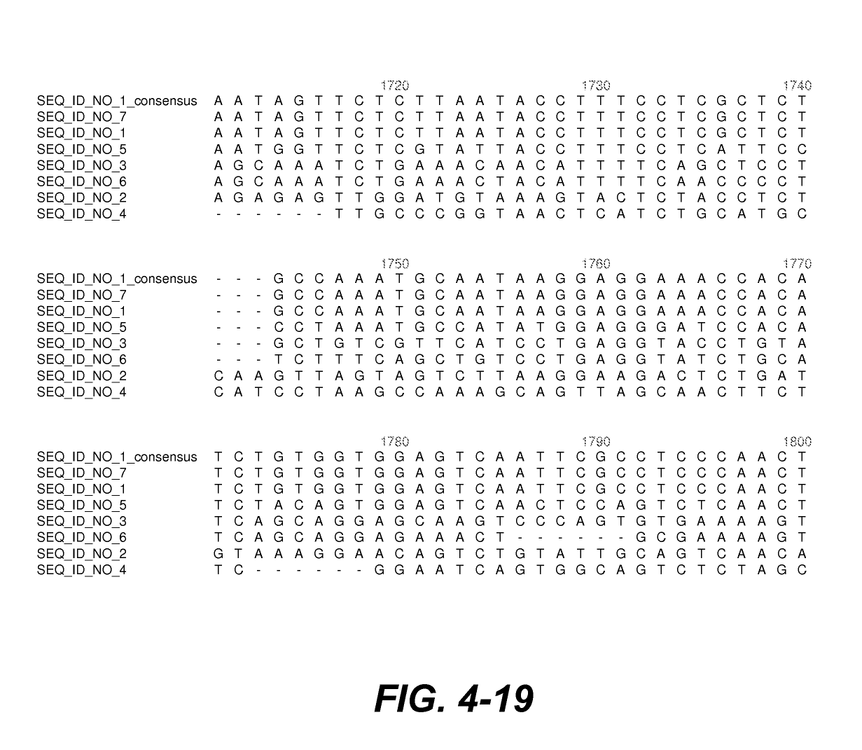

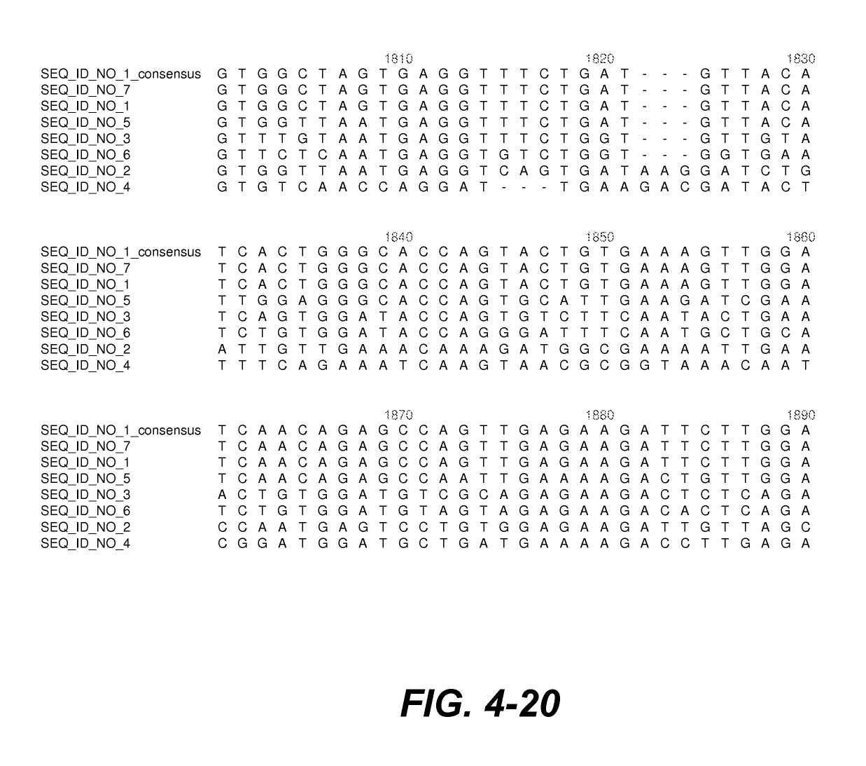

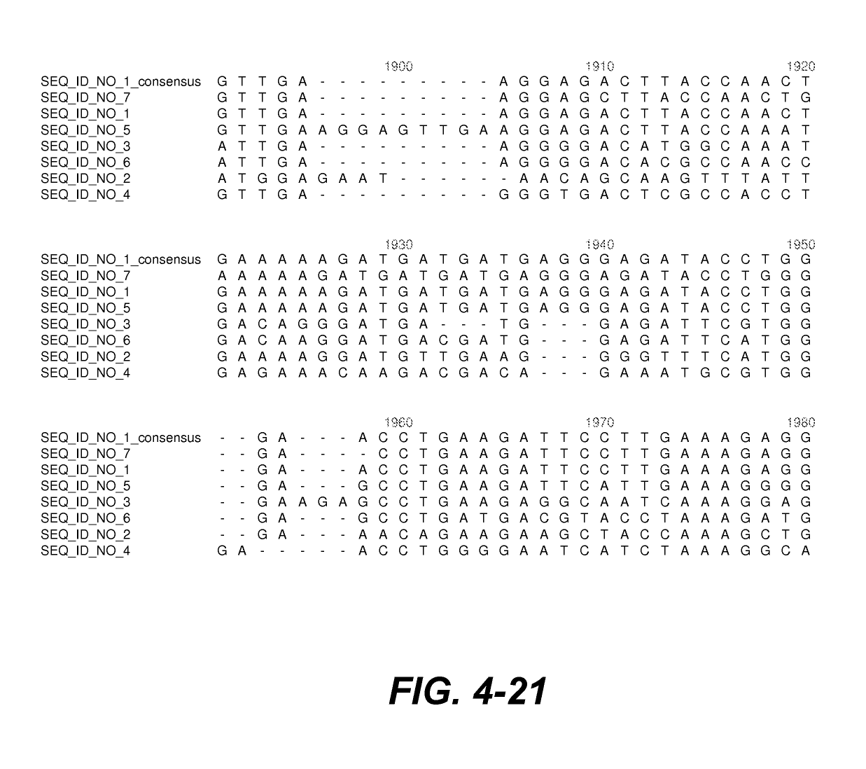

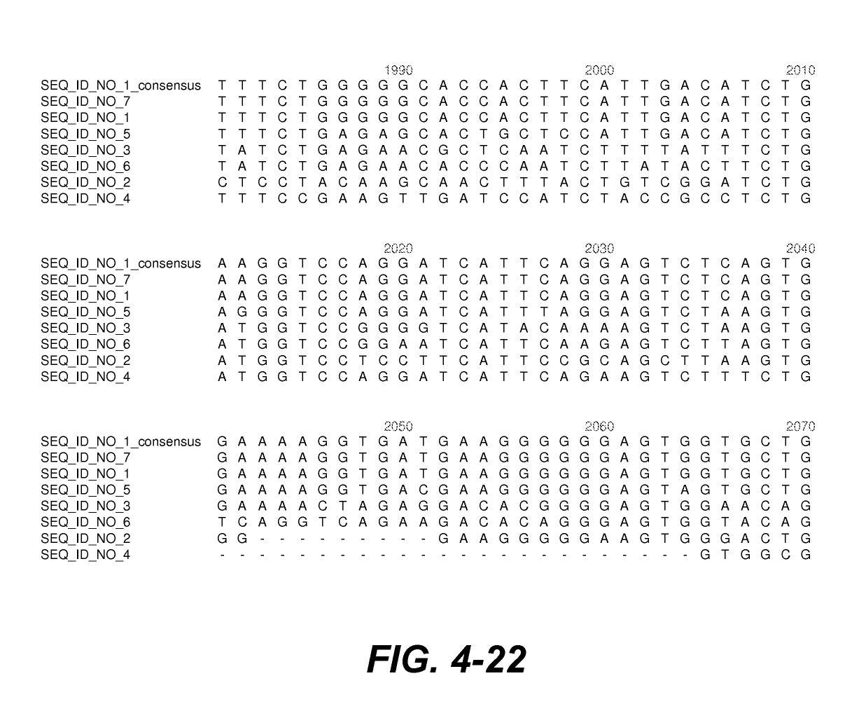

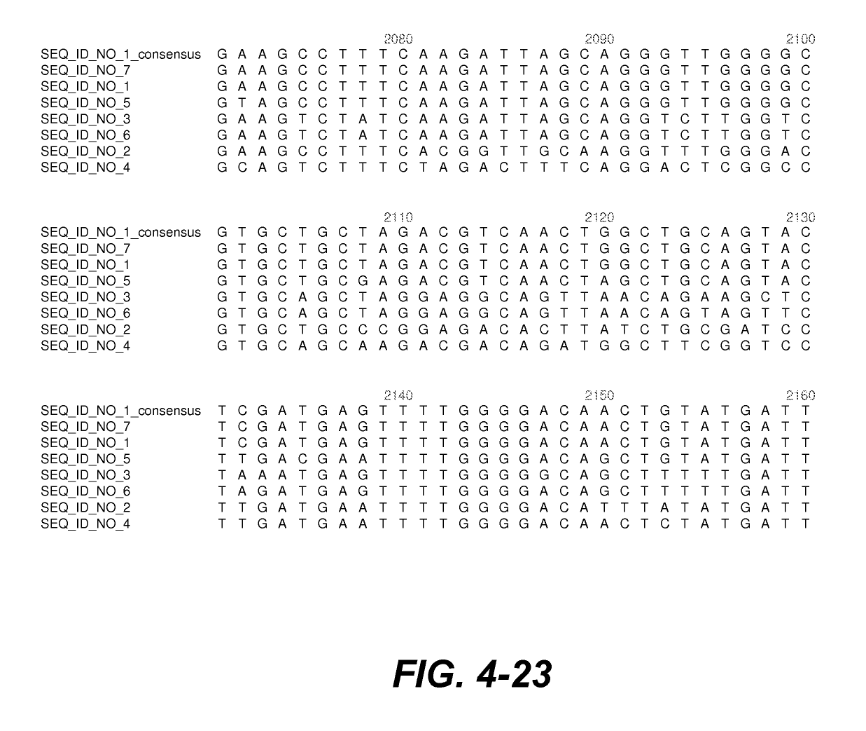

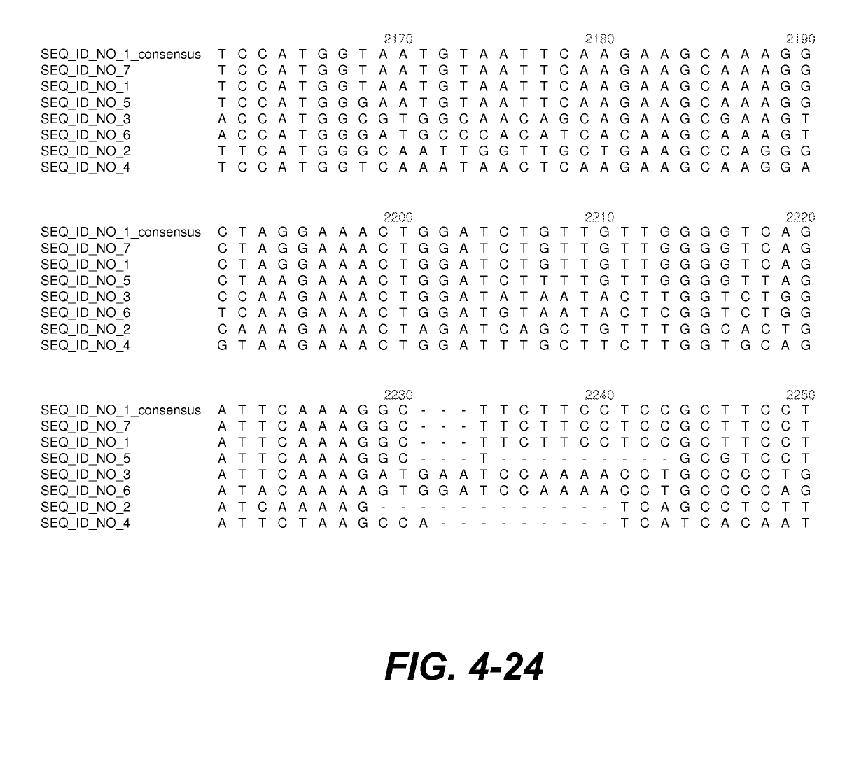

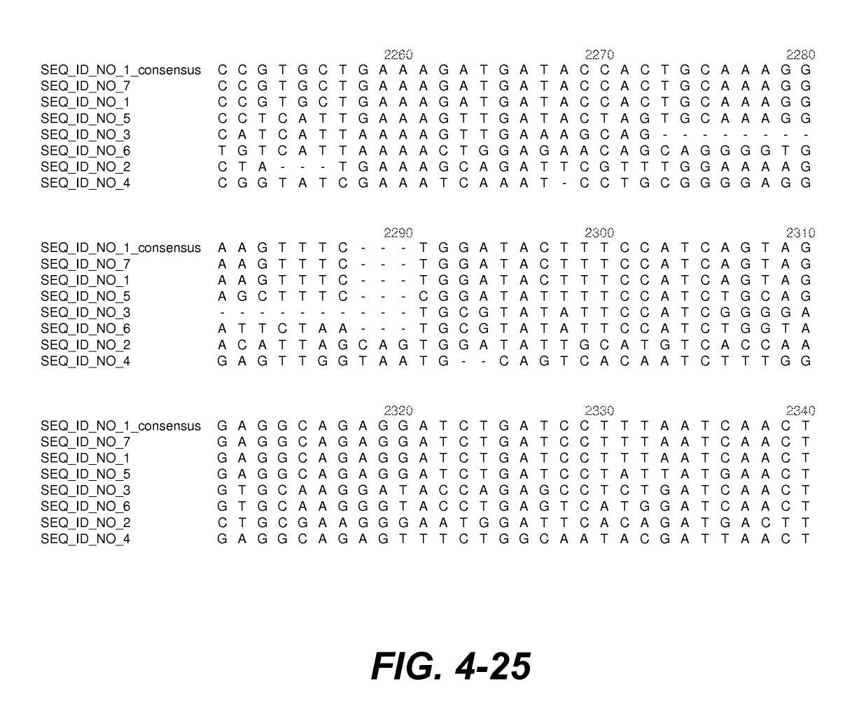

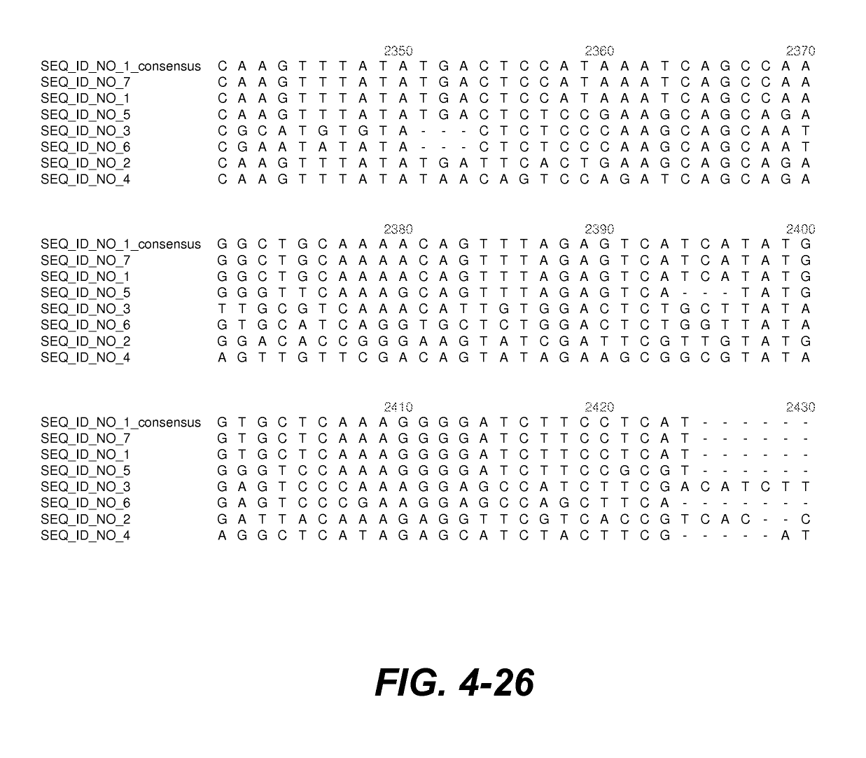

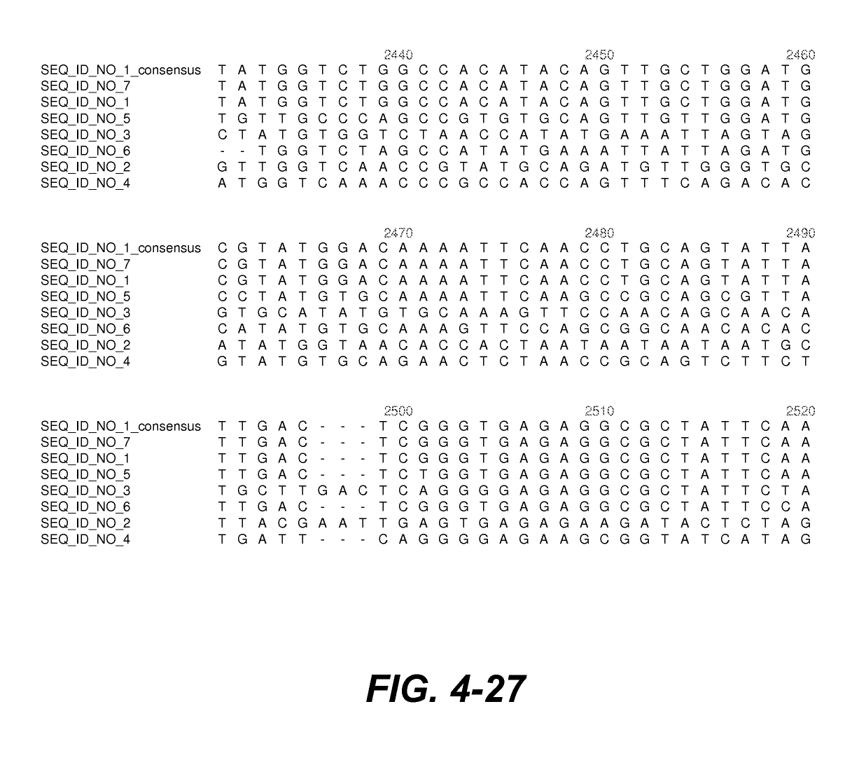

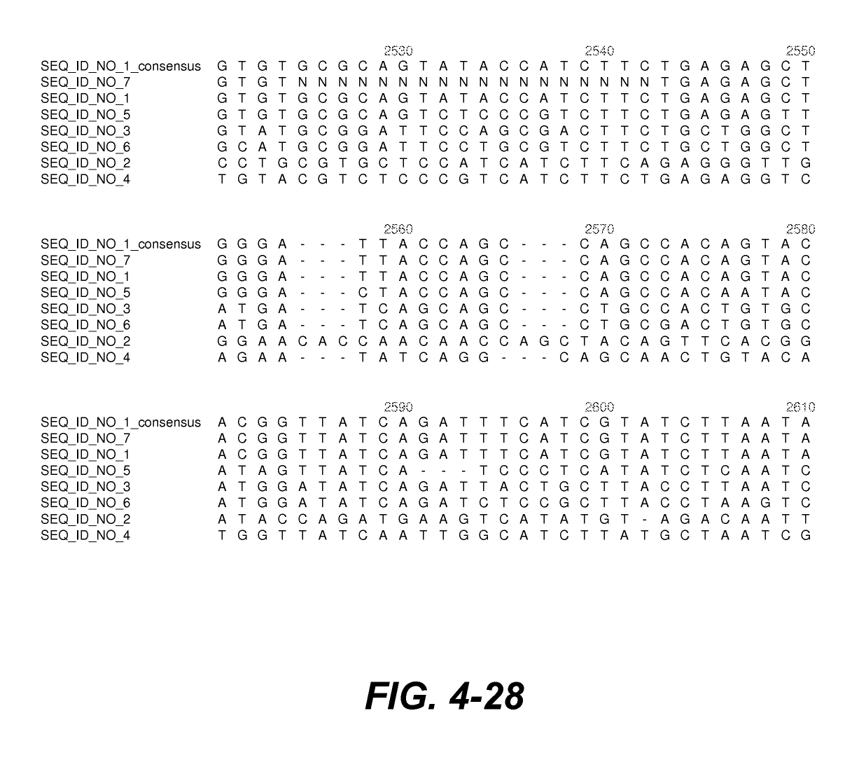

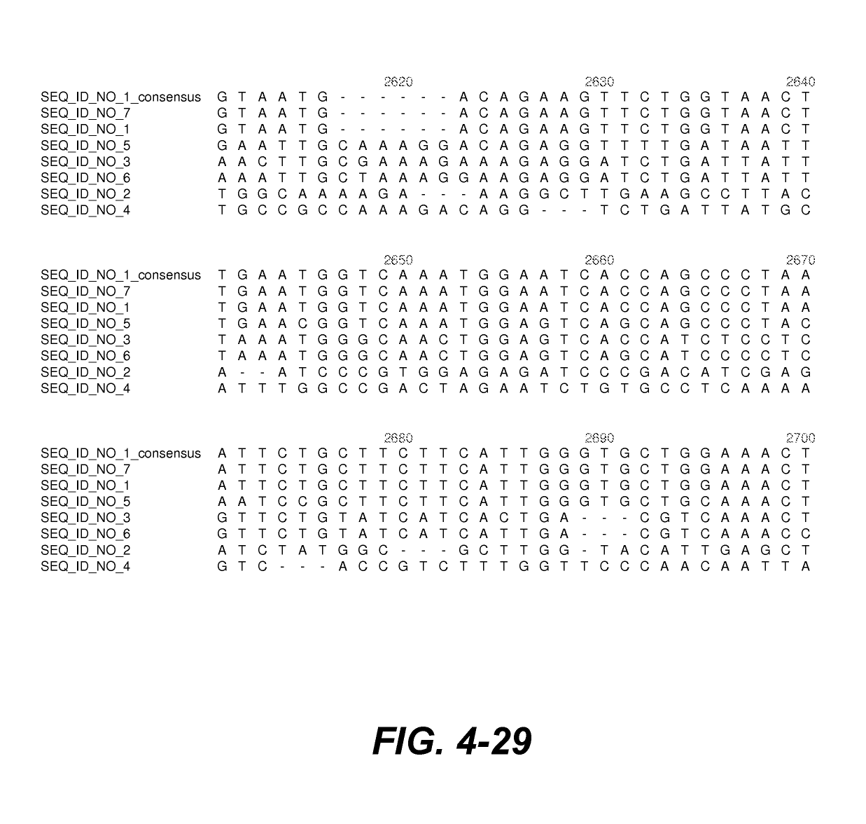

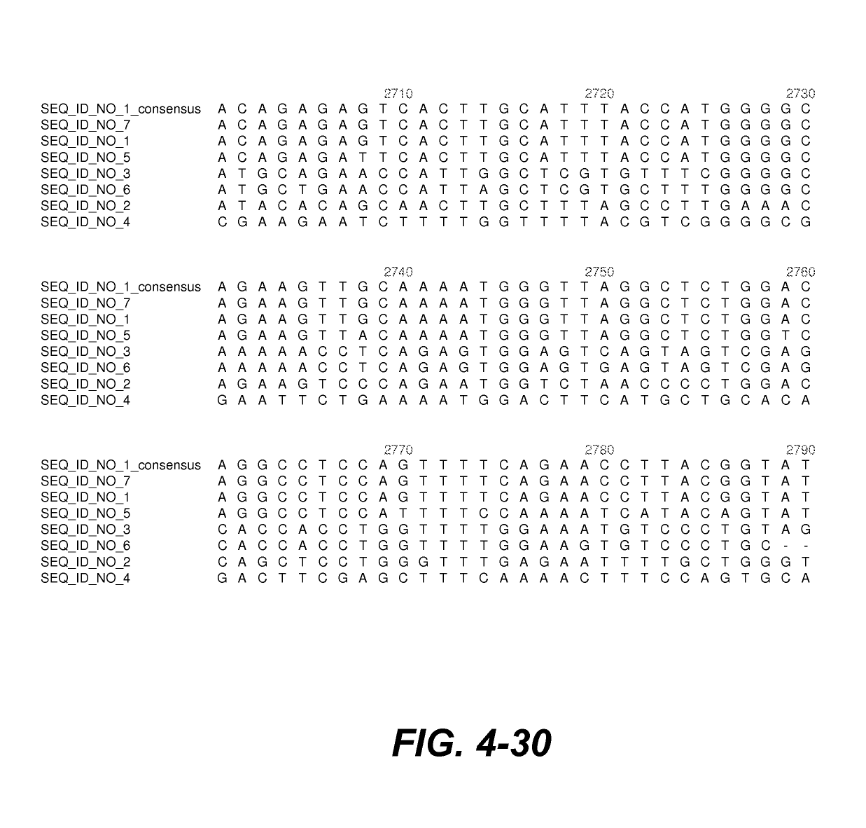

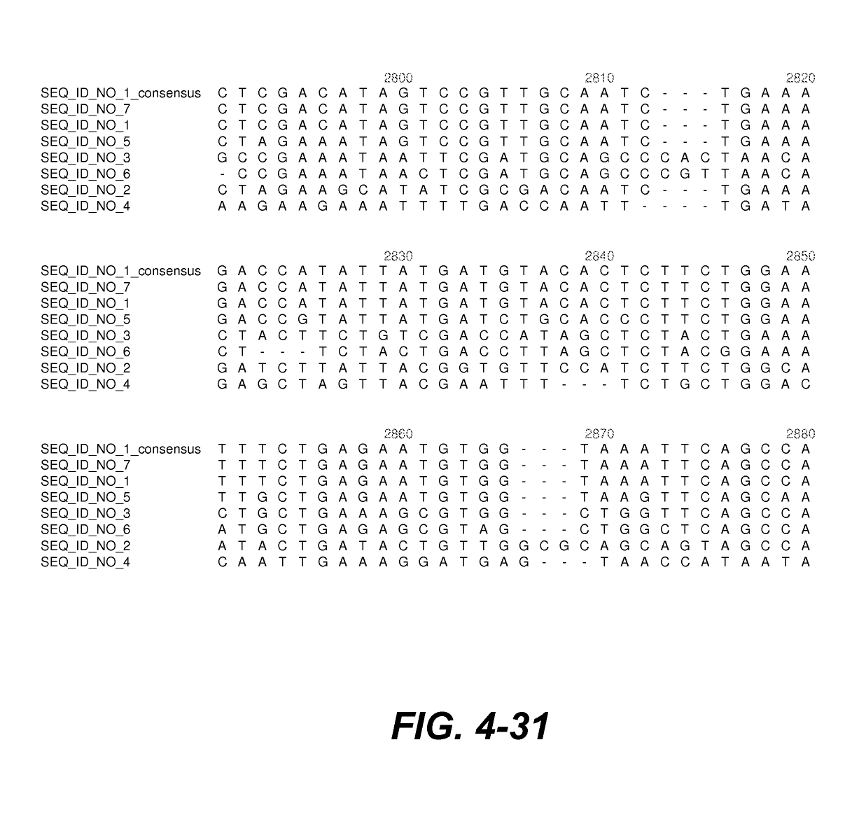

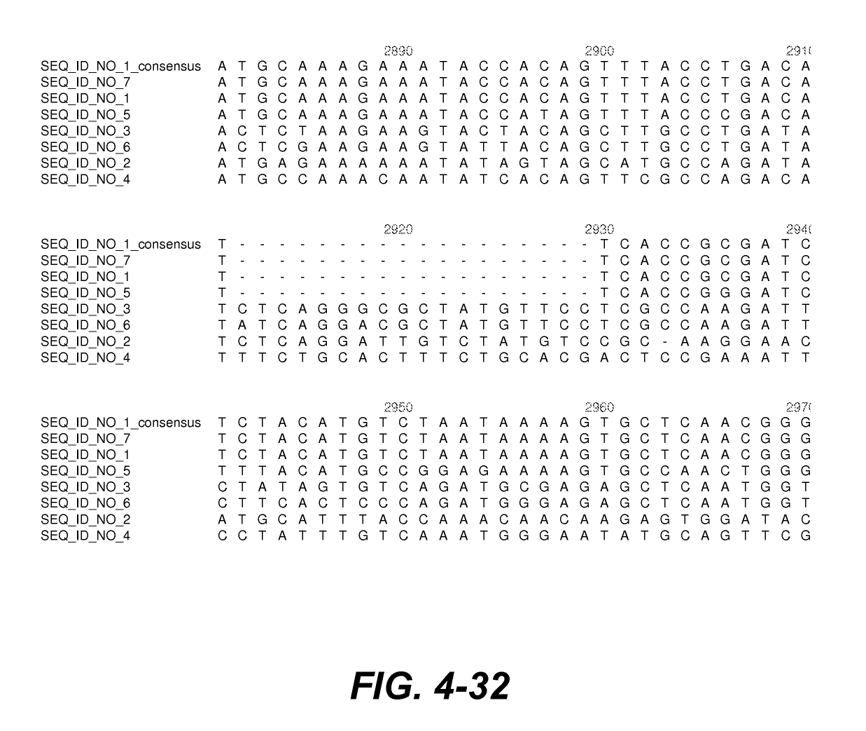

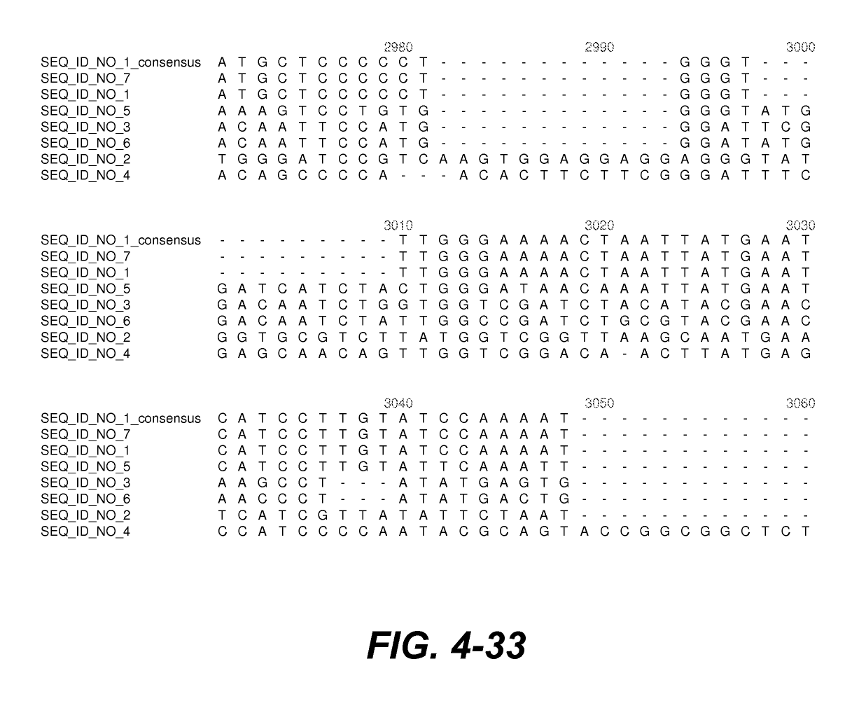

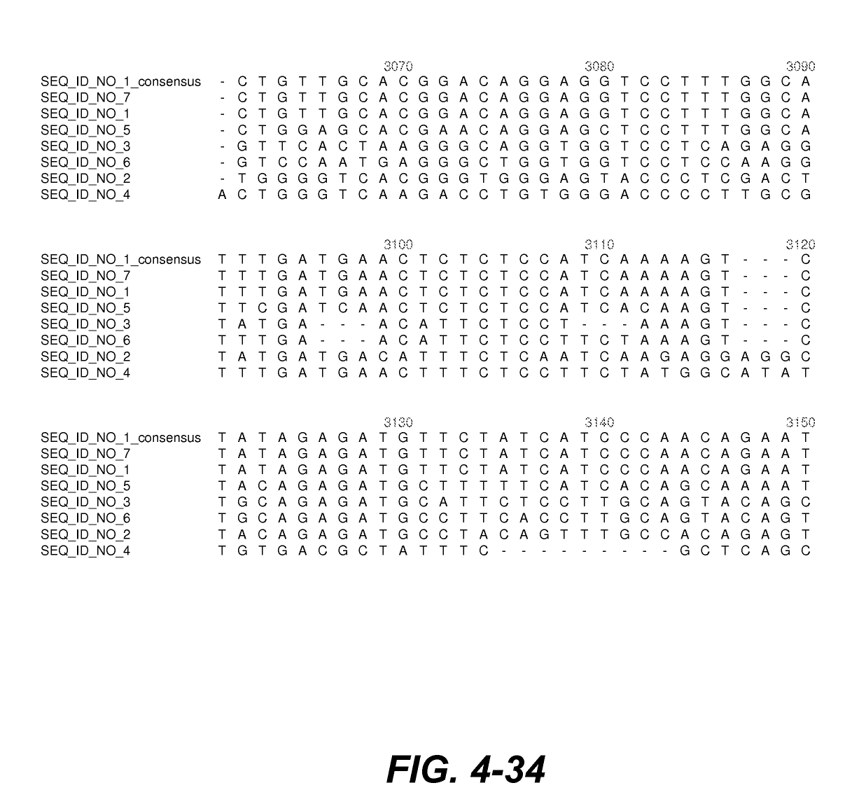

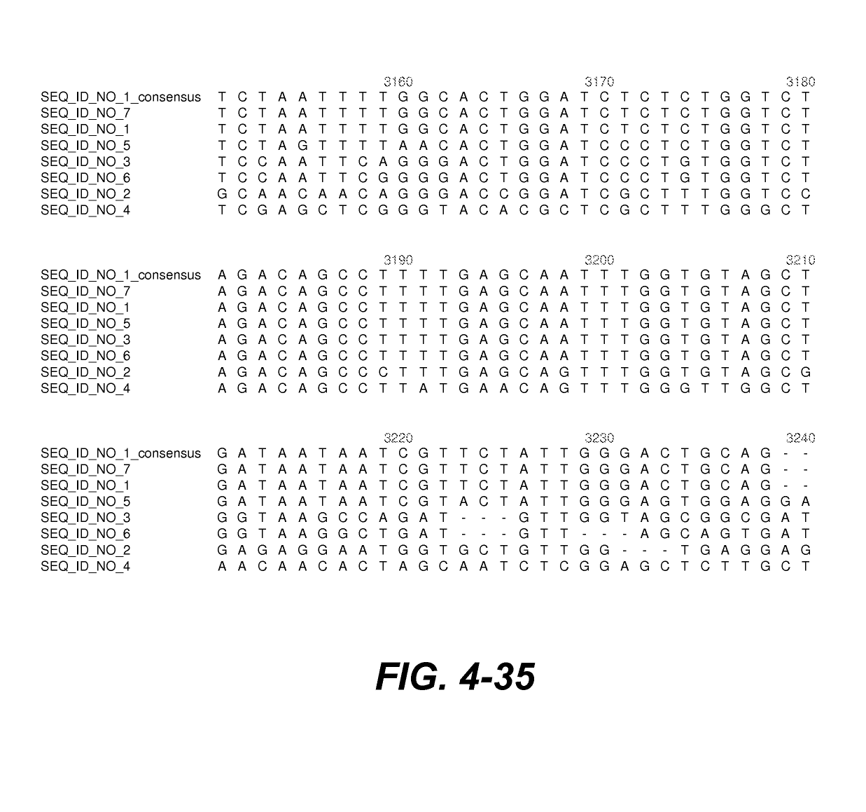

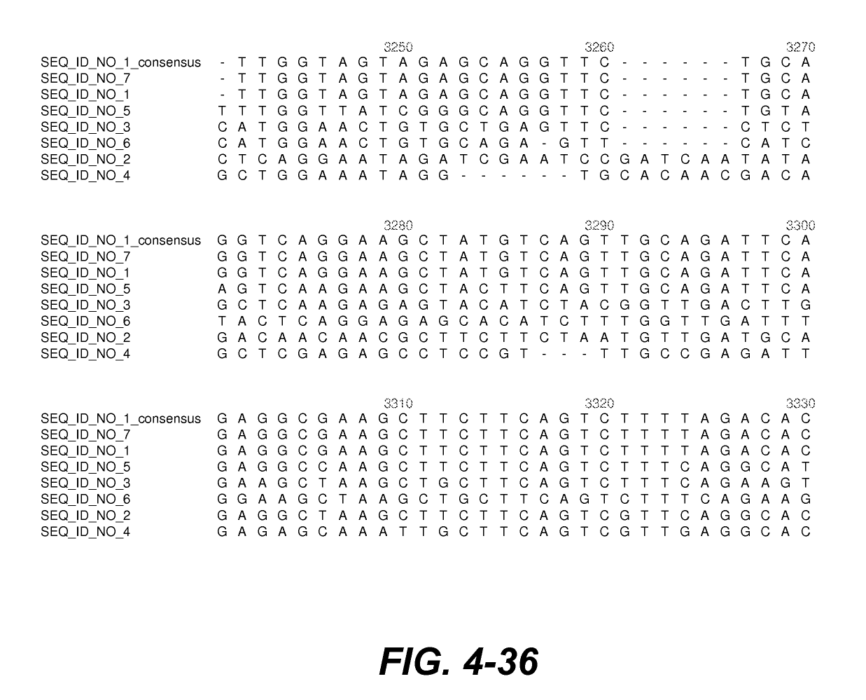

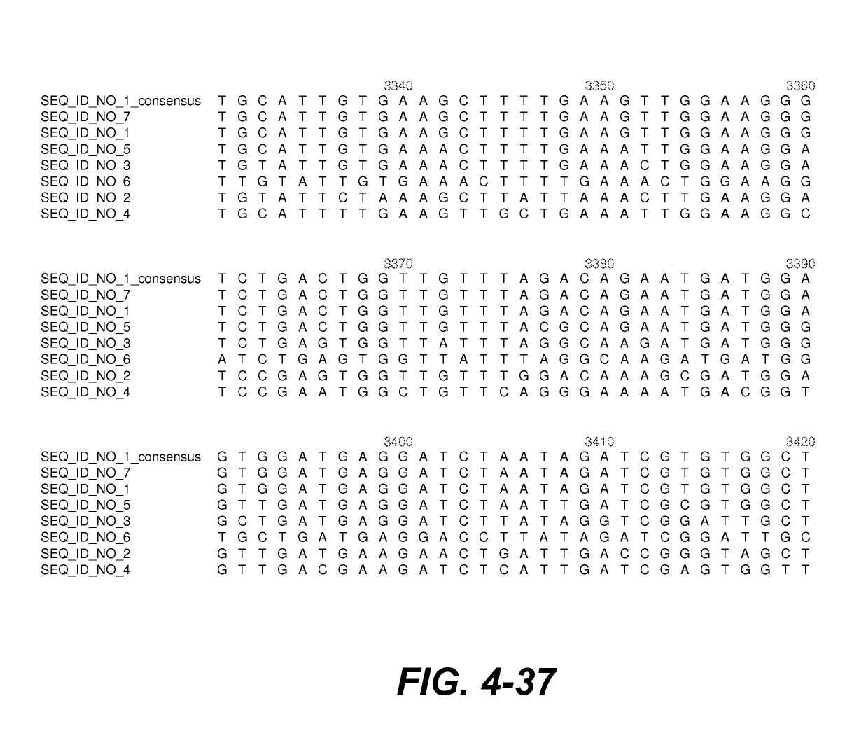

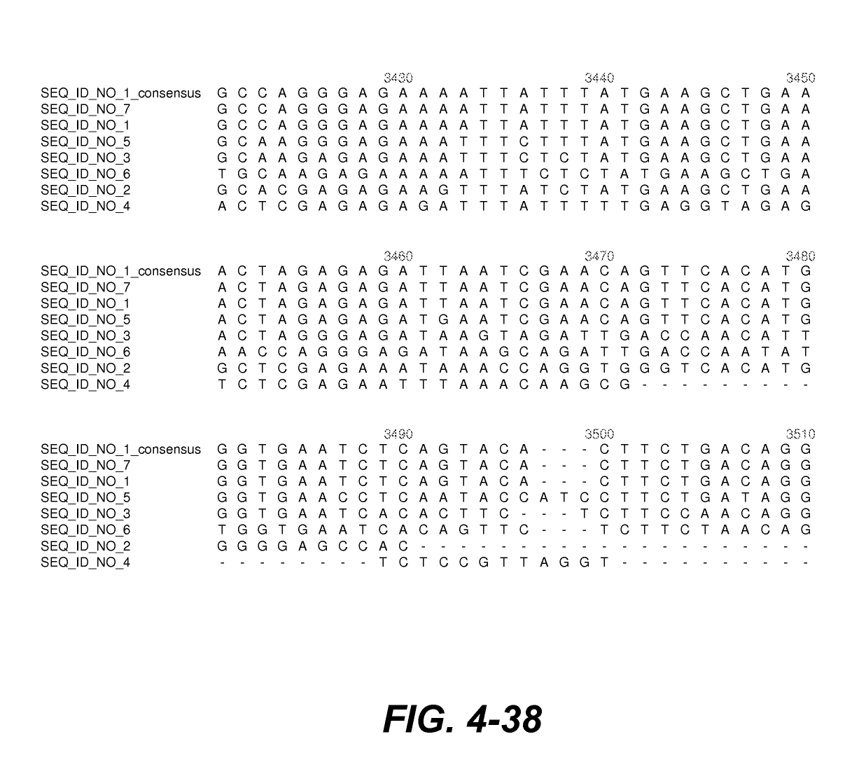

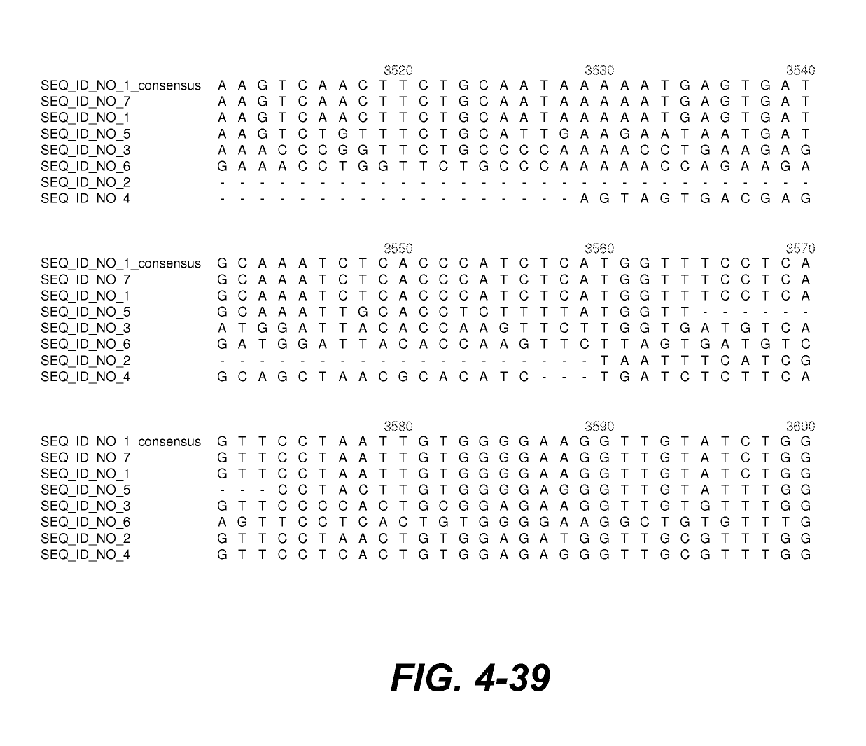

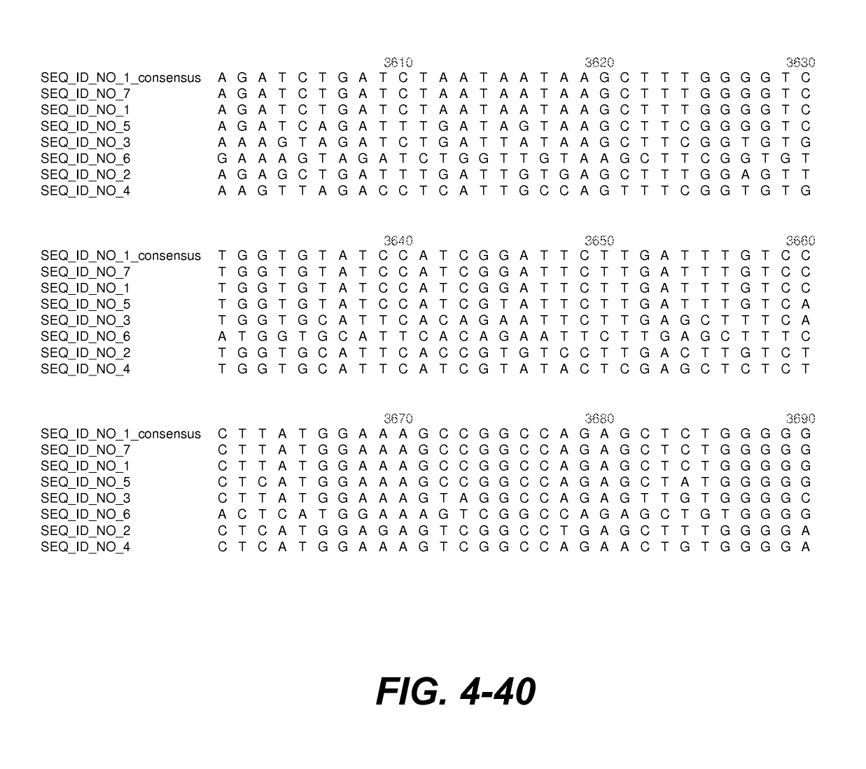

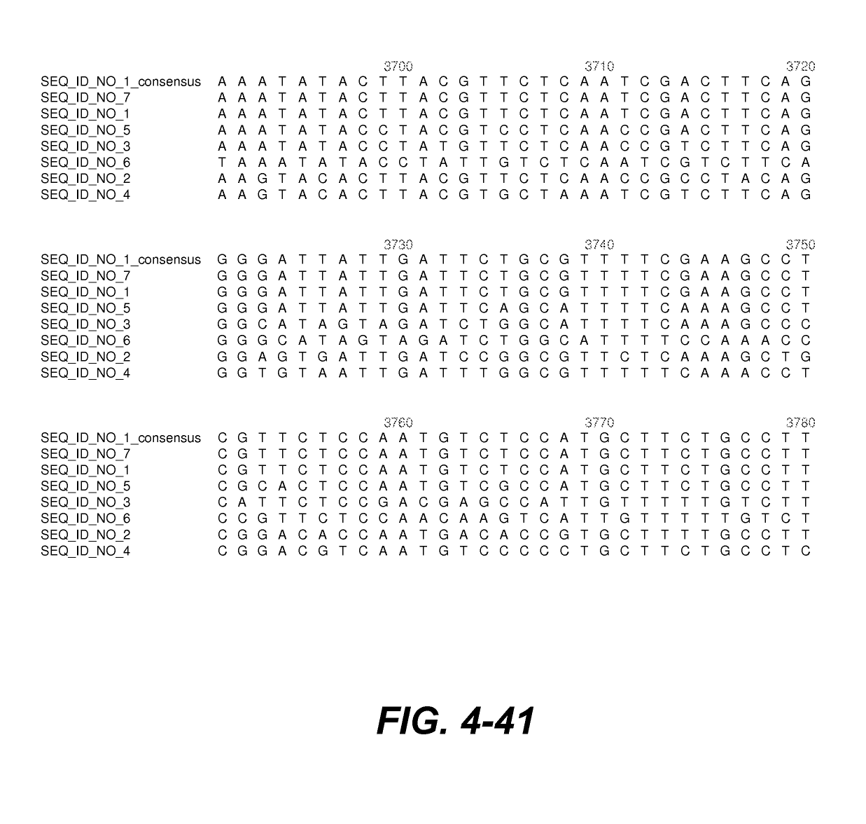

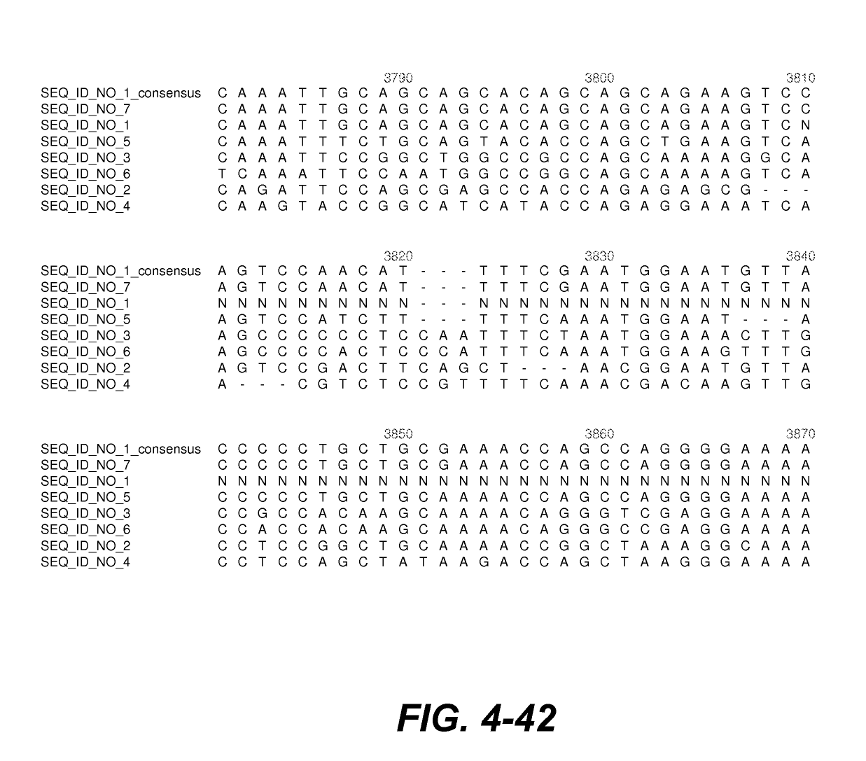

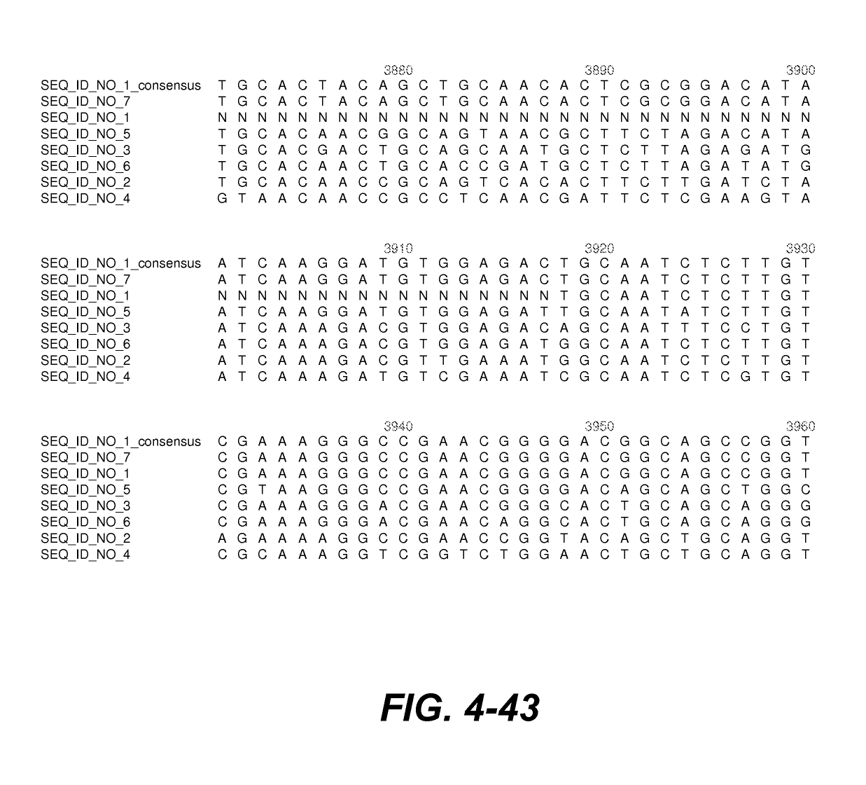

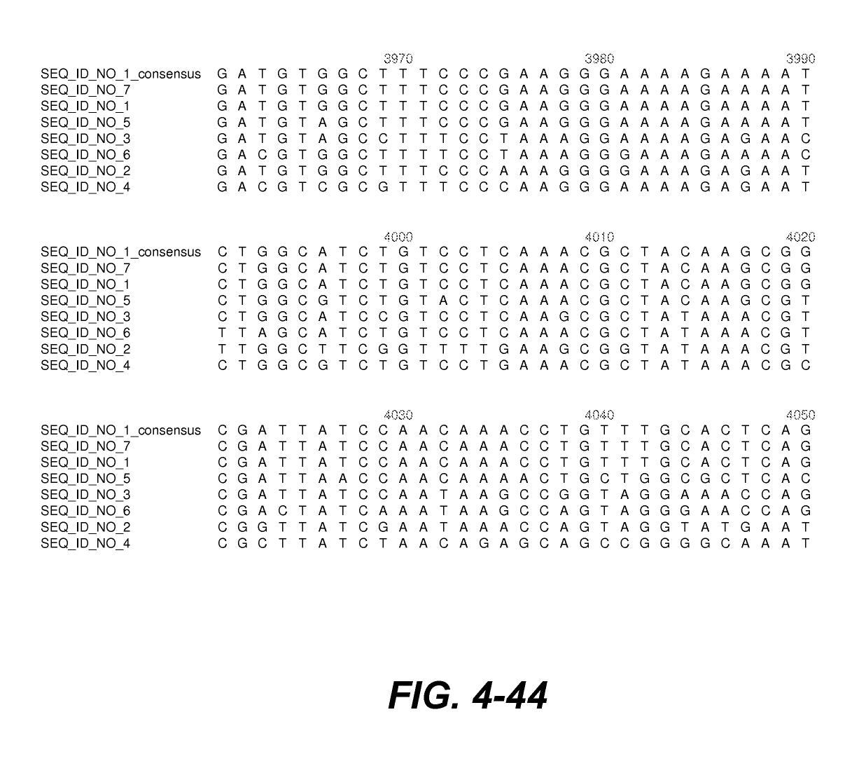

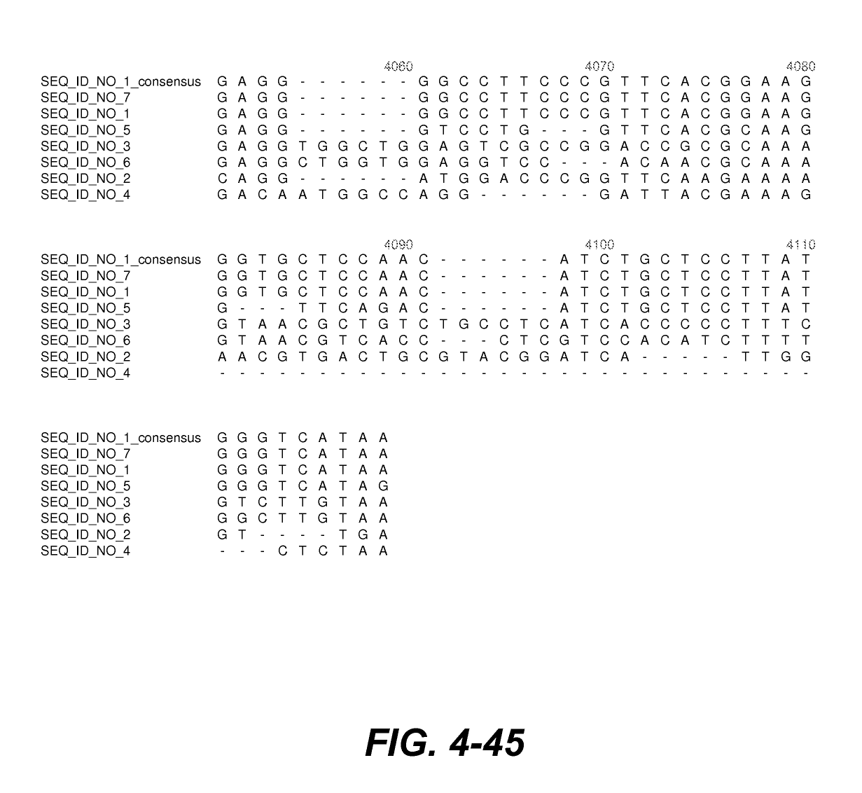

Polynucleotide trigger molecules can be identified by “tiling” gene targets in random length fragments, e.g. 200-300 polynucleotides in length, with partially overlapping regions, e.g. 25 or so nucleotide overlapping regions along the length of the target gene. Multiple gene target sequences can be aligned and polynucleotide sequence regions with homology in common are identified as potential trigger molecules for multiple targets. See, e.g. FIG. 4. Multiple target sequences can be aligned and sequence regions with poor homology are identified as potential trigger molecules for selectively distinguishing targets. To selectively suppress a single gene, trigger sequences may be chosen from regions that are unique to the target gene either from the transcribed region or the non-coding regions, e.g., promoter regions, 3′ untranslated regions, introns and the like.

Polynucleotides fragments are designed along the length of the full length coding and untranslated regions of a gene or family member as contiguous overlapping fragments of 200-300 polynucleotides in length or fragment lengths representing a percentage of the target gene. These fragments are applied in the vase solution or applied topically (as sense or anti-sense ssDNA or ssRNA, dsRNA, or dsDNA) to determine the relative effectiveness in providing delayed senescence and/or improved appearance. Fragments providing the desired activity may be further subdivided into 50-60 polynucleotide fragments which are evaluated for providing delayed senescence and/or improved appearance. The 50-60 base fragments with the desired activity may then be further subdivided into 19-30 base fragments which are evaluated for providing delayed senescence and/or improved appearance. Once relative effectiveness is determined, the fragments are utilized singly, or in combination in one or more pools to determine effective trigger composition or mixture of trigger polynucleotides for providing delayed senescence and/or improved appearance.

Triggers are developed to simultaneously suppress multiple gene family members by alignment of coding and/or non-coding sequences of gene families in the plant of interest, and choosing 200-300 base fragments from the most similar regions of the aligned sequences for evaluation by applying in the vase solution or applying topically (as sense or anti-sense ssDNA or ssRNA, dsRNA, or dsDNA) to determine their relative effectiveness in providing delayed senescence and/or improved appearance. The effective segments are subdivided into 50-60 base fragments, prioritized by greatest similarity, and re-evaluated in a topical application method. The effective 50-60 base fragments are subdivided into 19-30 base fragments, prioritized by greatest similarity, and again evaluated for providing delayed senescence and/or improved appearance. Once relative effectiveness is determined, the fragments may be utilized singly, or in combination with one or more other fragments to determine the trigger formulation for providing the trait phenotype.

Also, provided herein are methods for identifying a polynucleotide for providing delayed senescence and/or improved appearance in a cut flower. Populations of candidate polynucleotides that are essentially identical or essentially complementary to an EIN2 gene or transcript of the EIN2 gene can be generated by a variety of approaches, including but not limited to, any of the tiling, least homology, or most homology approaches provided herein. Such populations of polynucleotides can also be generated or obtained from any of the polynucleotides or genes provided herewith in SEQ ID NOs: 1-7. Such populations of polynucleotides can also be generated or obtained from any genes that are orthologous to the genes provided herewith in SEQ ID NOs: 1-7. Such polynucleotides can be topically applied to a surface of a cut flower or to the water in a composition comprising at least one polynucleotide from said population and, optionally, a transfer agent to obtain treated plants. Treated flowers that exhibit suppression of the EIN2 gene and/or exhibit an improvement in delayed senescence and/or improved appearance are identified, thus identifying a preferred polynucleotide that improves delayed senescence and/or improved appearance in a cut flower. Suppression of the EIN2 gene can be determined by any assay for the levels and/or activity of a EIN2 gene product (i.e. transcript or protein). Suitable assays for transcripts include, but are not limited to, semi-quantitative or quantitative reverse transcriptase PCR® (qRT-PCR) assays. Suitable assays for proteins include, but are not limited to, semi-quantitative or quantitaive immunoassays, biochemical activity assays, or biological activity assays. In certain embodiments, the polynucleotides can be applied alone. In other embodiments, the polynucleotides can be applied in pools of multiple polynucleotides. When a pool of polynucleotides provides for suppression of the EIN2 gene and/or an improvement in delayed senescence and/or improved appearance are identified, the pool can be de-replicated and retested as necessary or desired to identify one or more preferred polynucleotide(s) that improve delayed senescence and/or improved appearance in a cut flower.

While there is no upper limit on the concentrations and dosages of polynucleotide molecules that can be useful in the methods and compositions provided herein, lower effective concentrations and dosages will generally be sought for efficiency. The concentrations can be adjusted in consideration of the volume of water in a container or the volume of spray or treatment applied to a cut flower or the plant from which it is harvested. In one embodiment, a useful treatment for cut flowers using 15-22 mer polynucleotide molecules is about 2 nanomole (nmol) of polynucleotide molecules per mL, for example, from about 0.05 to 2 nmol polynucleotides per mL. Other embodiments for cut flowers include useful ranges of about 0.05 to about 100 nmol, or about 0.1 to about 50 nmol, or about 1 nmol to about 25 nmol, or about 1 nmol to about 10 nmol, or about 0.5 nmol to about 5 nmol or about 0.25 nmol to about 3 nmol of polynucleotides per mL. In certain embodiments, about 40 to about 50 nmol of a ssDNA polynucleotide is applied. In certain embodiments, about 0.1 nmol to about 0.25 nmol, or about 0.25 nmol to about 0.5 nmol, or about 0.5 nmol to about 1 nmol, or about 1 nmol to about 2 nmol, or about 2 nmol to about 3 nmol, about 3 nmol to about 4 nmol, or about 4 nmol to about 5 nmol, about 5 nmol to about 6 nmol of a 22-50mer dsRNA is applied. In certain embodiments, a composition containing about 0.5 to about 2.0 mg/mL, or about 0.14 mg/mL of dsRNA or ssDNA is applied. In certain embodiments, a composition of about 0.5 to about 1.5 mg/mL of a long dsRNA polynucleotide (i.e. about 50 to about 200 or more nucleotides) is applied. In certain embodiments, about 1 nmol to about 5 nmol of a dsRNA is applied. In certain embodiments, the polynucleotide composition as applied to the cut flower contains the at least one polynucleotide at a concentration of about 0.01 to about 10 milligrams per milliliter, or about 0.05 to about 2 milligrams per milliliter, or about 0.1 to about 2 milligrams per milliliter. When using long dsRNA molecules that can be processed into multiple oligonucleotides, lower concentrations can be used. To illustrate embodiments of the invention, the factor 1×, when applied to oligonucleotide molecules is arbitrarily used to denote a treatment of 0.8 nmol of polynucleotide molecule per dozen cut flowers; 10×, 8 nmol of polynucleotide molecule per dozen cut flowers; and 100×, 80 nmol of polynucleotide molecule per dozen cut flowers.

In some embodiments, the polynucleotide compositions of this invention are useful in liquid compositions, such as liquids that comprise polynucleotide molecules, alone or in combination with one or more other components. In some embodiments, the polynucleotide compositions of this invention are useful in dry compositions, such as dry compositions that comprise polynucleotide molecules, alone or in combination with one or more other components. In certain embodiments of the methods, one component is a transfer agent.

As used herein, a transfer agent is an agent that, when combined with a polynucleotide in a composition that is topically applied to a target plant surface, facilitates entry of a polynucleotide to a plant cell. In certain embodiments, a transfer agent is an agent that conditions the surface of plant tissue, e. g., leaves, stems, or petals, to permeation by the polynucleotide molecules into plant cells. In some embodiments, the transfer of polynucleotides into plant cells can be facilitated by the prior or contemporaneous application of a polynucleotide-transferring agent to the plant tissue. In some embodiments the transferring agent is applied subsequent to the application of the polynucleotide composition. The polynucleotide transfer agent enables a pathway for polynucleotides through cuticle wax barriers, stomata and/or cell wall or membrane barriers into plant cells. However, as cells at the cut-end of a cut flower are exposed, a transfer agent may not be required to facilitate entry into a plant cell.

Suitable transfer agents to facilitate transfer of the polynucleotide into a plant cell include agents that increase permeability of the exterior of the plant or that increase permeability of plant cells to oligonucleotides or polynucleotides. Such agents to facilitate transfer of the composition into a plant cell include a chemical agent, or a physical agent, or combinations thereof. Chemical agents for conditioning or transfer include (a) surfactants, (b) an organic solvent or an aqueous solution or aqueous mixtures of organic solvents, (c) oxidizing agents, (d) acids, (e) bases, (f) oils, (g) enzymes, or combinations thereof. Embodiments of the method can optionally include an incubation step, a neutralization step (e.g., to neutralize an acid, base, or oxidizing agent, or to inactivate an enzyme), a rinsing step, or combinations thereof. Embodiments of agents or treatments for conditioning of a plant to permeation by polynucleotides include emulsions, reverse emulsions, liposomes, and other micellar-like compositions. Embodiments of agents or treatments for conditioning of a plant to permeation by polynucleotides include counter-ions or other molecules that are known to associate with nucleic acid molecules, e. g., inorganic ammonium ions, alkyl ammonium ions, lithium ions, polyamines such as spermine, spermidine, or putrescine, and other cations. Organic solvents useful in conditioning a plant to permeation by polynucleotides include DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions). Naturally derived or synthetic oils with or without surfactants or emulsifiers can be used, e. g., plant-sourced oils, crop oils (such as those listed in the 9^{th} Compendium of Herbicide Adjuvants, publicly available on the worldwide web (internet) at herbicide.adjuvants.com can be used, e. g., paraffinic oils, polyol fatty acid esters, or oils with short-chain molecules modified with amides or polyamines such as polyethyleneimine or N-pyrrolidine. Transfer agents include, but are not limited to, organosilicone preparations.

In certain embodiments, an organosilicone preparation that is commercially available as Silwet® L-77 surfactant having CAS Number 27306-78-1 and EPA Number: CAL.REG.NO. 5905-50073-AA, and currently available from Momentive Performance Materials, Albany, N.Y. can be used to prepare a polynucleotide composition. In certain embodiments where a Silwet L-77 organosilicone preparation is used as a pre-treatment of plant surfaces, freshly made concentrations in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) are efficacious in preparing a plant surface for transfer of polynucleotide molecules into plant cells from a topical application on the surface. In certain embodiments of the methods and compositions provided herein, a composition that comprises a polynucleotide molecule and an organosilicone preparation comprising Silwet L-77 in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided. In certain embodiments of the methods and compositions provided herein, a composition that comprises a polynucleotide molecule and an organosilicone preparation comprising Silwet L-77 in the range of about 0.3 to about 1 percent by weight (wt percent) or about 0.5 to about 1% by weight (wt percent) is used or provided. In certain embodiments, any of the commercially available organosilicone preparations provided in the following Table 1 can be used as transfer agents in a polynucleotide composition. In certain embodiments where an organosilicone preparation of Table 1 is used as a treatment of plant surfaces, freshly made concentrations in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) are efficacious in preparing a plant surface for transfer of polynucleotide molecules into plant cells from a topical application on the surface. In certain embodiments of the methods and compositions provided herein, a composition that comprises a polynucleotide molecule and an organosilicone preparation of Table 1 in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

TABLE 1

| Name | CAS number | Manufacturer^{1,2} |
|---|---|---|
| BREAK-THRU ® S 321 | na | Evonik Industries AG |
| BREAK-THRU ® S 200 | 67674-67-3 | Evonik Industries AG |
| BREAK-THRU ® OE 441 | 68937-55-3 | Evonik Industries AG |
| BREAK-THRU ® S 278 | 27306-78-1 | Evonik Goldschmidt |
| BREAK-THRU ® S 243 | na | Evonik Industries AG |
| Silwet ® L-77 | 27306-78-1 | Momentive Performance |
| | | Materials |
| Silwet ® HS 429 | na | Momentive Performance |
| | | Materials |
| Silwet ® HS 312 | na | Momentive Performance |
| | | Materials |
| BREAK-THRU ® S 233 | 134180-76-0 | Evonik Industries AG |
| Silwet ® HS 508 | | Momentive Performance |
| | | Materials |
| Silwet ® HS 604 | | Momentive Performance |
| | | Materials |

¹Evonik Industries AG, Essen, Germany
²Momentive Performance Materials, Albany, New York

Organosilicone preparations used in the methods and compositions provided herein can comprise one or more effective organosilicone compounds. As used herein, the phrase “effective organosilicone compound” is used to describe any organosilicone compound that is found in an organosilicone preparation that enables a polynucleotide to enter a plant cell. In certain embodiments, an effective organosilicone compound can enable a polynucleotide to enter a plant cell in a manner permitting a polynucleotide mediated suppression of target gene expression in the plant cell. In general, effective organosilicone compounds include, but are not limited to, compounds that can comprise: i) a trisiloxane head group that is covalently linked to, ii) an alkyl linker including, but not limited to, an n-propyl linker, that is covalently linked to, iii) a poly glycol chain, that is covalently linked to, iv) a terminal group. Trisiloxane head groups of such effective organosilicone compounds include, but are not limited to, heptamethyltrisiloxane. Alkyl linkers can include, but are not limited to, an n-propyl linker. Poly glycol chains include, but are not limited to, polyethylene glycol or polypropylene glycol. Poly glycol chains can comprise a mixture that provides an average chain length “n” of about “7.5”. In certain embodiments, the average chain length “n” can vary from about 5 to about 14. Terminal groups can include, but are not limited to, alkyl groups such as a methyl group. Effective organosilicone compounds are believed to include, but are not limited to, trisiloxane ethoxylate surfactants or polyalkylene oxide modified heptamethyl trisiloxane.

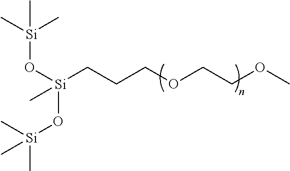

(Compound I: polyalkyleneoxide heptamethyltrisiloxane, average n=7.5).

One organosilicone compound believed to be ineffective comprises the formula:

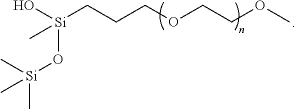

In certain embodiments, an organosilicone preparation that comprises an organosilicone compound comprising a trisiloxane head group is used in the methods and compositions provided herein. In certain embodiments, an organosilicone preparation that comprises an organosilicone compound comprising a heptamethyltrisiloxane head group is used in the methods and compositions provided herein. In certain embodiments, an organosilicone composition that comprises Compound I is used in the methods and compositions provided herein. In certain embodiments, an organosilicone composition that comprises Compound I is used in the methods and compositions provided herein. In certain embodiments of the methods and compositions provided herein, a composition that comprises a polynucleotide molecule and one or more effective organosilicone compound in the range of about 0.015 to about 2 percent by weight (wt percent) (e. g., about 0.01, 0.015, 0.02, 0.025, 0.03, 0.035, 0.04, 0.045, 0.05, 0.055, 0.06, 0.065, 0.07, 0.075, 0.08, 0.085, 0.09, 0.095, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.5 wt percent) is used or provided.

In certain embodiments, the polynucleotide compositions that comprise an organosilicone preparation can comprise a salt such as ammonium chloride, tetrabutylphosphonium bromide, and/or ammonium sulfate. Ammonium chloride, tetrabutylphosphonium bromide, and/or ammonium sulfate can be provided in the polynucleotide composition at a concentration of about 0.5% to about 5% (w/v). An ammonium chloride, tetrabutylphosphonium bromide, and/or ammonium sulfate concentration of about 1% to about 3%, or about 2% (w/v) can also be used in the polynucleotide compositions that comprise an organosilicone preparation. In certain embodiments, the polynucleotide compositions can comprise an ammonium salt at a concentration greater or equal to 300 millimolar. In certain embodiments, the polynucleotide compositions that comprise an organosilicone preparation can comprise ammonium sulfate at concentrations from about 80 to about 1200 mM or about 150 mM to about 600 mM.

In certain embodiments, the polynucleotide compositions can comprise a phosphate salt. Phosphate salts used in the compositions include, but are not limited to, calcium, magnesium, potassium, or sodium phosphate salts. In certain embodiments, the polynucleotide compositions can comprise a phosphate salt at a concentration of at least about 5 millimolar, at least about 10 millimolar, or at least about 20 millimolar. In certain embodiments, the polynucleotide compositions will comprise a phosphate salt in a range of about 1 mM to about 25 mM or in a range of about 5 mM to about 25 mM. In certain embodiments, the polynucleotide compositions can comprise sodium phosphate at a concentration of at least about 5 millimolar, at least about 10 millimolar, or at least about 20 millimolar. In certain embodiments, the polynucleotide compositions can comprise sodium phosphate at a concentration of about 5 millimolar, about 10 millimolar, or about 20 millimolar. In certain embodiments, the polynucleotide compositions will comprise a sodium phosphate salt in a range of about 1 mM to about 25 mM or in a range of about 5 mM to about 25 mM. In certain embodiments, the polynucleotide compositions will comprise a sodium phosphate salt in a range of about 10 mM to about 160 mM or in a range of about 20 mM to about 40 mM. In certain embodiments, the polynucleotide compositions can comprise a sodium phosphate buffer at a pH of about 6.8.

In certain embodiments, other useful transfer agents or adjuvants that can be used in polynucleotide compositions provided herein include surfactants and/or effective molecules contained therein. Surfactants and/or effective molecules contained therein include, but are not limited to, sodium or lithium salts of fatty acids (such as tallow or tallowamines or phospholipids) and organosilicone surfactants. In certain embodiments, the polynucleotide compositions are formulated with counter-ions or other molecules that are known to associate with nucleic acid molecules. Illustrative examples include, tetraalkyl ammonium ions, trialkyl ammonium ions, sulfonium ions, lithium ions, and polyamines such as spermine, spermidine, or putrescine.

In certain embodiments, the polynucleotides used in the compositions that are essentially identical or essentially complementary to the target gene or transcript will comprise the predominant nucleic acid in the composition. Thus in certain embodiments, the polynucleotides that are essentially identical or essentially complementary to the target gene or transcript will comprise at least about 50%, 75%, 95%, 98%, or 100% of the nucleic acids provided in the composition by either mass or molar concentration. However, in certain embodiments, the polynucleotides that are essentially identical or essentially complementary to the target gene or transcript can comprise at least about 1% to about 50%, about 10% to about 50%, about 20% to about 50%, or about 30% to about 50% of the nucleic acids provided in the composition by either mass or molar concentration. Also provided are compositions where the polynucleotides that are essentially identical or essentially complementary to the target gene or transcript can comprise at least about 1% to 100%, about 10% to 100%, about 20% to about 100%, about 30% to about 50%, or about 50% to a 100% of the nucleic acids provided in the composition by either mass or molar concentration.

In certain embodiments, the polynucleotide compositions may comprise glycerin. Glycerin can be provided in the composition at a concentration of about 0.1% to about 1% (w/v or v/v). A glycerin concentration of about 0.4% to about 0.6%, or about 0.5% (w/v or v/v) can also be used in the polynucleotide compositions.

In certain embodiments, the polynucleotide compositions can further comprise organic solvents. Such organic solvents include, but are not limited to, DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions).

In certain embodiments, the polynucleotide compositions can further comprise naturally derived or synthetic oils with or without surfactants or emulsifiers. Such oils include, but are not limited to, plant-sourced oils, crop oils (such as those listed in the 9th Compendium of Herbicide Adjuvants, publicly available on line at www.herbicide.adjuvants.com), paraffinic oils, polyol fatty acid esters, or oils with short-chain molecules modified with amides or polyamines such as polyethyleneimine or N-pyrrolidine.

Compositions and methods of the invention are useful for modulating or suppressing the expression of an endogenous target gene or transgenic target gene in a plant cell or plant. In certain embodiments of the methods and compositions provided herein, expression of EIN2 target genes can be suppressed completely, partially and/or transiently to result in delayed senescence and/or improved appearance. In various embodiments, a target gene includes coding (protein-coding or translatable) sequence, non-coding (non-translatable) sequence, or both coding and non-coding sequence. Compositions of the invention can include polynucleotides and oligonucleotides designed to target multiple genes, or multiple segments of one or more genes. The target gene can include multiple consecutive segments of a target gene, multiple non-consecutive segments of a target gene, multiple alleles of a target gene, or multiple target genes from one or more species. Examples of target genes include endogenous EIN2 genes and EIN2 transgenes.

Target EIN2 genes and plants containing those target EIN2 genes can be obtained from an ornamental plant (e. g., an ornamental flowering plant or shrub or turf grass). Examples of ornamental plants include, but are not limited to, Achillea, Allium, Alstroemeria, Amaryllis, Anemones, Calendula, Calla Lilies, Campanula, Carnation, Celosia, Cosmos, Chrysanthemum, Craspedia Billy Buttons, Crocus, Daffodils, Dahlias, Delphinium, Echinacea, Fall Aster, Freesia, Gardenias, Gerberas, Gerberas Spider, Germini, Gladiolus, Hyacinth, Hydrangea, Hypericum Berry, Iris, Larkspur, Lavender, Lilies, Lily of the Valley, Lisianthus, Lupine, Mums, Orchids, Peonies, Poppy, Ranunculus, Roses, Scabiosa, Snapdragons, Star of Bethlehem, Stephanotis, Sunflowers, Sweet Peas, Sweet William, Tulips, and Zinnia.

An aspect of the invention provides a method for modulating expression of an EIN2 gene in a cut flower by treating the cut flower, or the plant from which the flower is harvested, with one or more polynucleotide molecules, wherein the polynucleotide molecules include at least one segment of 18 or more contiguous nucleotides cloned from or otherwise identified from the target EIN2 gene in either anti-sense or sense orientation, whereby the polynucleotide molecules permeate the interior of the cut flower and induce modulation of the target EIN2 gene. In embodiments of the method, the segment can be cloned or identified from (a) coding (protein-encoding), (b) non-coding (promoter and other gene related molecules), or (c) both coding and non-coding parts of the target EIN2 gene, for example and EIN2 gene of SEQ ID NOs: 1, 4, 6 or 7. Non-coding parts include DNA, such as promoter regions or the RNA transcribed by the DNA that provide RNA regulatory molecules, including but not limited to: introns, 5′ or 3′ untranslated regions, and microRNAs (miRNA), trans-acting siRNAs, natural anti-sense siRNAs, and other small RNAs with regulatory function or RNAs having structural or enzymatic function including but not limited to: ribozymes, ribosomal RNAs, t-RNAs, aptamers, and riboswitches. In certain embodiments where the polynucleotide used in the composition comprises a promoter sequence essentially identical to, or essentially complementary to at least 18 contiguous nucleotides of the promoter of the endogenous target EIN2 gene, the promoter sequence of the polynucleotide is not operably linked to another sequence that is transcribed from the promoter sequence.

Compositions comprising a polynucleotide provided herein can be applied to a cut flower by any convenient method, e.g., spraying or coating with a powder, spraying or coating with a liquid composition, or by providing in the water taken up by the cut flower. Topically applied sprays or coatings can be of either all or of any a portion of the surface of the cut flower or the plant from which the flower is harvested, prior to harvest.

Several embodiments relate to a watering solution comprising a polynucleotide comprising a sequence homologous to a target gene as described herein for extending the storage life of cut flowers compared to a cut flower placed in water alone. In some embodiments, the polynucleotide is a dsRNA molecule comprising at least a 15-22 nucleotide sequence which is homologous to a target gene. In some embodiments, the target gene is a rose EIN2 gene. In some embodiments, the target gene is a carnation EIN2 gene. In some embodiments, the watering solution further comprises a transfer agent, for example, an organosilicone transfer agent. In some embodiments, the watering solution further comprises one or more of nutrients, water uptake stimulants, and chemical preservatives. Examples of nutrients include carbohydrates such as sucrose, fructose, glucose, lactose and maltose. Examples of water uptake stimulants include acidulants, such as citric acid, glycolic acid, malic acid and aluminium sulphate, and anionic and non-ionic surfactants. Examples of chemical preservatives include biocides, such as isothiazolinones, bronopol and quaternary ammonium salts. Examples of biocides include fungicides, antibiotics, bactericides, and yeast inhibitors.

Another embodiment relates to a method of putting cut flowers into vase water, said method comprising immersing the stems of one or more cut flowers into vase water and adding a composition comprising a polynucleotide comprising a sequence homologous to a target gene as described herein to the vase water before, after or at the same time as the cut flowers are immersed into the vase water. In some embodiments, the polynucleotide is a dsRNA molecule comprising at least a 15-22 nucleotide sequence which is homologous to a target gene. In some embodiments, the target gene is a rose EIN2 gene. In some embodiments, the target gene is a carnation EIN2 gene. In some embodiments, a transfer agent, for example, an organosilicone transfer agent is added to the vase water. In some embodiments, one or more of nutrients, water uptake stimulants, fungicides, and chemical preservatives is added to the vase water. Examples of nutrients include carbohydrates such as sucrose, fructose, glucose, lactose and maltose. Examples of water uptake stimulants include acidulants, such as citric acid, glycolic acid, malic acid and aluminium sulphate, and anionic and non-ionic surfactants. Examples of chemical preservatives include biocides, such as isothiazolinones, bronopol and quaternary ammonium salts. Examples of biocides include fungicides, antibiotics, bactericides, and yeast inhibitors.

The composition comprising the polynucleotide may be added to the vase water in the form of a tablet, a powder, a paste or of a fluid having a dry matter content of at least 5 g/l. In some embodiments, the composition is added in the form of a tablet or a powder. When in the form of dry powders, compositions comprising a polynucleotide as described herein are suitably packaged in bulk for end use, as in containers having a tightly-fitting lid such as screw-capped or snap-capped bottles or, are packaged in plastic, foil or paper sachets containing the required amount of material for a single use. Effervescent ingredients may be incorporated to accelerate dispersion and dissolving of the composition.

In some embodiments, a composition comprising a polynucleotide as described herein may be provided in a kit comprising a polynucleotide composition and one or more cut flowers. In some embodiments, the polynucleotide composition may be provided in the form of a tablet, a powder, a paste or of a fluid, which may be packaged in single-use container having a tightly-fitting lid, such as screw-capped or snap-capped bottles, or packaged in plastic, foil or paper sachets. The kit may further comprise one or more of a transfer agent, nutrients, water uptake stimulants, and chemical preservatives, which may either be provided in the polynucleotide composition, or packaged separately. In some embodiments, the kit further comprises a vase.

### Example 1

### Polynucleotide Treatment of Carnation Flowers with Triggers Homologous to the EIN2 Gene

Polynucleotide triggers (0.5-2 nmol) homologous to the carnation EIN2 gene were prepared in 0.01% Silwet L-77 in a total volume of 1 mL. Flowers stems were cut to 25 cm under dH20 and then transferred to 15 ml conical polypropylene tubes containing the trigger-Silwet solution (Day 0). After approximately 75% uptake of the trigger solution, an additional 1 mL of H20 was added to each tube and again allowed to be absorbed. Each tube was then filled to the 15 mL mark and allowed to stand overnight in a 25 C growth chamber with 16 h light. Treatments were randomized in the racks.

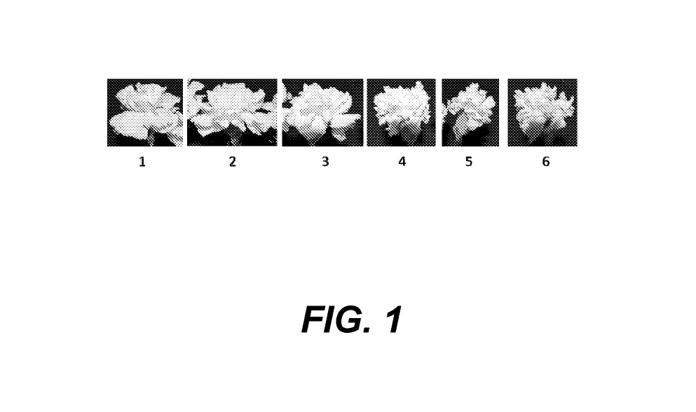

On the following day (Day 1), the flowers were transferred to new tubes, arrayed in identical order, containing 12 mL dH20. Between Day 1 and Day 4 tubes were monitored for signs of senescence and refilled with dH2O and 100 mg/L 8-Hydroxyquinoline sulfate (8-HQS) as needed. From Day 4 until the termination of the experiment, tubes were refilled every third day with dH2O and 100 mg/L 8-HQS and daily measurements of senescence were taken. Flower diameters were measured at days 6, 15 and 18 of the study. Senescence ratings from 1 to 6 (FIG. 1) were assigned to plants on day 18. There were 5 trigger treatments that consisted of pools of three triggers each (Table 2). Each of those treatments plus a non-specific trigger control were tested at 3 dose levels. There were 8 replicates at the two lower dose levels and five replicates at the high dose.

TABLE 2
Triggers used in carnation experiments. Sense and antisense
sequences were annealed to produce the double stranded RNA
trigger. ″Pool″ indicates triggers combined in treatments.
Control sequences were generated using a bioinformatics
process such that they would not match to any available
sequences in soybean, tomato, cucumber, lettuce, cotton or
corn with identity over 94.7%.

| Sequence Name | Actual Sequence | SEQ ID | Pool |
|---|---|---|---|
| Control | GCCGUAGCGAGCAUACGUAUG | 59231_8 | |
| ControlAntisense | UACGUAUGCUCGCUACCGGCGC | 59231_9 | |
| T25895 | UUCUCGUGCUGCUGUUAGAAU | 59231_10 | 1 |
| T25895Antisense | UCUAACAGCAGCACGAUGAAUC | 59231_11 | 1 |
| T25896 | CAGAUAGAAGCGGCGUAUAAG | 59231_12 | 1 |
| T25896Antisense | UAUACGCCGCUUCUAUACUGUC | 59231_13 | 1 |
| T25897 | GAGGCGUGGUCUCAAGAUAAU | 59231_14 | 1 |
| T25897Antisense | UAUCUUGAGACCACGCACUCGU | 59231_15 | 1 |
| T25898 | AGGGUUGGUUUGCUAUCUAUC | 59231_16 | 2 |
| T25898Antisense | UAGAUAGCAAACCAACACCUGU | 59231_17 | 2 |
| T25899 | GGAAGUGAAUGGGUCGUUAAC | 59231_18 | 2 |
| T25899Antisense | UAACGACCCAUUCACUCUCCCC | 59231_19 | 2 |
| T25900 | GACCAGUUCAGGAGCUUUACG | 59231_20 | 2 |
| T25900Antisense | UAAAGCUCCUGAACUGUGUCCA | 59231_21 | 2 |
| T25901 | AUCAGUAACGCGGUAAACAAU | 59231_22 | 3 |
| T25901Antisense | UGUUUACCGCGUUACUUGAUUU | 59231_23 | 3 |
| T25902 | UCAGAAGCAAGGAGUAAGAAA | 59231_24 | 3 |
| T25902Antisense | UCUUACUCCUUGCUUCUUGAGU | 59231_25 | 3 |
| T25903 | ACCCAGUCUUCUUGAUUCAGG | 59231_26 | 3 |
| T25903Antisense | UGAAUCAAGAAGACUGCGGUUA | 59231_27 | 3 |
| T25904 | AGAGCGGUAUCAUAGUGUACG | 59231_28 | 4 |
| T25904Antisense | UACACUAUGAUACCGCUUCUCC | 59231_29 | 4 |
| T25905 | UUGCGAGAUUGAGAGCAAAUU | 59231_30 | 4 |
| T25905Antisense | UUUGCUCUCAAUCUCGGCAAAC | 59231_31 | 4 |
| T25906 | UUUAAACCUCGGACGUCAAUG | 59231_32 | 4 |
| T25906Antisense | UUGACGUCCGAGGUUUGAAAAA | 59231_33 | 4 |
| T25907 | GGAAGGGUUGCGUUUGGAAGU | 59231_34 | 5 |
| T25907Antisense | UUCCAAACGCAACCCUCUCCAC | 59231_35 | 5 |
| T25908 | GGCUUUUUCAAACCUCGGACG | 59231_36 | 5 |
| T25908Antisense | UCCGAGGUUUGAAAAACGCCAA | 59231_37 | 5 |
| T25909 | CCCCUGCUUCUGCCUCCAAGU | 59231_38 | 5 |
| T25909Antisense | UUGGAGGCAGAAGCAGGGGGAC | 59231_39 | 5 |

Flowers treated with trigger Pool 2 had a significantly greater mean flower diameter than flowers treated with the control trigger at 18 days with a 0.1 nmol/stem dose (Table 3), indicating a decrease in flower senescence. No improvements in flower diameter versus the control were found on earlier measurement days or with other doses (data not shown). When analyzed across dose levels, the mean diameter of flowers treated with trigger Pool 2 was also significantly greater than control flowers at day 18 (Table 4).

TABLE 3
Mean flower diameter (mm) at days 6, 15 and 18 for flowers
treated with 0.1 nmol trigger, and the rate of change
of flower diameter from day 6 to day 18 (slope). Letters
indicate significant differences, p < 0.05.

| Dose | Trigger | Day 6* | Day 15* | Day 18* | Slope* |
|---|---|---|---|---|---|
| 0.1 | Pool 1 | 74.5 | 57.8 | 51.1 | −1.92 |
| 0.1 | Pool 2 | 68.1 | 64.8 | 61.9a | −0.49a |
| 0.1 | Pool 3 | 78.1 | 52.4 | 47.5 | −2.62 |
| 0.1 | Pool 4 | 79.4 | 64.6 | 53.8 | −2.02 |
| 0.1 | Pool 5 | 67.9 | 58.9 | 51.3 | −1.29 |
| 0.1 | Control | 73.1 | 56.3 | 48.8b | −1.99b |
| | P-value | 0.156 | 0.037 | 0.002 | 0.021 |
| | LSD for Control | 11.2 | 13.2 | 10.1 | |
| | vs Trigger | | | | |
| | (P < 0.05) | | | | |
| | LSD for Control | 9.3 | 11.0 | 8.5 | |
| | vs Trigger | | | | |
| | (P < 0.10) | | | | |

*Mean separation only noted for difference from Control.

TABLE 4
Mean flower diameter (mm) at days 6, 15 and 18 analyzed across
doses. Letters indicate significant differences, p < 0.05.

| Dose | Trigger | Day 6* | Day 15* | Day 18* |
|---|---|---|---|---|
| All | Pool 1 | 76.9 | 57.3 | 50.5 |
| All | Pool 2 | 75.1 | 63.5 | 58.1a |
| All | Pool 3 | 77.4 | 54.0 | 49.5 |
| All | Pool 4 | 76.4 | 59.6 | 50.7 |
| All | Pool 5 | 70.6 | 54.1 | 48.9 |
| All | Control | 74.7 | 59.5 | 51.1b |
| | LSD for Control | 5.8 | 8.2 | 6.4 |
| | vs Trigger | | | |
| | (P < 0.05) | | | |
| | LSD for Control | 4.9 | 6.8 | 5.3 |
| | vs Trigger | | | |
| | (P < 0.10) | | | |

*Mean separation only noted for difference from Control.

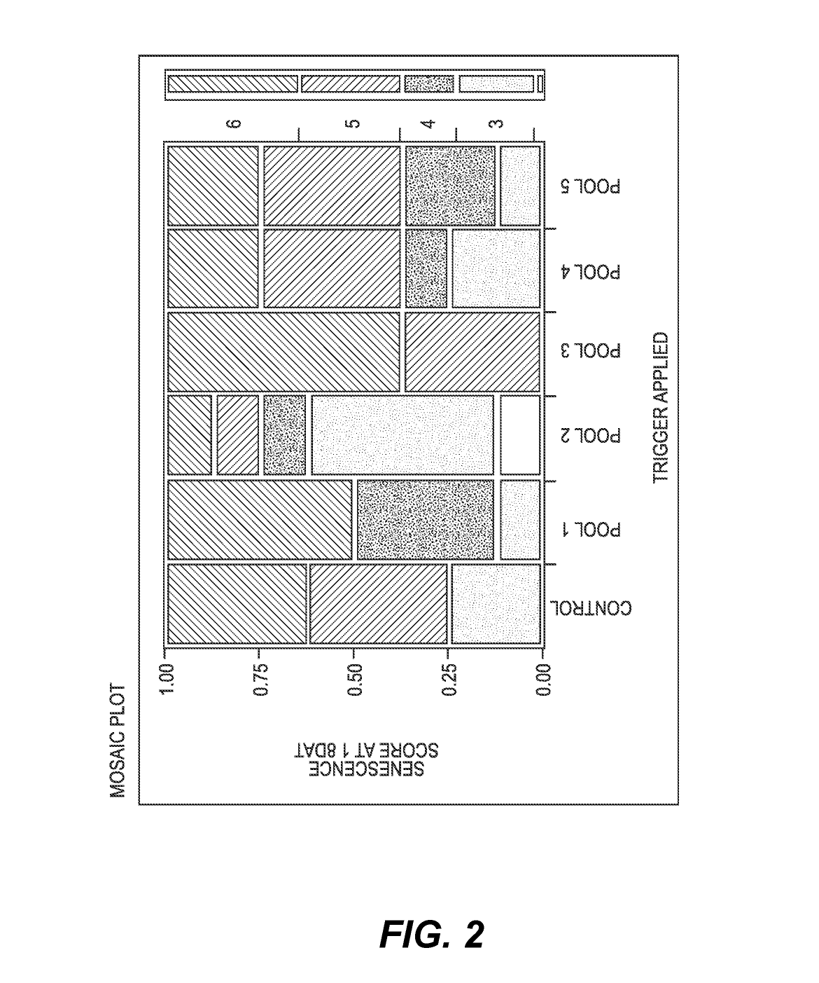

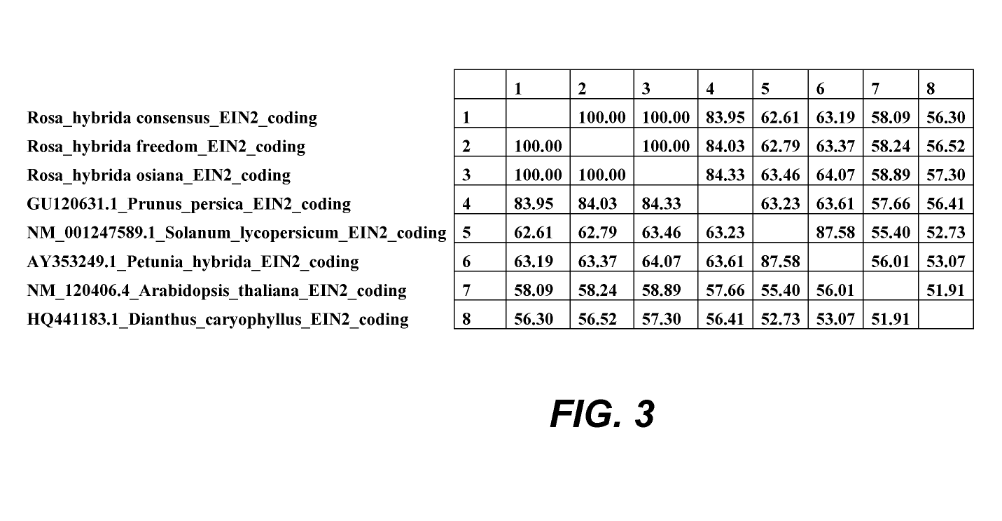

Senescence scores were significantly improved for flowers treated with the trigger Pool 2 versus the Control at the low dose and also when the data were analyzed across dose levels (Table 5). Approximately 62% of flowers treated with Pool 2 triggers had senescence scores of 3 or less, compared to only 25% of flowers treated with the control trigger (FIG. 2).

TABLE 5
Analysis of flower senescence by dose and across doses at
day 18. The numbers in the table are P-values versus the
non-specific Control trigger. The direction of the effect
is indicated in parentheses for significant (P < 0.10)
responses. ‘+’ indicates delayed senescence.

| | 0.1 nmol/ | 1.0 nmol/ | 2.0 nmol/ | Combined across |
|---|---|---|---|---|
| Trigger | stem | stem | stem | doses |
| Pool 1 | 0.948 | 0.745 | 0.605 | 0.857 |
| Pool 2 | 0.036(+) | 0.155 | 0.427 | 0.010(+) |
| Pool 3 | 0.222 | 0.703 | 0.536 | 0.174 |
| Pool 4 | 0.640 | 0.745 | 0.536 | 0.928 |
| Pool 5 | 0.761 | 0.229 | 0.708 | 0.792 |

Thus, flowers treated with trigger Pool 2 at a dose of 0.1 nmol showed a statistically significant decrease in flower senescence compared to control flowers using 2 measures. They had larger diameters (Tables 3 and 4), and they showed less senescence using a visual score (Table 5 and FIG. 2) at 18 days after treatment.

To confirm the effect of trigger Pool 2 on delaying carnation flower senescence, a second experiment was conducted comparing the effects of this trigger pool with a control trigger. In this experiment, 4 trigger doses were applied (0.01, 0.1, 1 and 2 nmol/stem) as described above. Flowers were randomized in the growth chamber. The visual senescence score (FIG. 1) was measured 7 times between days 6-15, and the EIN2 trigger Pool 2 was compared with the non-specific trigger for each dose (Table 6). A reduction in flower senescence was observed at the 0.1 nmol dose at days 12, 13 and 14 and at 1 nmol trigger for day 12. At day 12, 95% of flowers treated with control trigger had a senescence score of 4 or more, but only 75% of flowers treated with trigger Pool 2 had senesced to this level (data not shown). These results confirm the effects of EIN2 trigger Pool 2 on reducing senescence of carnation flowers.

TABLE 6
Comparison of treatment with EIN2 trigger Pool 2 with the control
trigger on flower senescence. The numbers in the table represent
P-values for the difference of mean senescence values for flowers
treated with trigger Pool 2 versus the Control non-specific trigger.
In parentheses is the direction of the effect with ‘+’
indicating reduced flower senescence, and ‘−’ indicating
more advanced flower senescence compared to the control.

| | Day of Study | | | | | | | |
|---|---|---|---|---|---|---|---|---|
| Dose | 6 | 7 | 8 | 11 | 12 | 13 | 14 | 15 |
| 0.01 | 0.090 | 0.109 | 0.436 | 0.430 | 0.386 | 0.562 | 0.842 | 0.566 |
| | (−) | | | | | | | |
| 0.1 | 0.294 | 0.340 | 0.123 | 0.315 | 0.063 | 0.073 | 0.076 | 0.153 |
| | | | | | (+) | (+) | (+) | |
| 1.0 | 0.235 | 0.946 | 0.121 | 0.142 | 0.055 | 0.116 | 0.116 | 0.116 |
| | | | | | (+) | | | |
| 2.0 | 0.271 | 0.253 | 0.636 | 0.575 | 0.658 | 0.807 | 0.940 | 0.829 |

### Example 2

### Polynucleotide Treatment of Rose Flowers with Triggers Homologous to the EIN2 Gene

Polynucleotide triggers (0.5-2 nmol) homologous to the rose EIN2 gene are prepared in 0.01% Silwet L-77 in a total volume of 1 mL. Rose flowers are obtained from a commercial grower where stems are harvested and shipped under normal commercial handling for each variety, most commonly at the tight-bud stage. Flowers typically arrive 4-5 days postharvest at which time flower stems are cut to 25 cm under diH20 and then transferred to 15 ml conical polypropylene tubes containing the trigger-Silwet solution. After approximately 75% uptake of the trigger solution, an additional 1 mL of diH20 is added to each tube and is allowed to be taken up. Each tube is then filled to the 15 mL mark with diH2O and allowed to stand overnight in a 25 C growth chamber with 16 h light. Treatments are randomized in the racks. On the following day, the flowers are dipped in diH20 to wash off residual trigger solution and transferred to new tubes, arrayed in identical order, containing 12 mL dH20. Flower diameters and degree of bending of the stem are measured every day. In addition, a visual score is recorded for each flower. The scoring system is:

Best—(Pass)—flower without blemish, fully open, and highly turgid

Keep—(Pass)—flower with minor blemish, fully open, acceptable turgidity

Poor—(Pass)—flower significantly blemished, not fully open, less than full turgidity but not wilted

Wilt—(Fail)—petals showing signs of lost turgidity (wrinkled surface, color change)

Fail—(Fail)—petals senesced

TABLE 7
Rose Triggers used in carnation experiments. Sense and antisense
sequences are annealed to produce a double stranded RNA trigger.
Control sequences were generated using a bioinformatics process such
that they would not match to any available sequences in soybean,
tomato, cucumber, lettuce, cotton or corn with identity over 94.7%.

| | | | % homology to |
|---|---|---|---|
| Sequence Name | Actual Sequence | SEQ ID | Carnation |
| Control | GCCGUAGCGAGCAUACGUAUG | 59231_8 | 0 |
| ControlAntisense | UACGUAUGCUCGCUACCGGCGC | 59231_9 | |
| T25930 | CUUACGUGGUUUGCUCACAUU | 59231_40 | 60.8 |
| T25930Antisense | UGUGAGCAAACCACGUGAAGUG | 59231_41 | |
| T25931 | GAAAGUGAUUGGGUGGAUAAU | 59231_42 | 75 |
| T25831Antisense | UAUCCACCCAAUCACUAUUCCC | 59231_43 | |
| T25932 | GAGCUGAUCAGGAGCUUCAAU | 59231_44 | 70.8 |
| T25932Antisense | UGAAGCUCCUGAUCAGGCUCCA | 59231_45 | |

The number of days to the Wilt stage is recorded as the vase life. Triggers which provide a statistically significant decrease in rose flower senescence compared to control (untreated or control dsRNA treated) rose flowers are selected for use as a floral preservative.

### Example 3

### Identification of Rose Ethylene Signaling and Biosynthetic Pathway Sequences

This example describes non-limiting embodiments of methods for identifying rose ethylene signaling and biosynthetic pathway coding sequences, useful in engineering polynucleotides which prevent or reduce rose senescence.

Rose cDNA orthologs to the known sequences of Arabidopsis thaliana ethylene signaling and biosynthetic pathway genes (EIN2, EIN3, ACC synthase (ACS), ACC oxidase (ACO)) were identified using Tblastx. Arabidopsis nucleotide sequences for EIN2, EIN3, ACS, and ACO were compared (e value E-05) to a unigene library for the Rosa hybrid, osiana and orthologous sequences were identified. The orthologous sequences identified in this analysis were confirmed by performing a reciprocal Blast against Arabidopsis thaliana (TAR 9.0). Table 8 shows the result of this analysis.

TABLE 8
Rose cDNA orthologs to known Arabidopsis thaliana ethylene signaling
and biosynthetic genes.

| SEQ ID NO | Sequence_ID | Annotation | Type | Species |
|---|---|---|---|---|
| 46 | ROSHY_OS-26SEP13- | EIN2 | DNA | Rosa hybrid |
| | TRPT0045858 | | | |
| 47 | ROSHY_OS-26SEP13- | EIL1 | DNA | Rosa hybrid |
| | TRPT0034544 | | | |
| 48 | ROSHY_OS-26SEP13- | ETHYLENE-INSENSITIVE3 | DNA | Rosa hybrid |
| | TRPT0026603 | protein | | |
| 49 | ROSHY_OS-26SEP13- | EIL2 | DNA | Rosa hybrid |
| | TRPT0039394 | | | |
| 50 | ROSHY_OS-26SEP13- | EIL2 | DNA | Rosa hybrid |
| | TRPT0039393 | | | |
| 51 | ROSHY_OS-26SEP13- | ACS | DNA | Rosa hybrid |
| | TRPT0042476 | | | |
| 52 | ROSHY_OS-26SEP13- | ACS | DNA | Rosa hybrid |
| | TRPT0034045 | | | |
| 53 | ROSHY_OS-26SEP13- | ACS | DNA | Rosa hybrid |
| | TRPT0013256 | | | |
| 54 | ROSHY_OS-26SEP13- | ACS | DNA | Rosa hybrid |
| | TRPT0052979 | | | |
| 55 | ROSHY_OS-26SEP13- | 1-aminocyclopropane- | DNA | Rosa hybrid |
| | TRPT0006340 | 1-carboxylate oxidase | | |
| 56 | ROSHY_OS-26SEP13- | 1-aminocyclopropane- | DNA | Rosa hybrid |
| | TRPT0060679 | 1-carboxylate oxidase | | |
| 57 | AAM20924 | Rh-EIN3-1 | DNA | Rosa hybrid |
| 58 | AAY15109 | Rh-EIN3-2 | DNA | Rosa hybrid |
| 59 | AAL14267.1 | EIN3-like | DNA | Rosa hybrid |
| 60 | AF441282 | Rh-ACO1 | DNA | Rosa hybrid |
| 61 | AY525069 | ACS5 | DNA | Rosa hybrid |
| 62 | AY525068 | ACS4 | DNA | Rosa hybrid |
| 63 | AY525067 | ACS3 | DNA | Rosa hybrid |
| 64 | AY525066 | ACS2 | DNA | Rosa hybrid |
| 65 | AY803737 | Rh-ACS2 | DNA | Rosa hybrid |
| 66 | AY061946.1 | Rh-ACS1 | DNA | Rosa hybrid |

### Example 4

### Treatment of Rose Flowers with Polynucleotide Triggers Targeting Ethylene Signaling and Biosynthetic Pathway Genes

Double stranded RNA triggers comprising at least 18 contiguous nucleotides that are essentially identical or essentially complementary to a rose cDNA ortholog of EIN2, EIL1, Ethylene-Insensitive 3, EIL2, ACS, 1-Aminocyclopropane-1-Carboxylate Oxidase, Rh-EIN3-1, Rh-EIN3-2, Rh-ACO1, ACS5, ACS4, ACS3, ACS2 and Rh-ACS2 are generated. The polynucleotide triggers (0.5-2 nmol) are prepared in 0.01% Silwet L-77 in a total volume of 1 mL. Rose flowers are obtained from a commercial grower where stems are harvested and shipped under normal commercial handling for each variety, most commonly at the tight-bud stage. Flowers typically arrive 4-5 days post-harvest at which time flower stems are cut to 25 cm under diH20 and then transferred to 15 ml conical polypropylene tubes containing the trigger-Silwet solution. After approximately 75% uptake of the trigger solution, an additional 1 mL of diH20 is added to each tube and is allowed to be taken up by the flower. Each tube is then filled to the 15 mL mark with diH2O and allowed to stand overnight in a 25° C. growth chamber with 16 h light. Treatments are randomized in the racks. On the following day, the flowers are dipped in diH20 to wash off residual trigger solution and transferred to new tubes, arrayed in identical order, containing 12 mL dH20. Flower senescence (browning, wilting, flower diameter, ets.) is measured every day and a visual senescence score is assigned to the treated flowers. Triggers which delay or minimize floral senescence are selected.

### Example 5

### Identification of Rose Genes Expressed During Petal Senescence

RNA sequencing (RNA-seq) was performed to analyze the abundance of specific rose mRNAs expressed before and during petal senescence. RNA was extracted from rose petal tissue at different time points from unopened bud through senescent flower. The following time points were compared: T2 (fresh opened flower) vs T4 (flower with hints of senescence) and T2 (fresh opened flower) vs T8 (fully senescing flower). The analysis of T2 (fresh opened flower) vs T8 (fully senescing flower) revealed a number of transcription factors up-regulated in the senescing flower that were not present in the fresh opened flower bud. These are summarized in Table 9.

TABLE 9
Regulatory genes highly represented in RNA sequencing analysis between fresh and senescing flowers.

| SEQ ID NO | SmartBlastAnnotation |
|---|---|
| 67 | NAC domain-containing protein, putative n = 1 Tax = Ricinus communis RepID = B9SQZ6_RICCO; |
| | exp = 8e−69 |
| 68 | GRAS family transcription factor n = 1 Tax = Populus trichocarpa RepID = B9IGF1_POPTR; exp = 1e−68 |
| 69 | Homeobox leucine zipper protein n = 1 Tax = Prunus armeniaca RepID = Q9XH73_PRUAR; exp = 3e−56 |
| 70 | NAC domain-containing protein 21/22, putative n = 1 Tax = Ricinus communis |
| | RepID = B9RLW7_RICCO; exp = 6e−31 |
| 71 | GRAS family transcription factor n = 1 Tax = Populus trichocarpa RepID = B9IGF1_POPTR; exp = 1e−131 |
| 72 | Auxin-responsive protein IAA20, putative n = 1 Tax = Ricinus communis RepID = B9RN35_RICCO; |
| | exp = 5e−33 |
| 73 | R2r3-myb transcription factor, putative n = 1 Tax = Ricinus communis RepID = B9SYQ1_RICCO; |
| | exp = 7e−45 |
| 74 | NAC domain-containing protein, putative n = 1 Tax = Ricinus communis RepID = B9S8Z7_RICCO; |
| | exp = 1e−107 |
| 75 | Putative basic helix-loop-helix protein BHLH2 n = 1 Tax = Lotus japonicus RepID = COJP10_LOTJA; |
| | exp = 4e−50 |
| 76 | WRKY transcription factor, putative n = 1 Tax = Ricinus communis RepID = B9S164_RICCO; exp = 1e−51 |
| 77 | Nuclear transcription factor Y subunit A-1, putative n = 1 Tax = Ricinus communis |
| | RepID = B9RID0_RICCO; exp = 8e−36 |
| 78 | WRKY transcription factor, putative n = 1 Tax = Ricinus communis RepID = B9S8C8_RICCO; exp = 3e−66 |
| 79 | Polycomb group protein EMF2 n = 1 Tax = Asparagus officinalis RepID = Q1W6K9_ASPOF; exp = 1e−44 |
| 80 | Homeobox protein, putative n = 1 Tax = Ricinus communis RepID = B9SVE1_RICCO; exp = 2e−65 |
| 81 | GHMYB10 n = 1 Tax = Gossypium hirsutum RepID = Q9ATD5_GOSHI; exp = 2e−67 |
| 82 | SIN3 component, histone deacetylase complex n = 1 Tax = Populus trichocarpa |
| | RepID = B9HU88_POPTR; exp = 3e−50 |
| 83 | Transcription factor, putative n = 1 Tax = Ricinus communis RepID = B9RWH6_RICCO; exp = 1e−110 |
| 84 | AP2/ERF domain-containing transcription factor n = 1 Tax = Populus trichocarpa |
| | RepID = B9GJI7_POPTR; exp = 2e−35 |
| 85 | NAC domain protein NAC6 n = 2 Tax = Glycine max RepID = Q52QR0_SOYBN; exp = 4e−70 |
| 86 | AP2 domain-containing transcription factor n = 2 Tax = Populus trichocarpa |
| | RepID = B9N303_POPTR; exp = 2e−48 |
| 87 | Dam2 n = 4 Tax = Prunus persica RepID = A6XN00_PRUPE; exp = 3e−55 |
| 88 | Trihelix transcription factor n = 1 Tax = Glycine max RepID = B0EW03_SOYBN; exp = 1e−20 |
| 89 | NAC domain protein n = 1 Tax = Citrus trifoliata RepID = COKLH1_PONTR; exp = 1e−122 |
| 90 | WRKY 13 (Fragment) n = 1 Tax = Theobroma cacao RepID = Q6VR10_THECC; exp = 2e−96 |
| 91 | Transcription factor, putative n = 1 Tax = Ricinus communis RepID = B9RHS2_RICCO; exp = 1e−117 |
| 92 | MADS-box protein AGL15 (Fragment) n = 1 Tax = Dimocarpus longan RepID = D4P8F4_9ROSI; |
| | exp = 1e−47 |
| 93 | MADS9 protein n = 1 Tax = Gossypium hirsutum RepID = Q5Y9B9_GOSHI; exp = 5e−77 |
| 94 | GRAS family transcription factor n = 2 Tax = Populus trichocarpa RepID = B9GTP1_POPTR; exp = 0 |
| 95 | EIL1 n = 1 Tax = Prunus persica RepID = A0MQ93_PRUPE; exp = 0 |
| 96 | Auxin response factor, putative n = 1 Tax = Ricinus communis RepID = B9R865_RICCO; exp = 0 |
| 97 | Auxin response factor, putative n = 1 Tax = Ricinus communis RepID = B9R865_RICCO; exp = 0 |
| 98 | IAA-amino acid hydrolase ILR1, putative n = 1 Tax = Ricinus communis RepID = B9S5P0_RICCO; |
| | exp = 1e−165 |
| 99 | AP2/ERF domain-containing transcription factor n = 1 Tax = Populus trichocarpa |
| | RepID = B9G221_POPTR; exp = 7e−43 |
| 100 | AP2/ERF domain-containing transcription factor n = 1 Tax = Populus trichocarpa |
| | RepID = B9G221_POPTR; exp = 7e−43 |
| 101 | Nuclear transcription factor Y subunit A-1, putative n = 1 Tax = Ricinus communis |
| | RepID = B9RVQ7_RICCO; exp = 1e−84 |
| 102 | WRKY DNA binding protein n = 1 Tax = Fragaria x ananassa RepID = C7S811_FRAAN; exp = 1e−76 |
| 103 | Protein AINTEGUMENTA, putative n = 1 Tax = Ricinus communis RepID = B9SW78_RICCO; exp = 1e−127 |
| 104 | Basic helix-loop-helix-containing protein, putative n = 1 Tax = Ricinus communis |
| | RepID = B9T627_RICCO; exp = 2e−47 |
| 105 | NAC domain-containing protein 21/22, putative n = 1 Tax = Ricinus communis |
| | RepID = B9SVD5_RICCO; exp = 1e−105 |
| 106 | Auxin-responsive protein IAA6, putative n = 1 Tax = Ricinus communis RepID = B9RQE0_RICCO; |
| | exp = 8e−97 |
| 107 | Auxin-responsive protein IAA6, putative n = 1 Tax = Ricinus communis RepID = B9RQE0_RICCO; |
| | exp = 8e−92 |
| 108 | Ethylene-responsive transcription factor, putative n = 1 Tax = Ricinus communis |
| | RepID = B9RIM8_RICCO; exp = 2e−26 |
| 109 | Mads box protein, putative n = 1 Tax = Ricinus communis RepID = B9RFR5_RICCO; exp = 3e−25 |
| 110 | Transcription factor MYB251 n = 1 Tax = Fagus crenata RepID = B5UAQ2_FAGCR; exp = 8e−76 |
| 111 | Ocs element-binding factor, putative n = 1 Tax = Ricinus communis RepID = B9RH86_RICCO; |
| | exp = 5e−54 |

### Example 6

### Polynucleotide Treatment of Rose Flowers with Triggers Identified Through RNA Sequencing

The regulatory genes identified in Table 9 as up-regulated in senescing flowers are used as the basis for selecting sequences for floral preservation studies. Double stranded RNA triggers comprising at least 18 contiguous nucleotides that are essentially identical or essentially complementary to a gene identified in Table 9 are generated. The polynucleotide triggers (0.5-2 nmol) are prepared in 0.01% Silwet L-77 in a total volume of 1 mL. Rose flowers are obtained from a commercial grower where stems are harvested and shipped under normal commercial handling for each variety, most commonly at the tight-bud stage. Flowers typically arrive 4-5 days post-harvest at which time flower stems are cut to 25 cm under diH20 and then transferred to 15 ml conical polypropylene tubes containing the trigger-Silwet solution. After approximately 75% uptake of the trigger solution, an additional 1 mL of diH20 is added to each tube and is allowed to be taken up by the flower. Each tube is then filled to the 15 mL mark with diH2O and allowed to stand overnight in a 25° C. growth chamber with 16 h light. Treatments are randomized in the racks. On the following day, the flowers are dipped in diH20 to wash off residual trigger solution and transferred to new tubes, arrayed in identical order, containing 12 mL dH20. Flower senescence (browning, wilting, flower diameter, etc.) is measured every day and a visual senescence score is assigned to the treated flowers. Genes which delay or minimize floral senescence when suppressed are selected. Additional trigger polynucleotides are designed which target the selected genes are designed and tested for efficacy in delaying floral senescence.

What is claimed is:

1. A method for delaying senescence in a cut flower, comprising topically applying to a plant surface a composition that comprises at least one polynucleotide that comprises at least 18 contiguous nucleotides that are essentially identical or essentially complementary to an EIN2 gene or to a transcript of said gene, wherein the cut flower exhibits delayed senescence that results from suppression of the EIN2 gene.

2. The method of claim 1, wherein the composition further comprises a transfer agent.

3. The method of claim 2, wherein the transfer agent comprises an organosilicone preparation.

4. The method of any one of claims 1-3, wherein the polynucleotide molecule comprises sense ssDNA, sense ssRNA, dsRNA, dsDNA, a double stranded DNA/RNA hybrid, anti-sense ssDNA, or anti-sense ssRNA.

5. The method of any one of claims 1-4, wherein said polynucleotide is selected from the group consisting of SEQ ID NOs: 8-39, or wherein said polynucleotide comprises at least 18 contiguous nucleotides that are essentially identical or essentially complementary to SEQ ID NO: 4.

6. The method of claim 5, wherein said polynucleotide is selected from the group consisting of SEQ ID NOs: 16-21.

7. The method of any one of claims 1-4, wherein said polynucleotide is selected from the group consisting of SEQ ID NOs: 40-45, or wherein said polynucleotide comprises at least 18 contiguous nucleotides that are essentially identical or essentially complementary to SEQ ID NO: 1 or 7.

8. The method of any one of claims 1-7, wherein;
(a) the cut flower is a carnation, the gene or the transcript is a carnation EIN2 gene or transcript, and said polynucleotide molecule is selected from the group consisting SEQ ID NO: 8-39 or said polynucleotide comprises at least 18 contiguous nucleotides that are essentially identical or essentially complementary to SEQ ID NO: 4; or
(b) the cut flower is a rose, the gene or the transcript is a rose EIN2 gene or transcript, and said polynucleotide molecule is selected from the group consisting of SEQ ID NO: 40-45 or said polynucleotide comprises at least 18 contiguous nucleotides that are essentially identical or essentially complementary to SEQ ID NOs: 1 or 7.

9. The method of any one of claims 1-8, wherein said composition comprises any combination of two or more polynucleotide molecules.

10. The method of any one of claims 1-8, wherein the composition is applied to a cut or exposed surface of the flower stem.

11. A composition for extending the vase life of cut flowers, comprising: a polynucleotide molecule that comprises at least 18 contiguous nucleotides that are essentially identical or essentially complementary to a EIN2 gene or transcript of said gene, wherein said polynucleotide comprises sense ssDNA, sense ssRNA, dsRNA, dsDNA, a double stranded DNA/RNA hybrid, anti-sense ssDNA, or anti-sense ssRNA.

12. The composition of claim 11, wherein the composition further comprises a transfer agent.

13. The composition of claim 12, wherein the transfer agent comprises an organosilicone preparation.

14. The composition of any one of claims 11-13, wherein the polynucleotide is selected from the group consisting of SEQ ID NO: 8-39, or wherein said polynucleotide comprises at least 18 contiguous nucleotides that are essentially identical or essentially complementary to SEQ ID NO:4.

15. The composition of any one of claims 11-13, wherein the polynucleotide is selected from the group consisting of SEQ ID NO: 40-45, or wherein said polynucleotide comprises at least 18 contiguous nucleotides that are essentially identical or essentially complementary to SEQ ID NO: 1 or 7.

16. The composition of any one of claims 11-15, wherein the composition further comprises any combination of two or more polynucleotide molecules.

17. The composition of any one of claims 11-16, wherein the composition further comprises one or more of a transfer agent, nutrients, water uptake stimulants, and chemical preservatives.

18. A kit comprising one or more cut flowers and a composition for extending the vase life of cut flowers, comprising: a polynucleotide molecule that comprises at least 18 contiguous nucleotides that are essentially identical or essentially complementary to a EIN2 gene or transcript of said gene, wherein said polynucleotide comprises sense ssDNA, sense ssRNA, dsRNA, dsDNA, a double stranded DNA/RNA hybrid, anti-sense ssDNA, or anti-sense ssRNA.

19. The kit of claim 18, wherein the composition for extending the vase life of cut flowers further comprises a transfer agent, wherein the transfer agent comprises an organosilicone preparation.

20. The kit of claim 18 or 19, wherein said polynucleotide is selected from the group consisting of SEQ ID NO: 8-39, or wherein said polynucleotide comprises at least 18 contiguous nucleotides that are essentially identical or essentially complementary to SEQ ID NO:4.

21. The kit of claim 18 or 19, wherein said polynucleotide is selected from the group consisting of SEQ ID NO: 40-45, or wherein said polynucleotide comprises at least 18 contiguous nucleotides that are essentially identical or essentially complementary to SEQ ID NO: 1 or 7.

22. The kit of any one of claims 18-21, wherein:
(a) the cut flower is a carnation, the gene or the transcript is a carnation EIN2 gene or transcript, and said polynucleotide molecule is selected from the group consisting SEQ ID NO: 8-39 or said polynucleotide comprises at least 18 contiguous nucleotides that are essentially identical or essentially complementary to SEQ ID NO: 4; or
(b) the cut flower is a rose, the gene or the transcript is a rose EIN2 gene or transcript, and said polynucleotide molecule is selected from the group consisting SEQ ID NO: 40-45 or said polynucleotide comprises at least 18 contiguous nucleotides that are essentially identical or essentially complementary to SEQ ID NOs: 1 or 7.

23. The kit of any one of claims 18-22, wherein the composition for extending the vase life of cut flowers further comprises any combination of two or more polynucleotide molecules.

24. The kit of any one of claims 18-23, wherein the composition for extending the vase life of cut flowers further comprises a transfer agent, nutrients, water uptake stimulants, and chemical preservatives.


SEQUENCE LISTING (111 sequences)

SEQ ID NO: 1 - DNA, 3936 bp, Rosa hybrida var. osiana
  misc_feature (3642)..(3746): n is a, c, g, or t
atgtccaagagtctgatagctcatatggaatctgctagctctaacgctaacaatatgccaggcgttgtccatcagttgcttcctgttgttggacccatgcttctgattgcagttggatatcttgaccctggaaagtgggcggcaactgttgaagcaggttcccgttatggaactgatctggctgcagtaatgcttatattcaatttggctgctattttatgtcactatctgtcagcacggattgctgtagtcactggaagagatcttgctcaggtttgcagtgaggaatatgacaaggctacatgcatattcttaggagtacaaacagagatgtcggtgattgtgttggaccttaccatgatcctcggtatcgcacatgcacttaatcttctgtttgggtgggacttgttcacatgtgtgtttttgactgctgctaatgctgttttataccctcttttttccaccctcctggagacttgcaaggcgaaattcctttgcgtatgcatatacattgcaggatttctactgctttcctttgttcttggagtatttatcagtcaaccacaagtgccactttccatgactgggatgttaacaaaactgagtggggaaagtgctttttcactgatgagtcttcttggagcaagtataatgccccacagtttttatcttcattcttctattgtgcagcagcatcagcaacaaccaactgtttctaaggatgccttgtgtcagaaccattttgttgccatcttctgcacctttagtggtatttatctggtgaattatgccctcatgaccttagcagcaaatgtattctacacttcacgtggtttgctcacatttcaggatgcaatgtccctaatggaacaggtcttttggggtccaatagtacctgttgccttcttgctagttctctttttatcaaatcaaatcacaacattaagctggagtctggggggacaagtagtcttgaatgattttttaaaactagaccttcctggttggcttcactgtgctacaatcagaatcattgccattgttcctgctctgtattttgtttggagctcaggagctgaggggatgtaccaactgcttatatctacacaggtattggcagccttgctactgccgtcttctgtgatccctctttttcgtgttgctgcttcaagacaaataatgggagcccataagatctctcagttcgttgaattcttggccctcattacacttattgggatgcttggattaaaacttgtctttgtagtggaaatgatttttgggaatagtgattgggtggataatttgagatgggatgctgggagtagtatgtctgcacttctcatcactgcttctgcatcattttgtttgatgatttggctggcagctacaccgttaaaatctgcaagtgctcgattagagaaccaggtgtggaactgggatatgcctaagggtgtatctgagccatttagaaatagggaggagattgatatagctgaacctaattatcatagagatgcaagtgttcagaagcatgaaccatcaccatcttctgggaaggctttggatagagactcggatacagcggttgcaaattttgattttgttctgcccgaaactctcttggagcctgatcaggagcttcaatcaactacctcagaggaaaatagttctcttaatacctttcctcgctctgccaaatgcaataaggaggaaaccacatctgtggtggagtcaattcgcctcccaactgtggctagtgaggtttctgatgttacatcactgggcaccagtactgtgaaagttggatcaacagagccagttgagaagattcttggagttgaaggagacttaccaactgaaaaagatgatgatgagggagatacctgggaacctgaagattccttgaaagaggtttctgggggcaccacttcattgacatctgaaggtccaggatcattcaggagtctcagtggaaaaggtgatgaaggggggagtggtgctggaagcctttcaagattagcagggttggggcgtgctgctagacgtcaactggctgcagtactcgatgagttttggggacaactgtatgatttccatggtaatgtaattcaagaagcaaaggctaggaaactggatctgttgttggggtcagattcaaaggcttcttcctccgcttcctccgtgctgaaagatgataccactgcaaaggaagtttctggatactttccatcagtaggaggcagaggatctgatcctttaatcaactcaagtttatatgactccataaatcagccaaggctgcaaaacagtttagagtcatcatatggtgctcaaaggggatcttcctcattatggtctggccacatacagttgctggatgcgtatggacaaaattcaacctgcagtattattgactcgggtgagaggcgctattcaagtgtgcgcagtataccatcttctgagagctgggattaccagccagccacagtacacggttatcagatttcatcgtatcttaatagtaatgacagaagttctggtaacttgaatggtcaaatggaatcaccagccctaaattctgcttcttcattgggtgctggaaactacagagagtcacttgcatttaccatggggcagaagttgcaaaatgggttaggctctggacaggcctccagttttcagaaccttacggtatctcgacatagtccgttgcaatctgaaagaccatattatgatgtacactcttctggaatttctgagaatgtggtaaattcagccaatgcaaagaaataccacagtttacctgacattcaccgcgatctctacatgtctaataaaagtgctcaacgggatgctccccctgggtttgggaaaactaattatgaatcatccttgtatccaaaatctgttgcacggacaggaggtcctttggcatttgatgaactctctccatcaaaagtctatagagatgttctatcatcccaacagaattctaattttggcactggatctctctggtctagacagccttttgagcaatttggtgtagctgataataatcgttctattgggactgcagttggtagtagagcaggttctgcaggtcaggaagctatgtcagttgcagattcagaggcgaagcttcttcagtcttttagacactgcattgtgaagcttttgaagttggaagggtctgactggttgtttagacagaatgatggagtggatgaggatctaatagatcgtgtggctgccagggagaaaattatttatgaagctgaaactagagagattaatcgaacagttcacatgggtgaatctcagtacacttctgacaggaagtcaacttctgcaataaaaatgagtgatgcaaatctcacccatctcatggtttcctcagttcctaattgtggggaaggttgtatctggagatctgatctaataataagctttggggtctggtgtatccatcggattcttgatttgtcccttatggaaagccggccagagctctgggggaaatatacttacgttctcaatcgacttcaggggattattgattctgcgttttcgaagcctcgttctccaatgtctccatgcttctgccttcaaattgcagcagcacagcagcagaagtcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgcaatctcttgtcgaaagggccgaacggggacggcagccggtgatgtggctttcccgaagggaaaagaaaatctggcatctgtcctcaaacgctacaagcggcgattatccaacaaacctgtttgcactcaggaggggccttcccgttcacggaagggtgctccaacatctgctccttatgggtcataa

SEQ ID NO: 2 - DNA, 3885 bp, Arabidopsis thaliana
atggaagctgaaattgtgaatgtgagacctcagctagggtttatccagagaatggttcctgctctacttcctgtccttttggtttctgtcggatatattgatcccgggaaatgggttgcaaatatcgaaggaggtgctcgtttcgggtatgacttggtggcaattactctgcttttcaattttgccgccatcttatgccaatatgttgcagctcgcataagcgttgtgactggtaaacacttggctcagatctgcaatgaagaatatgacaagtggacgtgcatgttcttgggcattcaggcggagttctcagcaattctgctcgaccttaccatggttgtgggagttgcgcatgcacttaaccttttgtttggggtggagttatccactggagtgtttttggccgccatggatgcgtttttatttcctgttttcgcctctttccttgaaaatggtatggcaaatacagtatccatttactctgcaggcctggtattacttctctatgtatctggcgtcttgctgagtcagtctgagatcccactctctatgaatggagtgttaactcggttaaatggagagagcgcattcgcactgatgggtcttcttggcgcaagcatcgtccctcacaatttttatatccattcttattttgctggggaaagtacatcttcgtctgatgtcgacaagagcagcttgtgtcaagaccatttgttcgccatctttggtgtcttcagcggactgtcacttgtaaattatgtattgatgaatgcagcagctaatgtgtttcacagtactggccttgtggtactgacttttcacgatgccttgtcactaatggagcaggtatttatgagtccgctcattccagtggtctttttgatgctcttgttcttctctagtcaaattaccgcactagcttgggctttcggtggagaggtcgtcctgcatgacttcctgaagatagaaatacccgcttggcttcatcgtgctacaatcagaattcttgcagttgctcctgcgctttattgtgtatggacatctggtgcagacggaatataccagttacttatattcacccaggtcttggtggcaatgatgcttccttgctcggtaataccgcttttccgcattgcttcgtcgagacaaatcatgggtgtccataaaatccctcaggttggcgagttcctcgcacttacaacgtttttgggatttctggggttgaatgttgtttttgttgttgagatggtatttgggagcagtgactgggctggtggtttgagatggaataccgtgatgggcacctcgattcagtacaccactctgcttgtatcgtcatgtgcatccttatgcctgatactctggctggcagccacgccgctgaaatctgcgagtaacagagcggaagctcaaatatggaacatggatgctcaaaatgctttatcttatccatctgttcaagaagaggaaattgaaagaacagaaacaaggaggaacgaagacgaatcaatagtgcggttggaaagcagggtaaaggatcagttggatactacgtctgttactagctcggtctatgatttgccagagaacattctaatgacggatcaagaaatccgttcgagccctccagaggaaagagagttggatgtaaagtactctacctctcaagttagtagtcttaaggaagactctgatgtaaaggaacagtctgtattgcagtcaacagtggttaatgaggtcagtgataaggatctgattgttgaaacaaagatggcgaaaattgaaccaatgagtcctgtggagaagattgttagcatggagaataacagcaagtttattgaaaaggatgttgaaggggtttcatgggaaacagaagaagctaccaaagctgctcctacaagcaactttactgtcggatctgatggtcctccttcattccgcagcttaagtggggaagggggaagtgggactggaagcctttcacggttgcaaggtttgggacgtgctgcccggagacacttatctgcgatccttgatgaattttggggacatttatatgattttcatgggcaattggttgctgaagccagggcaaagaaactagatcagctgtttggcactgatcaaaagtcagcctcttctatgaaagcagattcgtttggaaaagacattagcagtggatattgcatgtcaccaactgcgaagggaatggattcacagatgacttcaagtttatatgattcactgaagcagcagaggacaccgggaagtatcgattcgttgtatggattacaaagaggttcgtcaccgtcaccgttggtcaaccgtatgcagatgttgggtgcatatggtaacaccactaataataataatgcttacgaattgagtgagagaagatactctagcctgcgtgctccatcatcttcagagggttgggaacaccaacaaccagctacagttcacggataccagatgaagtcatatgtagacaatttggcaaaagaaaggcttgaagccttacaatcccgtggagagatcccgacatcgagatctatggcgcttggtacattgagctatacacagcaacttgctttagccttgaaacagaagtcccagaatggtctaacccctggaccagctcctgggtttgagaattttgctgggtctagaagcatatcgcgacaatctgaaagatcttattacggtgttccatcttctggcaatactgatactgttggcgcagcagtagccaatgagaaaaaatatagtagcatgccagatatctcaggattgtctatgtccgcaaggaacatgcatttaccaaacaacaagagtggatactgggatccgtcaagtggaggaggagggtatggtgcgtcttatggtcggttaagcaatgaatcatcgttatattctaatttggggtcacgggtgggagtaccctcgacttatgatgacatttctcaatcaagaggaggctacagagatgcctacagtttgccacagagtgcaacaacagggaccggatcgctttggtccagacagccctttgagcagtttggtgtagcggagaggaatggtgctgttggtgaggagctcaggaatagatcgaatccgatcaatatagacaacaacgcttcttctaatgttgatgcagaggctaagcttcttcagtcgttcaggcactgtattctaaagcttattaaacttgaaggatccgagtggttgtttggacaaagcgatggagttgatgaagaactgattgaccgggtagctgcacgagagaagtttatctatgaagctgaagctcgagaaataaaccaggtgggtcacatgggggagccactaatttcatcggttcctaactgtggagatggttgcgtttggagagctgatttgattgtgagctttggagtttggtgcattcaccgtgtccttgacttgtctctcatggagagtcggcctgagctttggggaaagtacacttacgttctcaaccgcctacagggagtgattgatccggcgttctcaaagctgcggacaccaatgacaccgtgcttttgccttcagattccagcgagccaccagagagcgagtccgacttcagctaacggaatgttacctccggctgcaaaaccggctaaaggcaaatgcacaaccgcagtcacacttcttgatctaatcaaagacgttgaaatggcaatctcttgtagaaaaggccgaaccggtacagctgcaggtgatgtggctttcccaaaggggaaagagaatttggcttcggttttgaagcggtataaacgtcggttatcgaataaaccagtaggtatgaatcaggatggacccggttcaagaaaaaacgtgactgcgtacggatcattgggttga

SEQ ID NO: 3 - DNA, 3951 bp, Solanum lycopersicum
atggagtctgaaactctgactagagaatataggcggcccagcatgcttcagcgagtactttctgcttctgtgccaatgctgttgattgcagttggctatgttgatcctgggaaatgggctgcaatggttgatggaggagcccgatttgggtttgatttggtcatgctagtactcttgttcaattttgctgccattctgtgccagtatctgtctgcttgtatagccttggttacagaccgagatcttgcgcagatttgcagtgaagaatatgacaaagttacatgcatattcctaggaattcaagctgaggtttcgatgattgctttggacctcacaatggttttgggcactgcccatgggcttaatgttgtgtttggagttgacctgtttagctgtgttttcctgactgcaaccggtgccattttgtttccactgcttgcttctctcttggacaatggcagtgcaaaattcttatgtattggctgggcaagctctgtactgctctcttatgtttttggagtggttataactctacctgaaactccattctccattggtggtgtgctgaataagtttagtggagagagtgcatttgcattgatgagtcctcttggagcaagtattatgcctcacaatttttacctccattcttctattgtacagcaaggtaaggaatcaacagagctttccaggggagctctgtgtcaggaccatttttttgccattgttttcatattcagtggcattttcctggtcaactatgccgcgatgaattcagcagcgaatgtgtcttacagtactggccttttgttgctgacatttcaggacacattgtcattgctcgatcaggttttcagaagctcagttgcaccattcaccataatgctggttacatttatttccaatcaagttacaccactaacttgggatcttggtagacaagcagttgtgcatgacttatttggaatggacatcccaggctggcttcatcatgtgacgatcagagttatttccattgtcccagctctttattgtgtatggagttcaggagctgaaggcctatatcagttacttatactgacacaggttgtggtggctcttgtccttccatcttctgtcatacccctgttcagagttgcttcttccagatcaattatgggtatccacaaaatttctcagttaatggagttcttatctcttggcacatttattggcttacttggcctaaagattatatttgtcatagagatgatatttggaaatagtgattgggttaataatttgaagtggaatattgggagtagtgtgtctactccatatttttttctcctcatcgcagcctctttatgtctttgtctgatgctgtggttagcagttactcctctgaaatctgcaagttccaggttcgatgctcaggcgtttctgcaaacgcatgtgcctgagccatattcggagtgtaatcaacttggtgcgagtaatgctatgtttggtctagtagaaggatcctcccaaaagcaagaaggtgcatttcatgtggaaaaatccttggtaagccatccagatttatcaactaaagatcctgatcaactcttgccagaatctctcttggattttgaaaaggtccatcagttggctactattgatgagagcaaatctgaaacaacattttcagctcctgctgtcgttcatcctgaggtacctgtatcagcaggagcaagtcccagtgtgaaaagtgtttgtaatgaggtttctggtgttgtatcagtggataccagtgtcttcaatactgaaactgtggatgtcgcagagaagactctcagaattgaaggggacatggcaaatgacagggatgatggagattcgtgggaagagcctgaagaggcaatcaaaggagtatctgagaacgctcaatcttttatttctgatggtccggggtcatacaaaagtctaagtggaaaactagaggacacggggagtggaacaggaagtctatcaagattagcaggtcttggtcgtgcagctaggaggcagttaacagaagctctaaatgagttttgggggcagctttttgattaccatggcgtggcaacagcagaagcgaagtccaagaaactggatataatacttggtctggattcaaagatgaatccaaaacctgcccctgcatcattaaaagttgaaagcagtgcgtatattccatcggggagtgcaaggataccagagcctctgatcaactcgcatgtgtactctcccaagcagcaatttgcgtcaaacattgtggactctgcttatagagtcccaaaggagccatcttcgacatcttctatgtggtctaaccatatgaaattagtaggtgcatatgtgcaaagttccaacagcaacatgcttgactcaggggagaggcgctattctagtatgcggattccagcgacttctgctggctatgatcagcagcctgccactgtgcatggatatcagattactgcttaccttaatcaacttgcgaaagaaagaggatctgattatttaaatgggcaactggagtcaccatctcctcgttctgtatcatcactgacgtcaaactatgcagaaccattggctcgtgtttcggggcaaaaacctcagagtggagtcagtagtcgagcaccacctggttttggaaatgtccctgtaggccgaaataattcgatgcagcccactaacactacttctgtcgaccatagctctactgaaactgctgaaagcgtggctggttcagccaactctaagaagtactacagcttgcctgatatctcagggcgctatgttcctcgccaagattctatagtgtcagatgcgagagctcaatggtacaattccatgggattcggacaatctggtggtcgatctacatacgaacaagcctatatgagtggttcactaagggcaggtggtcctcagaggtatgaacattctcctaaagtctgcagagatgcattctccttgcagtacagctccaattcagggactggatccctgtggtctagacagccttttgagcaatttggtgtagctggtaagccagatgttggtagcggcgatcatggaactgtgctgagttcctctgctcaagagagtacatctacggttgacttggaagctaagctgcttcagtctttcagaagttgtattgtgaaacttttgaaactggaaggatctgagtggttatttaggcaagatgatggggctgatgaggatcttataggtcggattgctgcaagagagaaatttctctatgaagctgaaactagggagataagtagattgaccaacattggtgaatcacacttctcttccaacaggaaacccggttctgccccaaaacctgaagagatggattacaccaagttcttggtgatgtcagttccccactgcggagaaggttgtgtttggaaagtagatctgattataagcttcggtgtgtggtgcattcacagaattcttgagctttcacttatggaaagtaggccagagttgtggggcaaatatacctatgttctcaaccgtcttcagggcatagtagatctggcattttcaaagccccattctccgacgagccattgtttttgtcttcaaattccggctggccgccagcaaaaggcaagcccccctccaatttctaatggaaacttgccgccacaagcaaaacagggtcgaggaaaatgcacgactgcagcaatgctcttagagatgatcaaagacgtggagacagcaatttcctgtcgaaagggacgaacgggcactgcagcaggggatgtagcctttcctaaaggaaaagagaacctggcatccgtcctcaagcgctataaacgtcgattatccaataagccggtaggaaaccaggaggtggctggagtcgccggaccgcgcaaagtaacgctgtctgcctcatcaccccctttcgtcttgtaa

SEQ ID NO: 4 - DNA, 3828 bp, Dianthus caryophyllus
atggcggaggttttgttgccggctgttactccggtggtgttgattttgatcggatatattgaccccggaaagtgggctacttacgtcgatgttggtgctcgttatggcggtgatcttgtcgtgtttgcgctcttgtttaacgtcgttggtgtcttgtgtcattatctttctgctcgtgttactatcatcactggacgcaatttgacgcagatctgttcccaggagtatgacagactgacctgtttttttctcgggcttcaagcagaactttctgtgatcaccttagatcttactatgattattggtatcgcccatggactcaacatgatcttcggtctgaatttgttcgttggtattctgctgacagcactgaatgcgttgctgtttccatttttttcctccctcctggaaagctccaaggcaaaattcgttgtcgtatgcttggctggattaacaatcgctagctatgtgcttggagctctgtcaagtctaccggaatttacgacttcttcaaatttggtagctaagtttagtggagagagtgcttttgccttgatgggtcttcttggatcaaatgtcatgcctcacaatttttatctccattcttccattgtacagtggtatcaagggcaaactagtgtatcgacgagtgcgtggtctcaagataattttattctcaacttcgccatttccggtggcatattttcggcaacttttgtacttatgaattcggtcgccaatggtgtatatagtacaggtgttggtttgctatctatccaggatgcattatctctgcttgatcagacatacaggaattccataatacccatcggtgctttcgtggttttatttttggccaatcaaatcgcgtcgttaagctgggaatttcatggtgagggggccaaaagtggggaaaaaatgatgcacgactttttcgacatggatcttcctgtgtggattcatcgtgctgctgttagaatttttgctgctgtgattgcactgttttgtttgtggcactctggagccgaaggaatgttccatttgctgatatgcacacaagtaatcgtggctcttctgcttccgtcttctgtgataccgcttttccgaattgcatcttgccgacctattatggatctgcgcaagatgtctcccgcacttgagttcatagccatcctaacgttcatgggaatgctttgtttggagcttatttttgtggtggaattaatttttggggagagtgaatgggtcgttaacctgcgttggacaattagcaatggtgcatctatgtcgtatattctgcttctcgttgccgtctgtgtttcactttttttcatgttttgggttgcagcgacccctctaaaatcctcgattagcaaactgaattctcagccttggaatttgaatgctcagcaagtttctcctggatcaagtattgaaagggagaataatgatattaccgagacaatatactccaaagaggaatctattaatgtcgagaaagaagttataacattggaggagagttccttactgaaccattccgacacaccagatgctaactgtgatatcaatttgccggacacaattatggacacagttcaggagctttacgtggctaatagcgacgaattgcccggtaactcatctgcatgccatcctaagccaaagcagttagcaacttcttcggaatcagtggcagtctctagcgtgtcaaccaggattgaagacgatacttttcagaaatcaagtaacgcggtaaacaatcggatggatgctgatgaaaagaccttgagagttgagggtgactcgccacctgagaaacaagacgacagaaatgcgtgggaacctggggaatcatctaaaggcatttccgaagttgatccatctaccgcctctgatggtccaggatcattcagaagtctttctggtggcggcagtctttctagactttcaggactcggccgtgcagcaagacgacagatggcttcggtccttgatgaattttggggacaactctatgatttccatggtcaaataactcaagaagcaaggagtaagaaactggatttgcttcttggtgcagattctaagccatcatcacaatcggtatcgaaatcaaatcctgcggggagggagttggtaatgcagtcacaatctttgggaggcagagtttctggcaatacgattaactcaagtttatataacagtccagatcagcagaagttgttcgacagtatagaagcggcgtataaggctcatagagcatctacttcgatatggtcaaacccgccaccagtttcagacacgtatgtgcagaactctaaccgcagtcttcttgattcaggggagaagcggtatcatagtgtacgtctcccgtcatcttctgagaggtcagaatatcaggcagcaactgtacatggttatcaattggcatcttatgctaatcgtgccgccaaagacaggtctgattatgcatttggccgactagaatctgtgcctcaaaagtcaccgtctttggttcccaacaattacgaagaatcttttggttttacgtcggggcggaattctgaaaatggacttcatgctgcacagacttcgagctttcaaaactttccagtgcaaagaagaaattttgaccaatttgatagagctagttacgaattttctgctggaccaattgaaaggatgagtaaccataataatgccaaacaatatcacagttcgccagacatttctgcactttctgcacgactccgaaattcctatttgtcaaatgggaatatgcagttcgacagccccaacacttcttcgggatttcgagcaacagttggtcggacaacttatgagccatccccaatacgcagtaccggcggctctactgggtcaagacctgtgggaccccttgcgtttgatgaactttctccttctatggcatattgtgacgctatttcgctcagctcgagctcgggtacacgctcgctttgggctagacagccttatgaacagtttgggttggctaacaacactagcaatctcggagctcttgctgctggaaataggtgcacaacgacagctcgagagcctccgtttgccgagattgagagcaaattgcttcagtcgttgaggcactgcattttgaagttgctgaaattggaaggctccgaatggctgttcagggaaaatgacggtgttgacgaagatctcattgatcgagtggttactcgagagagatttatttttgaggtagagtctcgagaatttaaacaagcgtctccgttaggtagtagtgacgaggcagctaacgcacatctgatctcttcagttcctcactgtggagagggttgcgtttggaagttagacctcattgccagtttcggtgtgtggtgcattcatcgtatactcgagctctctctcatggaaagtcggccagaactgtggggaaagtacacttacgtgctaaatcgtcttcagggtgtaattgatttggcgtttttcaaacctcggacgtcaatgtccccctgcttctgcctccaagtaccggcatcataccagaggaaatcaacgtctccgttttcaaacgacaagttgcctccagctataagaccagctaagggaaaagtaacaaccgcctcaacgattctcgaagtaatcaaagatgtcgaaatcgcaatctcgtgtcgcaaaggtcggtctggaactgctgcaggtgacgtcgcgtttcccaagggaaaagagaatctggcgtctgtcctgaaacgctataaacgccgcttatctaacagagcagccggggcaaatgacaatggccagggattacgaaagctctaa

SEQ ID NO: 5 - DNA, 3915 bp, Prunus persica
atggctaatttggaatctgctaatcctagtgctaacaatatgttgggcgttctacatcggttgcttcctgttgttggaccggcacttctgatttcagttggacatcttgaccctggaaagtgggcggcaactgctgaagcaggtgcccgttttggctctgacctggcagcattgatgcttattttcaattttgcagctattttatgtcactatctgtcagctcggattggtgtagtcactggaagagatcttgcgcagatatgcagtgaggagtatgacaaaggcacatgcatattcttaggagttcaaacagaggtttctgtgattctgtcagaccttaccatgatcctcggtatcgcacatggacttaatcttctgtttgggtgggacttgttcacttgcgtgtttttgactgctgttaatgctgttttgtaccctcttttttccaccctcctggagacttgcaaggcgaaggtcctatgcgtatgcattgcgggatttatacagctttccttcgttcttggagtaattatcagtcaaccagaaatgtcattttccatgaatgggatgctaacaaagctgagtggggagagtgcctttgcattgatgagtcttcttggagcaagtataatgcctcacagtctctatcttcattcttcgattgtgcagcagtatcagtgtcagccaactgtttccagggatgctttgtgtcaccaccatttagttgccatcttgtgcatctttagtggtatttatctggtgaattatgcccttatgacctcagcggagaatgaatactcgggccttggtttgcttacatttcaggatgtaatgtcactaatcggacaggtcttttggggtccaatagtatctggtgcctacctgctggttctctttgtttcaaatcaaatcacaacattaagctggagtctgggtggacaagtagttttgaatgattttttgaaactagaccttcctggttggcttcattgtgccacaatcagaattattgctattgttccagctctttatttcgtttggagttcaggagctgaagggatgtatcaacttcttatattcacacaagtgttggcagctctgctactgccatcttctgtgatccctctttttcgaattgctgcttcaagaccaataatgggtgtccataaagtttctcaatttgttgaatttttatccctgattacactaattgggatgcttggattgaaaatcatatttgtagtagaagtgattgttgggaatagtgattgggttaataatttgaggtcgaatgccgggagtagtatgtctgttccttgtgtacttctactcactgcttgtgcaacattttgtttgatgatttggctggcagctaccccattaaaatctgcaagtgcacgattagaggctcaggtgtggatctgggatatgcatatgggttcacctgattcaataacaaagaaagaggagatcaatatttctgaacctaaatatcatagagaggtaagtgtccagaagcatgaaccatcaccatcatttgggagggctttggatagtgattcagaagtagcgagttttgatcttgatctgcctgaaactatcacagagcctgatgaggagcatcacctaactactgtagcggaaaatggttctcgtattacctttcctcattcccctaaatgccatatggagggatccacatctacagtggagtcaactccagtctcaactgtggttaatgaggtttctgatgttacattggagggcaccagtgcattgaagatcgaatcaacagagccaattgaaaagactgttggagttgaaggagttgaaggagacttaccaaatgaaaaagatgatgatgagggagatacctgggagcctgaagattcattgaaaggggtttctgagagcactgctccattgacatctgagggtccaggatcatttaggagtctaagtggaaaaggtgacgaaggggggagtagtgctggtagcctttcaagattagcagggttggggcgtgctgcgagacgtcaactagctgcagtacttgacgaattttggggacagctgtatgatttccatgggaatgtaattcaagaagcaaaggctaagaaactggatcttttgttggggttagattcaaaggctgcgtcctcctcattgaaagttgatactagtgcaaaggagctttccggatattttccatctgcaggaggcagaggatctgatcctattatgaactcaagtttatatgactctccgaagcagcagagggttcaaagcagtttagagtcatatggggtccaaaggggatcttccgcgttgttgcccagccgtgtgcagttgttggatgcctatgtgcaaaattcaagccgcagcgttattgactctggtgagaggcgctattcaagtgtgcgcagtctcccgtcttctgagagttgggactaccagccagccacaatacatagttatcatccctcatatctcaatcgaattgcaaaggacagaggttttgataatttgaacggtcaaatggagtcagcagccctacaatccgcttcttcattgggtgctgcaaactacagagattcacttgcatttaccatggggcagaagttacaaaatgggttaggctctggtcaggcctccattttccaaaatcatacagtatctagaaatagtccgttgcaatctgaaagaccgtattatgatctgcacccttctggaattgctgagaatgtggtaagttcagcaaatgcaaagaaataccatagtttacccgacattcaccgggatctttacatgccggagaaaagtgccaactgggaaagtcctgtggggtatggatcatctactgggataacaaattatgaatcatccttgtattcaaattctggagcacgaacaggagctcctttggcattcgatcaactctctccatcacaagtctacagagatgctttttcatcacagcaaaattctagttttaacactggatccctctggtctagacagccttttgagcaatttggtgtagctgataataatcgtactattgggagtggaggatttggttatcgggcaggttctgtaagtcaagaagctacttcagttgcagattcagaggccaagcttcttcagtctttcaggcattgcattgtgaaacttttgaaattggaaggatctgactggttgtttacgcagaatgatggggttgatgaggatctaattgatcgcgtggctgcaagggagaaatttctttatgaagctgaaactagagagatgaatcgaacagttcacatgggtgaacctcaataccatccttctgataggaagtctgtttctgcattgaagaataatgatgcaaattgcacctcttttatggttcctacttgtggggagggttgtatttggagatcagatttgatagtaagcttcggggtctggtgtatccatcgtattcttgatttgtcactcatggaaagccggccagagctatgggggaaatatacctacgtcctcaaccgacttcaggggattattgattcagcattttcaaagcctcgcactccaatgtcgccatgcttctgccttcaaatttctgcagtacaccagctgaagtcaagtccatctttttcaaatggaataccccctgctgcaaaaccagccaggggaaaatgcacaacggcagtaacgcttctagacataatcaaggatgtggagattgcaatatcttgtcgtaagggccgaacggggacagcagctggcgatgtagctttcccgaagggaaaagaaaatctggcgtctgtactcaaacgctacaagcgtcgattaaccaacaaaactgctggcgctcacgagggtcctggttcacgcaaggttcagacatctgctccttatgggtcatag

SEQ ID NO: 6 - DNA, 3933 bp, Petunia hybrida
atggaatctgaaactcagactatagcttataggcagcccagcatgcttcaacgaatactttctgcttctatgcctatgctactgattgcaattggctatgttgatcctggaaaatgggctgcaatggttgatggaggagcccgttttggatttgatttgatcatgctagcacttctattcaattttgctgccattctgtgccagtatctctcagcttgtatagccttggttacagaccaagatcttgcccagatttgcagtgaagaatatggcaaagttacatgcatattcctaggaattcaagctgaggtttcgatgattgccttggacctcacaatggttttgggtactgcacatgggcttaatgttgtgtttggagttgacctttttagctgtgtttttctggctgcaactggtgccattttgtttccactgcttgcatctctcttggacaatggcagtgcaaaattcatatgcattggctgggcaagctctatactgctctcttatgtttttggagtggtcataagtcaacctgaaagtccattctccattggtgggatgctgaataagttcagtggagagagtgcatttgcattgatgagtcttcttggagcaagtattatgcctcacaatttttaccttcattcttctattgtacagcaaggtaaggaatcaacaaacctttccaggggagccctgtgtcaggaccatttttttgccattgttttcgtattcagtggcattttcctggtcaactatgccataatgaattcagcagctaacgtgtctttcagcactggccttttattgcttacatttcaggactcattgtcattgctcgatcaggtgttcagaagttcagtggcaccattcagcataatgctagttacgtttatttccaatcaaattacgccactaacttgggatcttggtagacaagcagttgtgcacgacttattcggaatggacattccgggctggcttcatcatgtgacaatcagagttatttccgttgttccagccctttattgcgtatggaactcaggagctgaaggactatatcagctactaatagttacacaggttgtggttgctcttgtgcttccgtcttctgtcatacccctgttcagagttgcttcttcccggtcaataatgggtatccataaaatttctcaattaatggagttcttatctcttggcacatttatcggcttactcggcttaaagattatatttgtcatagagatgatatttggaaatagtgattgggttaataatttgaagtggagtatcgggagtggcgtatctactccatatgtttttctactcattgcagcctctttatctctttgtctgatgctgtggttagcagttactccgctgaaatctgcaagttccaggttcgatgctcaggcatttctccaaacacctatgccagagtcatatcgagagcataatcaagttgatgtgagtgatactacctttggtctagaaaggtccacccaaaagcaagaacctgcatttcatgtggaaaaatccttgggaagccatcctgatttgtcaacttcagaccctgatgaaatcttgcccgaatcactcttggattttgagaaggtccatcatttgactaccattgatgagagcaaatctgaaactacattttcaaccccttctttcagctgtcctgaggtatctgcatcagcaggagaaactgcgaaaagtgttctcaatgaggtgtctggtggtgaatctgtggataccagggatttcaatgctgcatctgtggatgtagtagagaagacactcagaattgaaggggacacgccaaccgacaaggatgacgatggagattcatgggagcctgatgacgtacctaaagatgtatctgagaacacccaatcttatacttctgatggtccggaatcattcaagagtcttagtgtcaggtcagaagacacagggagtggtacaggaagtctatcaagattagcaggtcttggtcgtgcagctaggaggcagttaacagtagttctagatgagttttggggacagctttttgattaccatgggatgcccacatcacaagcaaagttcaagaaactggatgtaatactcggtctggatacaaaagtggatccaaaacctgccccagtgtcattaaaactggagaacagcaggggtgattctaatgcgtatattccatctggtagtgcaagggtacctgagtcatggatcaactcgaatatatactctcccaagcagcaatgtgcatcaggtgctctggactctggttatagagtcccgaaggagccagcttcatggtctagccatatgaaattattagatgcatatgtgcaaagttccagcggcaacacacttgactcgggtgagaggcgctattccagcatgcggattcctgcgtcttctgctggctatgatcagcagcctgcgactgtgcatggatatcagatctccgcttacctaagtcaaattgctaaaggaagaggatctgattatttaaatgggcaactggagtcagcatcccctcgttctgtatcatcattgacgtcaaaccatgctgaaccattagctcgtgctttggggcaaaaacctcagagtggagtgagtagtcgagcaccacctggttttggaagtgtccctgcccgaaataactcgatgcagcccgttaacacttctactgaccttagctctacggaaaatgctgagagcgtagctggctcagccaactcgaagaagtattacagcttgcctgatatatcaggacgctatgttcctcgccaagattcttcactcccagatgggagagctcaatggtacaattccatgggatatggacaatctattggccgatctgcgtacgaacaaccctatatgactggtccaatgagggctggtggtcctccaaggtttgaacattctccttctaaagtctgcagagatgccttcaccttgcagtacagttccaattcggggactggatccctgtggtctagacagccttttgagcaatttggtgtagctggtaaggctgatgttagcagtgatcatggaactgtgcagagttcatctactcaggagagcacatctttggttgatttggaagctaagctgcttcagtctttcagaagttgtattgtgaaacttttgaaactggaaggatctgagtggttatttaggcaagatgatggtgctgatgaggaccttatagatcggattgctgcaagagaaaaatttctctatgaagctgaaaccagggagataagcagattgaccaatattggtgaatcacagttctcttctaacaggaaacctggttctgcccaaaaaccagaagagatggattacaccaagttcttagtgatgtcagttcctcactgtggggaaggctgtgtttggaaagtagatctggttgtaagcttcggtgtatggtgcattcacagaattcttgagctttcactcatggaaagtcggccagagctgtggggtaaatatacctattgtctcaatcgtcttcagggcatagtagatctggcattttccaaaccccgttctccaacaagtcattgtttttgtcttcaaattccaattggccggcagcaaaagtcaagccccactcccatttcaaatggaagtttgccaccacaagcaaaacagggccgaggaaaatgcacaactgcaccgatgctcttagatatgatcaaagacgtggagatggcaatctcttgtcgaaagggacgaacaggcactgcagcaggggacgtggcttttcctaaagggaaagaaaacttagcatctgtcctcaaacgctataaacgtcgactatcaaataagccagtagggaaccaggaggctggtggaggtccacaacgcaaagtaacgtcaccctcgtccacatcttttggcttgtaa

SEQ ID NO: 7 - DNA, 3936 bp, Rosa hybrida var. freedom
  misc_feature (2445)..(2462): n is a, c, g, or t
atgtccaagagtctgatagctcatatggaatctgctagctctaacgctaacaatatgccaggcgttgtccatcagttgcttcctgttgttggacccatgcttctgattgcagttggatatcttgaccctggaaagtgggcggcaactgttgaagcaggttcccgttatggaactgatctggctgcagtaatgcttatattcaatttggctgctattttatgtcactatctgtcagcacggattgctgtagtcactggaagagatcttgctcaggtttgcagtgaggaatatgacaaggctacatgcatattcttaggagtacaaacagagatgtcggtgattgtgttggaccttaccatgatcctcggtatcgcacatgcacttaatcttctgtttgggtgggacttgttcacatgtgtgtttttgactgctgctaatgctgttttataccctcttttttccaccctcctggagacttgcaaggcgaaattcctttgcgtatgcatatacattgcaggatttctactgctttcctttgttcttggagtatttatcagtcaaccacaagtgccactttccatgactgggatgttaacaaaactgagtggggaaagtgctttttcactgatgagtcttcttggagcaagtataatgccccacagtttttatcttcattcttctattgtgcagcagcatcagcaacaaccaactgtttctaaggatgccttgtgtcagaaccattttgttgccatcttctgcacctttagtggtatttatctggtgaattatgccctcatgaccttagcagcaaatgtattctacacttcacgtggtttgctcacatttcaggatgcaatgtccctaatggaacaggtcttttggggtccaatagtacctgttgccttcttgctagttctctttttatcaaatcaaatcacaacattaagctggagtctggggggacaagtagtcttgaatgattttttaaaactagaccttcctggttggcttcactgtgctacaatcagaatcattgccattgttcctgctctgtattttgtttggagctcaggagctgaggggatgtaccaactgcttatatctacacaggtattggcagccttgctactgccgtcttctgtgatccctctttttcgtgttgctgcttcaagacaaataatgggagcccataagatctctcagttcgttgaattcttggccctcattacacttattgggatgcttggattaaaacttgtctttgtagtggaaatgatttttgggaatagtgattgggtggataatttgagatgggatgctgggagtagtatgtctgcacttctcatcactgcttctgcatcattttgtttgatgatttggctggcagctacaccgttaaaatctgcaagtgctcgattagagaaccaggtgtggaactgggatatgcctaagggtgtatctgagccatttagaaatagggaggagattgatatagctgaacctaattatcatagagatgcaagtgttcagaagcatgaaccatcaccatcttctgggaaggctttggatagagactcggatacagcggttgcaaattttgattttgttctgcccgaaactctcttggagcctgatcaggagcttcaatcaactacctcagaggaaaatagttctcttaatacctttcctcgctctgccaaatgcaataaggaggaaaccacatctgtggtggagtcaattcgcctcccaactgtggctagtgaggtttctgatgttacatcactgggcaccagtactgtgaaagttggatcaacagagccagttgagaagattcttggagttgaaggagacttaccaactgaaaaagatgatgatgagggagatacctgggaacctgaagattccttgaaagaggtttctgggggcaccacttcattgacatctgaaggtccaggatcattcaggagtctcagtggaaaaggtgatgaaggggggagtggtgctggaagcctttcaagattagcagggttggggcgtgctgctagacgtcaactggctgcagtactcgatgagttttggggacaactgtatgatttccatggtaatgtaattcaagaagcaaaggctaggaaactggatctgttgttggggtcagattcaaaggcttcttcctccgcttcctccgtgctgaaagatgataccactgcaaaggaagtttctggatactttccatcagtaggaggcagaggatctgatcctttaatcaactcaagtttatatgactccataaatcagccaaggctgcaaaacagtttagagtcatcatatggtgctcaaaggggatcttcctcattatggtctggccacatacagttgctggatgcgtatggacaaaattcaacctgcagtattattgactcgggtgagaggcgctattcaagtgtnnnnnnnnnnnnnnnnnntgagagctgggattaccagccagccacagtacacggttatcagatttcatcgtatcttaatagtaatgacagaagttctggtaacttgaatggtcaaatggaatcaccagccctaaattctgcttcttcattgggtgctggaaactacagagagtcacttgcatttaccatggggcagaagttgcaaaatgggttaggctctggacaggcctccagttttcagaaccttacggtatctcgacatagtccgttgcaatctgaaagaccatattatgatgtacactcttctggaatttctgagaatgtggtaaattcagccaatgcaaagaaataccacagtttacctgacattcaccgcgatctctacatgtctaataaaagtgctcaacgggatgctccccctgggtttgggaaaactaattatgaatcatccttgtatccaaaatctgttgcacggacaggaggtcctttggcatttgatgaactctctccatcaaaagtctatagagatgttctatcatcccaacagaattctaattttggcactggatctctctggtctagacagccttttgagcaatttggtgtagctgataataatcgttctattgggactgcagttggtagtagagcaggttctgcaggtcaggaagctatgtcagttgcagattcagaggcgaagcttcttcagtcttttagacactgcattgtgaagcttttgaagttggaagggtctgactggttgtttagacagaatgatggagtggatgaggatctaatagatcgtgtggctgccagggagaaaattatttatgaagctgaaactagagagattaatcgaacagttcacatgggtgaatctcagtacacttctgacaggaagtcaacttctgcaataaaaatgagtgatgcaaatctcacccatctcatggtttcctcagttcctaattgtggggaaggttgtatctggagatctgatctaataataagctttggggtctggtgtatccatcggattcttgatttgtcccttatggaaagccggccagagctctgggggaaatatacttacgttctcaatcgacttcaggggattattgattctgcgttttcgaagcctcgttctccaatgtctccatgcttctgccttcaaattgcagcagcacagcagcagaagtccagtccaacattttcgaatggaatgttaccccctgctgcgaaaccagccaggggaaaatgcactacagctgcaacactcgcggacataatcaaggatgtggagactgcaatctcttgtcgaaagggccgaacggggacggcagccggtgatgtggctttcccgaagggaaaagaaaatctggcatctgtcctcaaacgctacaagcggcgattatccaacaaacctgtttgcactcaggaggggccttcccgttcacggaagggtgctccaacatctgctccttatgggtcataa

SEQ ID NO: 8 - DNA, 21 bp, Artificial
  synthetic
gccgtagcgagcatacgtatg

SEQ ID NO: 9 - DNA, 22 bp, Dianthus caryophyllus
tacgtatgctcgctaccggcgc

SEQ ID NO: 10 - DNA, 21 bp, Dianthus caryophyllus
ttctcgtgctgctgttagaat

SEQ ID NO: 11 - DNA, 22 bp, Dianthus caryophyllus
tctaacagcagcacgatgaatc

SEQ ID NO: 12 - DNA, 21 bp, Dianthus caryophyllus
cagatagaagcggcgtataag

SEQ ID NO: 13 - DNA, 22 bp, Dianthus caryophyllus
tatacgccgcttctatactgtc

SEQ ID NO: 14 - DNA, 21 bp, Dianthus caryophyllus
gaggcgtggtctcaagataat

SEQ ID NO: 15 - DNA, 22 bp, Dianthus caryophyllus
tatcttgagaccacgcactcgt

SEQ ID NO: 16 - DNA, 21 bp, Dianthus caryophyllus
agggttggtttgctatctatc

SEQ ID NO: 17 - DNA, 22 bp, Dianthus caryophyllus
tagatagcaaaccaacacctgt

SEQ ID NO: 18 - DNA, 21 bp, Dianthus caryophyllus
ggaagtgaatgggtcgttaac

SEQ ID NO: 19 - DNA, 22 bp, Dianthus caryophyllus
taacgacccattcactctcccc

SEQ ID NO: 20 - DNA, 21 bp, Dianthus caryophyllus
gaccagttcaggagctttacg

SEQ ID NO: 21 - DNA, 22 bp, Dianthus caryophyllus
taaagctcctgaactgtgtcca

SEQ ID NO: 22 - DNA, 21 bp, Dianthus caryophyllus
atcagtaacgcggtaaacaat

SEQ ID NO: 23 - DNA, 22 bp, Dianthus caryophyllus
tgtttaccgcgttacttgattt

SEQ ID NO: 24 - DNA, 21 bp, Dianthus caryophyllus
tcagaagcaaggagtaagaaa

SEQ ID NO: 25 - DNA, 22 bp, Dianthus caryophyllus
tcttactccttgcttcttgagt

SEQ ID NO: 26 - DNA, 21 bp, Dianthus caryophyllus
acccagtcttcttgattcagg

SEQ ID NO: 27 - DNA, 22 bp, Dianthus caryophyllus
tgaatcaagaagactgcggtta

SEQ ID NO: 28 - DNA, 21 bp, Dianthus caryophyllus
agagcggtatcatagtgtacg

SEQ ID NO: 29 - DNA, 22 bp, Dianthus caryophyllus
tacactatgataccgcttctcc

SEQ ID NO: 30 - DNA, 21 bp, Dianthus caryophyllus
ttgcgagattgagagcaaatt

SEQ ID NO: 31 - DNA, 22 bp, Dianthus caryophyllus
tttgctctcaatctcggcaaac

SEQ ID NO: 32 - DNA, 21 bp, Dianthus caryophyllus
tttaaacctcggacgtcaatg

SEQ ID NO: 33 - DNA, 22 bp, Dianthus caryophyllus
ttgacgtccgaggtttgaaaaa

SEQ ID NO: 34 - DNA, 21 bp, Dianthus caryophyllus
ggaagggttgcgtttggaagt

SEQ ID NO: 35 - DNA, 22 bp, Dianthus caryophyllus
ttccaaacgcaaccctctccac

SEQ ID NO: 36 - DNA, 21 bp, Dianthus caryophyllus
ggctttttcaaacctcggacg

SEQ ID NO: 37 - DNA, 22 bp, Dianthus caryophyllus
tccgaggtttgaaaaacgccaa

SEQ ID NO: 38 - DNA, 21 bp, Dianthus caryophyllus
cccctgcttctgcctccaagt

SEQ ID NO: 39 - DNA, 22 bp, Dianthus caryophyllus
ttggaggcagaagcagggggac

SEQ ID NO: 40 - DNA, 21 bp, Rosa hybrida var. osiana
cttacgtggtttgctcacatt

SEQ ID NO: 41 - DNA, 22 bp, Rosa hybrida var. osiana
tgtgagcaaaccacgtgaagtg

SEQ ID NO: 42 - DNA, 21 bp, Rosa hybrida var. osiana
gaaagtgattgggtggataat

SEQ ID NO: 43 - DNA, 22 bp, Rosa hybrida var. osiana
tatccacccaatcactattccc

SEQ ID NO: 44 - DNA, 21 bp, Rosa hybrida var. osiana
gagctgatcaggagcttcaat

SEQ ID NO: 45 - DNA, 22 bp, Rosa hybrida var. osiana
tgaagctcctgatcaggctcca

SEQ ID NO: 46 - DNA, 5229 bp, Rosa_hybrida_osiana
ctctctctctctctctctctatccagtacaagatataactatgtgcataaaatatataagctttgtggctttcattggaggtcctctgagatctactgcttgtgtaagacttggattagatagagattggcagaggaactgaacacaaacaatataacagaaaaggaaagctcagagccttagaggaaggattggatatctgaggcagggtatcaagtgactggactagttggagtttgttgagtgtctgttagttgtgctttgtaattgccagtagcttctcaaggattggattgaccatctaactatagtcaggagataaaaagagaatttgatggggtaaacattctaccctgcagtggcatccaattgggaatgattgaaagcatcagattagtgtctgatgatcacagttagctcttaatgattgaacatgtagtagtcatgaaaaagttgtttgatctgcgcaagtgtattggaatagagtgcgcagccaccttatcaggaccttcattggatgtactttctttaggcatttatgttatccagtgacagtaacctagttcatgtattgtggtgtaagtagctgttactagtgagcagtgtcttgtcacatgggttggaacactgagaatccaacagtcgtgcagtttatctagggttgtactcaattggcgatcctgtggcttgaaagtgtttatcactagttctcctgtattagttctttatgccctcatcattcctcttcgttagtgtccccaaattctaggccctcttttccctgttgtgctcttgtcactcttcatctatctcatcattaaagttcttccatctttccttttcttcacaaggagcggctagctcagttggcaattctagttggttcagtaattcaggaagtccctccgcaagctgaacagatccatcttattcccacaaaagaaaatcattgcaccgccaatttcgcatgtccaagagtctgatagctcatatggaatctgctagctctaacgctaacaatatgccaggcgttgtccatcagttgcttcctgttgttggacccatgcttctgattgcagttggatatcttgaccctggaaagtgggcggcaactgttgaagcaggttcccgttatggaactgatctggctgcagtaatgcttatattcaatttggctgctattttatgtcactatctgtcagcacggattgctgtagtcactggaagagatcttgctcaggtttgcagtgaggaatatgacaaggctacatgcatattcttaggagtacaaacagagatgtcggtgattgtgttggaccttaccatgatcctcggtatcgcacatgcacttaatcttctgtttgggtgggacttgttcacatgtgtgtttttgactgctgctaatgctgttttataccctcttttttccaccctcctggagacttgcaaggcgaaattcctttgcgtatgcatatacattgcaggatttctactgctttcctttgttcttggagtatttatcagtcaaccacaagtgccactttccatgactgggatgttaacaaaactgagtggggaaagtgctttttcactgatgagtcttcttggagcaagtataatgccccacagtttttatcttcattcttctattgtgcagcatcagcaacaaccaactgtttctaaggatgccttgtgtcagaaccattttgttgccatcttctgcacctttagtggtatttatctggtgaattatgccctcatgaccttagcagcaaatgtattctacacttcacgtggtttgctcacatttcaggatgcaatgtccctaatggaacaggtcttttggggtccaatagtacctgttgccttcttgctagttctctttttatcaaatcaaatcacaacattaagctggagtctggggggacaagtagtcttgaatgattttttaaaactagaccttcctggttggcttcactgtgctacaatcagaatcattgccattgttcctgctctgtattttgtttggagctcaggagctgaggggatgtaccaactgcttatatctacacaggtattggcagccttgctactgccgtcttctgtgatccctctttttcgtgttgctgcttcaagacaaataatgggagcccataagatctctcagttcgttgaattcttggccctcattacacttattgggatgcttggattaaaacttgtctttgtagtggaaatgatttttgggaatagtgattgggtggataatttgagatgggatgctgggagtagtatgtctgcacttctcatcactgcttctgcatcattttgtttgatgatttggctggcagctacaccgttaaaatctgcaagtgctcgattagagaaccaggtgtggaactgggatatgcctaagggtgtatctgagccatttagaaatagggaggagattgatatagctgaacctaattatcatagagatgcaagtgttcagaagcatgaaccatcaccatcttctgggaaggctttggatagagactcggatacagcggttgcaaattttgattttgttctgcccgaaactctcttggagcctgatcaggagcttcaatcaactacctcagaggaaaatagttctcttaatacctttcctcgctctgccaaatgcaataaggaggaaaccacatctgtggtggagtcaattcgcctcccaactgtggctagtgaggtttctgatgttacatcactgggcaccagtactgtgaaagttggatcaacagagccagttgagaagattcttggagttgaaggagacttaccaactgaaaaagatgatgatgagggagatacctgggaacctgaagattccttgaaagaggtttctgggggcaccacttcattgacatctgaaggtccaggatcattcaggagtctcagtggaaaaggtgatgaaggggggagtggtgctggaagcctttcaagattagcagggttggggcgtgctgctagacgtcaactggctgcagtactcgatgagttttggggacaactgtatgatttccatggtaatgtaattcaagaagcaaaggctaggaaactggatctgttgttggggtcagattcaaaggcttcttcctccgcttcctccgtgctgaaagatgataccactgcaaaggaagtttctggatactttccatcagtaggaggcagaggatctgatcctttaatcaactcaagtttatatgactccataaatcagccaaggctgcaaaacagtttagagtcatcatatggtgctcaaaggggatcttcctcattatggtctggccacatacagttgctggatgcgtatggacaaaattcaacctgcagtattattgactcgggtgagaggcgctattcaagtgtccgcagcataccatctgctgagagctgggattaccagccagccacagtacacggttatcagatttcatcgtatcttaatagtaatgacagaagttctggtaacttgaatggtcaaatggaatcaccagccctaaattctgcttcttcattgggtgctggaaactacagagagtcacttgcatttaccatggggcagaagttgcaaaatgggttaggctctggacaggcctccagttttcagaaccttacggtatctcgacatagtccgttgcaatctgaaagaccatattatgatgtacactcttctggaatttctgagaatgtggtaaattcagccaatgcaaagaaataccacagtttacctgacattcaccgcgatctctacatgtctaataaaagtgctcaacgggatgctccccctgggtttgggaaaactaattatgaatcatccttgtatccaaaatctgttgcacggacaggaggtcctttggcatttgatgaactctctccatcaaaagtctatagagatgttctatcatcccaacagaattctaattttggcactggatctctctggtctagacagccttttgagcaatttggtgtagctgataataatcgttctattgggactgcagttggtagtagagcaggttctgcaggtcaggaagctatgtcagttgcagattcagaggcgaagcttcttcagtcttttagacactgcattgtgaagcttttgaagttggaagggtctgactggttgtttagacagaatgatggagtggatgaggatctaatagatcgtgtggctgccagggagaaaattatttatgaagctgaaactagagagattaatcgaacagttcacatgggtgaatctcagtacacttctgacaggaagtcaacttctgcaataaaaatgagtgatgcaaatctcacccatctcatggtttcctcagttcctaattgtggggaaggttgtatctggagatctgatctaataataagctttggggtctggtgtatccatcggattcttgatttgtcccttatggaaagccggccagagctctgggggaaatatacttacgttctcaatcgacttcaggggattattgattctgcgttttcgaagcctcgttctccaatgtctccatgcttctgccttcaaattgcagcagcacagcagcagaagtccagtccaacattttcgaatggaatgttaccccctgctgcgaaaccagccaggggaaaatgcactacagctgcaacactcgcggacataatcaaggatgtggagactgcaatctcttgtcgaaagggccgaacggggacggcagccggtgatgtggctttcccgaagggaaaagaaaatctggcatctgtcctcaaacgctacaagcggcgattatccaacaaacctgtttgcactcaggaggggccttcccgttcacggaagggtgctccaacatctgctccttatgggtcataactttcacatacacatcacagttgatctcatctgggttattgagctgtttttgattttgggacaacggctcatcttttggatcaaactgctcacgcgaagcagagaagggctgctctttcggatcaaagggcttagtcaattcaaattctctcattgtatcaaagccattgctgcaaaattgtatttccttatatgttaacatatacatagggtaagaacagctgaatctgtaattgcagcctcatcaaagaaaatgggtagaaggtgaaaaatttggttattgcaatttttgtgttgcttgcttctgttctaataaaatgggaagtttg

SEQ ID NO: 47 - DNA, 2364 bp, Rosa_hybrida_osiana
ttcttcttcttcttcttctgcttgttgtctcagacaattcagtgatagagaatcagaaaagcccaaatcttttttagggtttggtatccctgcatgcggattaggatcaaatgggtgaccttggggaggttggacctgaaattagttcggatatagaagaagacttgaggtgtgataatttagtggagaaagatgtcagtgatgaggagattgatccggaagagctggagagacggatgtggaaggatcgaatcaaactcaaaaggatcaaagaaaaagaaaaacagaaaattgaggctcaacaagctgcggaaaggctgaagcccaaacagaccactgatcaggctcgaaggaagaaaatgtcaagagcacaagatggaattctaaagtatatgttgaagctgatggaagtgtgtcaagctcgtggatttgtgtatggtatcattcctgagaagggcaagccagtaagcggtgcgtctgataacatcagagcatggtggaaagaaaaagtgaagtttgataagaatggccctgcagccatagacaagtatgaagcagagattcttgccatgactgatgcggacaataaccgaaatggtaattcccagaccatcctccaagatctacaagatgcaactcttggttctctactatcttcattgatgcaacattgcgacccccctcaaaggaagtatccattagaaaaggcagttccgcctccttggtggccgacaggaaatgaggattggtggatgaaatcagggttaccctgtggtcagagtcctccttataagaagccacatgacttaaagaagatgtggaaagttggggtgttaacagctgtgataaagcacatgtcccctgatattgcaaagataaggcggcatgtccgtcagtcaaaatgcttacaggataagatgacagcaaaggagagtgcaatttggttgggggttctaagtcgagaagaagccctcattcgacaacccagtagcgataatgggacatctggcataactgagatgccacgtcgtggccgcggtgaaaagcatgctactgctagtagtaacagtgattatgatgttgatggtactgataaggagctaatcgatgcaggatctgtttcatccaaagatgacaggaggactgagctgatggatgtagagccatctagcaatctacgcagtggtactcctactaatcatgtccaagataaagagagaggtgaaaagcgaagaaagagaaaaagggctcctgtaagaccaagctctgatgataaacttcctgcaccaagtcacaatgagcctttgcatgttgaaccattaaatgctttgcctgatataaaccacactgatgcacagatgattggatttccaattcatgaaaatcaacaggaaaatgtttcagtcacaactttacggccaccagagcaagatcttgatgtccaagcattaccggcgtccgagtttaactactatgctgatgtacctcctgatggtgtaactgcgacgacacagggcatgcatgtgggtggaacaccgctgctttatcatgggatgcaaagtgctgagatgcatcatggaaatatgtataacctttataatccatcagcagaacatgtgcccggtcatgataggcagctgtctcagattggcatgaatgaactgcaaattggaccagcagatgttgtccctttacaaactataagaaatggaaatgagattactggaggagatatgccattctatgcaaaagacccatttcagagtgcgcaagacagaactgtagatgctaactttggctctccaattgacagcctgtcattagattatgggggtctatttaacagcccattccgacttgaccttggaattgagggcactggttcattagatgacctgaatgtggatgaaatgatggcatactttgcagcataagttgtaagatttgaagccacatagcttagatagtaagacatatttctctcttatatgctaccttatttataaattgtgtcagaatttgatcaagattcttttttcttttctttttttccttgagaacttcaaaatgttgcattaatggccccctgggatagagtctgttgccggaagttatgcttttagtattttgtagaactcatcattttgctaactaagtgaagggaatgatgggatttcgtgttacagtttcctttcacttatcttcaataagaatgaaaaccctggggttcatgtaaatagtcaattttttaggctggataaatgtcaaatgtatgattatttgctcatttctttggtcaagatgtctaatagatatcctcagtgttgtttctgttatttctctcttgcattaacagtgcatact

SEQ ID NO: 48 - DNA, 2694 bp, Rosa_hybrida_osiana
aagagataagcaccattaaaaaccagtcaacaaatcggaagaatccactacattacatgatcatggttgggaaccccattacattaaagacaattcaatcaatctgaagaacccattacatgacagccttacacgaggacaaggacaaggacaacaaactcagatttctaaccatcaccatggtcgatctggattgaaagaaaattgaagacagaaacaagttaaacaaaccaacctacttattttatatcacagttaatatttatatttataattaacaaaaggaagggtagctcatacaacccttcccccttcctccatttattggcctgctcattccttctatataaggcttttaaccttgattcattctactagagtacatgtgaaatcatgatgaataagtagcaacactctaactcaagtaaggttttagcacatcaagtatattacccacgaatctcgtgtgcagatgtgggctagataccctgcgtaattctccgtttcactggaaccagattgaagtctccgactgcttctccattggcaatgccagcccttgtagatcttctttgtaatcaaaagaggccaagtcaaatggggacccaaacatcataggaaagttactgttatggttggtctcaaatggagaactcacgaccttaaactgctcgaattgaccttcctctcgaggaaacaagtgatgattactggagatgttggattcttcaaagaaattcccatccaacctaactccttgaccacggaagtattcatcctgttgatgtgcaactttaggctgagaaaagttctgaccattggtcgctgaactgttcggatttgcattcttgtcaccttgaacattactatcatagaaagacataagctcgctgatcattttctgcccatcctctggaactcctcctgatagatcaaaggatggtggcaccggcttggcggaaggagctgctgccttggtttggacaaaggattgagggaagatgactggtttaatctcattaacaaggaaattagatgccccgaactcagaagacctgttactgaatgggcaactcaattgatgattgtccctggaagttctgtcatgaaaaccatgacggaattcgctgtaaggacactgaacgaactcacaggtgtagatcttctgatccaccaccatgtttagatcgcttgaaggcttccttttcctcatgaaatccaagtttgtgatgacttctcctttaactggaaaagaagtgggttgatggacttgaagcccttccctcattatttccatcccaaaattcgatgaatgaagattatcaggcttacactcctgaacatcaaagtttggctcatcttcagccccttcaacatcatactcactgcagtcattaatcaccaaagatccactggctccagctgtagacaaaggggggcatgaatttggataaagctctcgagccagggactcttcttggttgatgatggccagccaggtagaactctccttagctgtcatcttgtcttgcaagcatttagattgcctcacaagcttacgaatcttggcaatatcaggggacatatgcttaataactgcagtgagaacacccaccttccatgcctttttcaagtcatgtggcttcttgtatggtggaggaccctgatccttgggcaaacccagttcaggccaccattcctcattcgcagtgggccaccagggtggtggaacacctttctctaaaggaaaccgcctctgtggaggatcacaatgctgcataagtgctgacaagagagaacccagagtggtgtcttgaagctcttgcaaggtgtgaggggttggaccaattgggttgcacccatcgttcctgccgggaactgcattatcagcttgatacttggatatagctgctggtccattacgatcaaacctgaccttgtccttccaccattcacgaagattgtctgatgccccagtaactggttttcccttctctggaataataccatagacaaagccttgggctttgcaaacttccatcatcttcaacatgtacttcaaaatcccatcttgggccctcgacatcttcttcctccttgcctgctcttgagactgcctctgctttgcagtgacaatcacttccttaccacccttagtcgtctgttctctgagtctcttgagacgcattttatctctccacatccttctctcaagctcatctacatctatctcatcatcagtataatcatcctccacggtggcctcggtttcagcctgtgggatggccacttctcccccaggagttgtagatataaaatctagatcaccacaaaatcccatttcgtcgaacatcatgatcatttcccaaatgaaaccaccaaaaagctcacaagaatttcaagtcggtattctggagtctagcttcaatctcaaaaagattcttttgacctgtaaaatctaaaggaaggaaggaggggatgaactcaggagaaaggtctagttcttctcttccgggtcaatattgagggatcaagttttcagaaaagagaggaagagagataagcattatctcttcgtgtattggtattgagtatagtctataagagtattgacccaggtagtacatattttaatgtgggtattatttcagtatacgaataaacaaacaatagaagaagaagatgatgatgatg

SEQ ID NO: 49 - DNA, 1565 bp, Rosa_hybrida_osiana
catttgtggctcttcagcctgctgctgcttcctcttcttaccaaaatccgagttggtctcaataagctctcccttaatctgaggtctttgtccaatacttcccatattgaagtgattcagaagaggtttgcaatcctcaacttcaacattctgttcatcatcaaccccttcaacatcatagtcactggtaccactgattacgaaagatccactcccaccagcagataaaggtggacacttatcagggaacatcttcctggccataacttcctcctggtgtataatggccagccatgtggcactttccttagcagtcattttgtcctgcaaacatttcgactgacgaacaagcttgcggatcttgttaatatcaggtgacatgtgctttatcacagctgtgagaacactgaccttccatgccttcttcagatcatgaggcttcttgtatgggggaggtccctgattggccaagttcaattgaggccaccattcctcattaccagttggccaccatggtggggaaacacccttctccaatgggaaacgcctttggggtgggtcacagtgctgcatcagggctgacaaaagtgaaccaagagtggtgtcctggagctcctgcagggtgtgtggagtggatgccactgaaatgcagtcttccatcaaaccagggattgaattatctgcctgatatttggaaatggcagctgggccattacgatcaaatcttactttgtccttccaccattctcgaagattgtcagaggcaccactcactggttttcctttttcaggtataatcccataaacaaaaccctgagctttacaaacttccatcattttcagcatatatttcaggattccatcttgtgcccgtgacatcttcttcctccgagcttgttcctgtgattggcgctgccttgcattgtcagctccttcctttcccttattttgttctctaagccgcctcaatagaatccgatctctccacatcctcctttcgagctcatcaacatccatttcttcatcactataatcctcctctacagttgcctctgcttcttgctctagaaccacctcaccttcgccagcagcagctgaaaggaaatcaagatttccacagaaacccatttcctcaaacatccccatttcgcgacgaccgaaagaacaaccctgattatataattttatagtaatctgttacttagatgccgtccctgtcaacctcaccaacctcttacaatcaatttcaatcttgcacacagttaccccaagaagtaaacgaattgtatttgttcagattgataatgaagccactgaagctcctgacagcagtctctgattgctcctctcttctttgctcacagagagatacagagagagagagagatagagatgaaaggacagttctttttagtgggttttctttttcttttcttaatggaaggacgaagaacaagaaaaataagagagagagacgggggtgactgcagggacaaagctaggtggaacaaagctactacagtatccaaatttcttctctttagagagagagagagagatgcaga

SEQ ID NO: 50 - DNA, 1077 bp, Rosa_hybrida_osiana
gcagcaggctgaagagccacaaatgatgcttaatcagaaggtttacacctgtgaattcctgcaatgcccatatagtgattatcgcattgggggcttttgtgacattactgctagaaacaatcatcagatgagttgcccatacaggaataatttttcaccagtgtttgcaatgccaaacttgcttgacaatgacaaacctgcaggcttccctctaccagttgctcaagctaagccagcagttatacaacaggtgaaccagacaagcccttacaatgtttcaggactcggacttgcagatgatgggcagaaaatgatatccgagcttatgtcatcctatgatagcaatactcagcacaacccgaatcctggcaacctcaatgctgtacaagaccacaaccaccagcaggcaaaatttcagtatccagtgaatgataacttctatggtcaagggatggttatggatcgtaacaatgagcctgaaccaacaccgatacccatgcttcaacaagttttcccatcgcctgaagttcagtttgatcagtgcaaggtatttgattctgcctttggtaacaatccgaatgatgttattcctgacttgagatttggatctggatctcagtttagcttggcatcgccggactataatgttggtaactcactgctgaagcaagatgctgcatcatcgttttggtacatctgaacaagtttatgtcggcttctccaatatatggatgagtctgtatattatggggggaaacggttcatcttctatacacagtatttctttatctttctgttaaggttttagttacttagttgttatatagttatatatgtccccggacttgttagatcctaagaaattggggggtgtgtcacagcaagtttattgttcaagtatagttggttaagtaggccaataaaaaggaccggaaaatcccaacctagctgattggcaatcagtgcttttgttagaggcattttccggttcccaaggtcaatattggcaaccaagtattattgttgtgttatttatatacacagacatttatttatatgatgcttttcttttatcac

SEQ ID NO: 51 - DNA, 1926 bp, Rosa_hybrida_osiana
tttattcaaatcactaaataaccacacttgtcattaactagtaaattaagaacatacaacaaaccgattatcatggcatagcatgaattacacaagtcaagattgcactgatcaacgatattcatatcattctggctagggttagttgcccaatctatacatggacaacctacatatatgaaacacatctgatatcccagatgaaaaaacctttgagcaagcagcatcagtacccaaagcaattaacgggaaaaaaatttatcatggcctagcacgcataaagtttaaatcaagcattactaatcaacctcgaattatttactagaatagatagctagctaggttgagtggatgagttatacttttttaacttgctcgaagcagaggcgagcgaggcattggtgaatgaggagatatcatacgaggggacatgacctcttcatatcttcttgttgctggaaagctaagcctcagtccttcttgccgacggttcttcctaggactgttcacaaacagctggtgatctgcattattactcttcttgtctccttctaatcccacgaatgctctaatcctcttcaatgcaacttcaactgtatgctcatccatgttcgcgaagcaaaccctaaaccaaccgggctccacgcaacggaaggaagaccccggcgaaacgttgagcttcactttgttgataatcatacgccacagcaccatttccgcctcgaaagtttggtctttgagcagcttcctcaagtccatccagcaaaagagcccggcattgcctttcaagcagttgatgcctacctcctcgagccccgtggtgaaaaaccggtgcctcctcgccagcctcttagagctggtctcaaggaagttggagacaaattcatcgtccagaagcatggcggagagcatgtgttgagtttgggaggagaccaagccgaaacttgacatcttgcgagcgcagttcacgacggcgtcgttgtaggagtaaacgatcccgatccttaaaccggggacacccatgtccttggacaagctatagacaatgtgaatcaaatcgcggttgcaattcttcacgtcttgtaggacctctgtaacgcaagtgaattttgggcagctgaacactgtgcctgcgtatatttcgtcgcagaccaagtggatgttcttgtcattgatgaatttgaccaagctagtgagtgagtctctgtctatggttgtccccaatgggtttgaagggtttgttatgatcaagccctttatgttgatgttgttgtttcgggccttttcataggctgcttcaagtgctgctctggtcacttggaaattgtttgagctatcacaatcgactggaacaattttcactcccgttcgccaccccaagtctcggtagaacgctggatagtaaggagagggaactaggaaagcatctccggggtcagccaagcaaaacatgaccgtctcgttagctccagtggctccgccggccatgactacgcgatcaggatcaaatttgactctgcctcctctcgcctttgacatgaagttagcaatagcctttctgaactctggaaagccatgatagtcttgaaagttggccacatttttgaactccgcaactccttcaggagtgcaaaaggaggccttgggatttttcttaatccactcttcaatcacatcgaaagaaagctgattttctgcaagacccatctgaataacaccctcggggttctgagtcgggtgaaatgggtttccgtcatacgccttccatccatcgaagtaagccaagttctcgccatgtccatcattggttgcaatctttgacaagagtgagttcgaggccattttctttgttgctctaaacgtcgtggtaagtgtataatcaagatctaaattgggttgtgtcccttgtactgttgtttggatttg

SEQ ID NO: 52 - DNA, 1655 bp, Rosa_hybrida_osiana
ccagaagtctctagtatattcacattttggtgtaaaacaatgtacaatgaagtgaaagcactcaaagctctttgatggaaaattattacaaaccaagacaaacttttttcactgaaagtgaaaaaagaaaaacctgcaatctgcaggtgagaaaacaattgcatgtattgactagcttctaggtggaatacctattcttggttcttttcttgtgttaatttacgagtatatccatccacctagctaacaattaacctataaagaagtacagtaactaagagaaaacatgcatcaatgttgctagctgcatatctaaatggagatgatcatcacttttttaagtggctcgaacaagaggtgactgtggaataggggaatgcggtgacatgctgacctcatccaatattcgaaaattgaaggatttgaagctgagactcagctttttattactttgccagcagctcttcttcttaattgctaccacagcatccttgtcttgaagcacaaagtttctgattcgttccaaagcaacttccatagtctggtcgtccatgttggcaaaacaaaccctgaaccaacccggttcggggcaatgaaaagaaacaccgggtgacacattgagcttaacttgatggattatggttctccacaatgccatctctgcttcaaaagtttgctcctttaaatgcttgtgcaagtccatccacacaaacaagccaccattgctcttcaaacaagtggtaccttgttcctcaagtcccacagtgaatctcttgtgccttgctttcaacctcttggcactttctacaatgaatctgtccacaaactcattgtctgatagcatcgatgcgattagatgctgagtttgtgtagaaaccaacccgaaactcgacatctttctcgcgcaattcactactgcatcattgtatgagtacactatcccaaccctaaagccagggaaccccatgtccttggaaagactgtagacaatgtgaaccaggtcccggttgcattctacttcttcctcgagaatttcagcaatgctgatgaaccttggctgagtgaacacagtggcagcatagatctcatcacagactaggtggatattcttctcattgatgaaagttactaggcttttaagggtctctctgtctaggactgtgcctagtgggtttgaggggttggtaatgagtaagcctttaactctgatgttggccttttgggctttctcataggcatcttccaaggctgctctggtgatcttgaaattgttggagctttcacaagcaactggaagaagttgtacccctgttcgccatctcaaatctctatcgaatccaggataataaggaacgggaaccagaaatgcctctccgggatcagccaagcaaaaggcaatcatctcgtgagctccggttgctccgccgctcataacaatgcggtcagggtcgaatgtgactcgatttcctctcacttttcccatgaaatttgcaacagcatttctgaactctggcaggccatgatagtcttgaaagatggctatgtccttgaatgcttttgctccttgtgcagtgcaaatggaggcttctgggtttttcagaacccattcttgaatcaaatcaaaagaaagttgattttctgcaagacccatctga

SEQ ID NO: 53 - DNA, 1790 bp, Rosa_hybrida_osiana
attgcctccgcaacaacaaagcagacacttccctcccactcaagccactctcactctctccaaccaactcctccggcgccaccacacactccccaaacccatgacccgaacccgcaaacccgaaacctcctcctccgacgaagacaacaacgacgaaaagcccgccgcatccaccaccgccatgagactcatcgtccctctccaaggcgtcgtccaaggccgcggaggcctcatcctcggctctctcatcccctgcgctctcttctacttcctccagctctacctcaaacgacaccgtccctcccaacccgacccggacccgccttctccctcctcctccaacctccccgagctccaccgatcctcgtcgcgctccagcctgtccggccgcggatccatcggccgggtccgggtctcctcccgggccgacccgattgccaagcccaacgagtcgccgtattatatcgggctggatagggttctggatgacccgtatcatagtgtggataatccgaatggggttattcagcttggattgtctgaaaataggctgtgttttgatttgattgagaaatgggtgtcggagaattggacggaatcgatattgggagccaacggtggtgatttgagcattgccgggattgcggcgtaccagccgtttgatggatttactgagctgaaagtggctatggctaatttcatgtcccaggtgatgcgaagatcagtgccatttgatccgtcgcaactagtgttaacagctggtgcaaccccggcagttgagatattgagtttctgcttagcagaccatggaaatgcatttcttgttcctacgccgtattatccgggttttgacagggatttgagatggcgaacaggagtggagcttattcccgttcactgtcgcagcactgacaacttcactttaaatataaccgttcttgaacaagcatacagtcaggctagaaaacgaggggtgaaagtacagggaattttaatttctaacccttcgaatcctgttggcaatttagtttcaagggaagcactgtgcagtcttctagactttgcccaagagaagaacatccatattatctctgatgagatctttgctgggtctatgtatggaagcgaggagtttgtgagcatggcagaaattgttgagacagaagattttgagaagaacagagttcacataatatatgggttatcaaaggacctctctattccgggctttaggattggggttatatattcatataatgatagtgttctggccgctgcaaaaaggttaacaagattttctcccatttctgctccgacgcagcgccttattatttcaatgctcttggatactggattcattcaggaatacctagacatcaacaaaaggaggatacaacatatgtacgatttatttgtggatggtttgcaacaattaggaatcaagtgtgcaaagagcagtgctggtttatactgttgggccgatatgagtgggttaattccctcctatggtgagaaaggggaactcgaattatgggataacctgctgaacattgccaagatcaacgtgactcctgggtcagcctgccactgcatagaaccaggatggttcaggtgttgtttttccagcttgatgcctgaggatgttcctgtagttatagatcgaatccgaaaagttactgaaacaattaaatcttccagttgaaattttttgtttgtgtagcgacgctgtttgaatgtttatggtattgcttatagaagtagctgatatagcctgaagaggtagatgctgt

SEQ ID NO: 54 - DNA, 2013 bp, Rosa_hybrida_osiana
caaatgaaagccaaacccaaaaaggctatatcagtataaactctccccatcaatcccaccaaaaaatcccaacgactggttccaaatcctcgcctcaaaccaaggaagaaacggcgcccatatcaaattaaatctcaaaacaaaacaaaaaaaacaaaaaaaatctcaatataccctccaaacatttcgctgctctctcactcactcactcgccccaaagccttggcctttcctcccttcgctttcttcttcctcttcttcatcatcgtactctccgacgacccgaaaccccaccgcgacccggcccggatgtctccaatatgacccggacccgagacgaagaccggcgacccaccagcagcagcagcggcggaggcgccgccatgagagttatagtccctctacaaggcgtggttcaaggcagaggaggactcgttctcggctccgtcataccatgcgcgctcttctatttcctccagctttatctgaaacgtcaccgttccaactccaacccgccgactccgccgccttctccggactcggactcggaccaccacccggccgggcagttggtggaagttccggttctgccccggtcgctgtcgaggtcccatctctcgccgaggaacccgggtccggtacatgtctcgggtcgggccaattcggttttgaaaggcggtgagccgccgtattatgtcgggttgaggaaggtggcggaggatccgtacgacgagttgggtaacccggatggggttattcagctgggtttggatgaaaacaagttagctttggacttggttcgagattggctactggagaatgcaaaggatgcaatactgggtggtgaggagcttgggattagtgggattgcttgttaccagccttctgatggtttaatggagctgaaactggctgtggcaggattcatgtctaaggccattggaaattcagttacgtacaacccctcacaaattgtattgacagctggtgcaacccctgcaattgagattctaagcttctgcctagcagacagtggaaacgcatttctcgttccggcaccatattaccctggtttggacagagatgtgaagtggcgaactggagtggagataatacctgttccatgccgcagtgctgacaaattcaatttaagtataactgcacttgatcgagcattcaaccaggcaaagaaacgtggtgtaaaagttcgtgggattataatttcaaatccttcaaatcctggtggcagtttacttactcgtgaatcactttacaaccttctggactttgcccgagagaagaacattcatataatctcaaatgaattgtttgctggatccacgtatggaagtgaagagtttgttagcatggcagaaatcgttgatttggaagatctcgaccagaacagagtgcatatagtatatggcatatcgaaagatctctcacttccaggtttcagggtgggtgccatctactcctttaacaagaatgtcttgactgctgctaaaaagttgacaaggttctcttctatctccgccccatcccaacggttgcttatctctatgctttcagacaccaaatttatgcataagttcatcgagattaacagagaaaggctccgtggaatgtatcttagatttgtgacaggattgaagcaattgggcattgagtgcacaaagagcaatgggggtttctactgttgggcagacttgagtgggttaattcgctcttacagtgagaaaggggagcttgagctctgggataggttgttgaatgtaggtaagctcaatgttactcctggatcttcttgtcattgtattgaaccgggatggttccggttttgttttacgacgttgactgaaaaagatatccctgttgttatggaacgaattcggaatattgccgaaacatgtaaatcacacagttgaaatgttcgttcattctacacaagtacacaggttcaggttgcatacaaatttttaaaggaaatagcttttactatatctttagaat

SEQ ID NO: 55 - DNA, 1381 bp, Rosa_hybrida_osiana
ttaattacaaaacctctctacagtccaatacagtgagaactttcaagtttcaagaacatgggtccctgaaatttttggcattttgataattatttgtatttattccatattgtttctatgacagtacatcaaaaacactgatattgatgttattcaccagaaagtcccaaactagcttatagctctattaatttatactctaaattcccaccagacaaggaggaaattcaggtagataaaaacgaaattaactgaacttaacaaccttattacaaaagtaactacacacagaccaacatacaaagtcaatcgactgatttcagggctcaaacagttgcaattgattccatagccttcatagcttcaaacctcggctccttggcttggaatttgaggcctgaatagagcttcatgtagtcatcaaacacaaattttggataagttgggcattcctcagtttgtttttcaagcattgctggtgctggagagataaaagcatcattgcctgggttgtagaacgaagctatcgacattctgtttccatcaggttgcgctatcacacggtgcatcacactcttgtacttgccattagtgatcacctcaagttggtcacctaagttgatgacaatggagtggtgcattgggggcacatcaatccattggtcatccttgaggagctggaggccgctgaccttgtcatcttggaatagtaggatgatgccaccggcgtcggtgtgggcccggagtcccttgatcaggtccggtttggggcatggagggtagttgctcaccttggtcccaaaatttggtccctcggatccatagaaagctttcttcaggtaacccttatccagccccagattctcacacagcaagtccaacagttgctcagccagtttctctagttccactgcaaattccttcatggcctccctgtaatcttggtcgagatcggggatttgggaaatgttagagaaggggaggtggcgcaagaagaaggtgctttcccagtccaaatctttgatttcagagttgacagattcgaggcctttgcttgccaccatttccttgaacctttgctccatgcactttctatagtgctcctttgtgagcttctctaccttgtccatcagctcatgggatatcccatggttcaccaactcaaagaaaccccaattctcacaagcatcgtttatcttctccatggttgcttgtctctcttcaccgttgagttgctccatgttaacaaccgggaaattctccatttcgatctgccttgctttctatctttgatgtcttgatgtctctctctctggacctttctagctttcagatgaatgttgagtggagagttctagcaatgctccaatttataggcgtt

SEQ ID NO: 56 - DNA, 1543 bp, Rosa_hybrida_osiana
acccattgagcttctggacacaggattaaagaattgagcaacaaacactagttgagagagagagagaggttgagagagagagagagaagtcgagaaacatggagaacttcccagtcatcaacttggagaaactcaacggtgaggagagaaaagctacaatggaaaccatcaaagatgcctgtgagaactggggtttctttgagctggtgaatcatgggctacccattgagcttctggacacagtggagaagatgacgaaaggccactacaagaattgtttggagcaaagatttaaggacctggtggccagcaaaggccttgaggcagttaactctgaggtcaaagacatggactgggagagcaccttctacctgaagcaccttccccactcaaccatctcagaagtcccagatctcgaggacgagtacaggaaggtcatgaaggagtttgctctgaaactggagaagctagcggaggagctcttggacttgttctgtgagaatctgggactggaaaaaggttacctcaagaaggtcttctatggttcacagggtagtcctacctttggcaccaaggtcagcaactaccctccgtgccccaccccggacctcatcaagggtctccggtcccacaccgacgccggcggcatcatccttctcttccaggatgacaaggtcagtggtcttcagctcctcaaggacggtaaatgggttgatgtgcccccaatgcgccactccattgttatcaaccttggtgaccaacttgaggtgattactaatgggaagtacaagagtgtggagcacagagtgattgcccagacagatggcaccagaatgtcaatagcttcattctacaaccctggaagtgatgcagttatctacccagcaccatctctggtggagaaagaagcagaggagaagaatcaagtgtacccgaaattcgttttcgatgactacatgaagctctatgcaggcctcaagttcgaggccaaggaacccagatttgaagccatgaaaacagttgaagccaatcccagtttggctgcaattgccacagcttaagggcaaatttaatcgatctactcaagttaatgggtgtgaaagtatcgagcaaagcttttacttgaactagattaatttcaaggtttactattactgtggttatcgtcggtgtgtggtttgtagctagagattgattgatttaccaaggttactattctatagtaatatatattttgacatgggaatttgctatataaataatctgttgcaaatccctaagcaataattaaaacccgattgtgcttaactagattctgtcagttatataatctctcaaacttgtcaacttataaaaacaaggcctccaaaatagcaaaatcacagtgataagctaaagatgctaaagccactcggcagcagtgacatgccgaatggcttcagccacatgccgacatgatgaagctaaagtatttttaaaagtatgagttcaattccacatcaatcaaaggcacaagaagaggactagaggagtataaaaataa

SEQ ID NO: 57 - DNA, 553 bp, Rosa_hybrida_osiana
atgctgaagatgatggaggtgtgtcaagctcgtggatttgtgtatggtatcattcctgagaagggcaagccagtaagcggtgcttctgataacatcagagcatggtggaaagaaaaagtgaagtttgataagaatggccctgcagccatagacaagtatgaagcagagattcttgccatgactgatgcagacaataaccgaaatggtaattctcataccatcctccaagatctacaagatgcaactcttggttctctactatctgcattgatgcaacattgcgacccccctcaaaggaagtatccattagaaaaggcagttccgcctccttggtggccgacaggaaatgaagattggtggatgaaatcagggttaccctgtggtcagagtcctccttataagaagccacatgacttaaagaagatgtggaaagttggggtgttaacagctgtgataaagcacatgtcccctgatattgcaaagataaggcggcatgtccgtcagtcaaaatgcttacaggataagatgactgccaaaatcactagtgaattct

SEQ ID NO: 58 - DNA, 539 bp, Rosa_hybrida_osiana
atgctgaagatgatggaggtgtgtaaagctcagggttttgtttatgggattatacctgaaaaaggaaaaccagtgagtggtgcctctgacaatcttcgagaatggtggaaggacaaagtaagatttgatcgtaatggcccagctgccatttccaaatatcaggcagataattcaatccctggtttgatggaagactgcatttcagtggcatccactccacacaccctgcaggagctccaggacaccactcttggttcacttttgtcagccctgatgcagcactgtgacccaccccaaaggcgtttcccattggagaagggtgtttccccaccatggtggccaactggtaatgaggaatggtggcctcaattgaacttggccaatcagggacctcccccatacaagaagcctcatgatctgaagaaggcatggaaggtcagtgttctcacagctgtgataaagcacatgtcacctgatattaacaagatccgcaagcttgttcgtcagtcgaaatgtttgcaggacaagatgactgccaa

SEQ ID NO: 59 - DNA, 407 bp, Rosa_hybrida_osiana
atgatggaagtgtgtcgagctcagggttttgtttatgggattatacctgaaaaaggaaaaccagtgagtggtgcctctgacaatcttcgagaatggtggaaggacaaagtaagatttgatcgtaatggcccagctgccatttccaaatatcaggcagataattcaatccctggtttgatggaagactgcatttcagtggcatccactccacacaccctgcaggagctccaggacaccactcttggttcacttttgtcagccctgatgcagcactgtgacccaccccaaaggcgtttcccattggagaagggtgtttccccaccatggtggccaactggtaatgaggaatggtggcctcaattgaacttggccaatcagggacctcccccctacaaaaagccccacgg

SEQ ID NO: 60 - DNA, 831 bp, Rosa_hybrida_osiana
gcttgtgaaaactggggttttttcgagttggtgaaccatgggatatcccatgagctgatggacaaggtagagaagctcacaaaggagcactacagaaagtgcgtggagcaaaggttcaaggaaatggtggcaagcaaaggcctcgaatctgtcaactctgaaatcgaagatttggactgggaaagcaccttcttcttgcgccacctccccttctccaacatttcccaaatccccgatctcgacgaagattacagggaggccatgaaggaatttgcagtggaactagagaaactggctgagaaactgttggacttgctgtgtgagaatctggggctggataagggttacctgaagaaggctttctatggatccgagggaccaaattttgggaccaaggtgagcaactaccctccatgccccaaaccggacctgatcaagggactccgggcccacaccgacgccggtggcatcatcctactattccaagatgacaaggtcagcggcctccagctcctcaaggatgaccaatggattgatgtgccaccaatgcaccactccattgtcatcaacttaggtgaccaacttgaggtgattactaatggcaagtacaagagtgtgatgcaccgtgtgatagcgcaacctgatggaaacagaatgtcgatagcctcgttctacaacccaggcaatgatgcttttatctctccagcaccagcaatgcttgaaaaacagactgaggaatgcccaacttatccaaaatttgtgtttgatgactacatgaagctctatgcaggcctcaaattccaagccaaggaaccgcggttcgaggcgatg

SEQ ID NO: 61 - DNA, 563 bp, Rosa_hybrida_osiana
cagctctgcttcgatattctcaagtcgtggctggcgaagaatccagacgcagccggattcaagagaaacggagaatccatttttggggagcttgctcttttccaagactaccacggcattcccgaattcaaaaaggcattggcggaatttatgtctgaaatcagaggaaacaaagtgagctttgatcccaaccacttggtcctcgccgccggtgcaacctcagccaatgagactcttatgttttgcctcgccgagcgcggagaagcatttcttcttcctactccatactacccagggtacgtactcgtttttgtgtacaatacgtctaaatatttaagttttatatatactcaacatacagttactgatcttagttactttcatttcttccagatttgacagagacctcaagtggcgtaccggggttgagattgtacccattcactgcactagctctaatggcttccaaattactgaaaccgctctacaagaagcctaccaagaggcccaaaagcgtaatttgcgagtcaagggcgttttggtcaccaatccctccaaccccc

SEQ ID NO: 62 - DNA, 740 bp, Rosa_hybrida_osiana
atggggttggcggagaatcaagtgagtaactgcgagacaaattaatatcaaatatctgtgaatgaaaaattcattttaagaattttcctctgatattaatttgttatttttgaacaggtttcatttgatttggtggaagagtacctggaacaacatccagaagcttccaacttgggatcaaaagggtttagagaaaatgccttgtttcaagactaccatggccttttgtcttttagaaaggcaatggcaagtttcatggagcaaatcagaggaggaagagccaagtttgaccctagtaggatagtcctcactgctggcgcaaccgcagcgaatgagctgttgactttcattcttgctgatcctggtgatgctttgctagttccaaccccatactacccagggtaagtaccaattcttatctatatattacactttacgctctatatatctccaaagggtaatgttctcttgtcagagtattacatattatgttgactgtctagatcgacagtttcgcaaacataacatgtcagacttccgataagagtgcgtactcatgatcatgcacactttgtatcagttccatataaatatgatcctcatatctaacgttaagtcaaatttcattttttccagatttgatagagatttgaggtggaggactggagtgaacattgtaccaatccactgtgacagctccaacggtttccagattactcctcaagctttagaagctgcat

SEQ ID NO: 63 - DNA, 931 bp, Rosa_hybrida_osiana
gggttggcggagaatcaagtaagtgctagctcatgagacgtaaatcgattgtattattattttgttgagtttgcaactaactaattaacattaatttgttgttttcattcttgaacaggtttcttttgatttggtggaagagtacttggaaaagcattcagaagattccaaccggggatcatcaacacgctttagggaaaatgcgttgtttcaagactaccatggccttttgtcctttagaaaggcaatggcaagtttcatggagcaaatcagaggaagaagagccaaatttgaccctcagaggatagtcctaaccgcaggagctactgcagccaatgagcttttaactttcattctagctgatcctggagacgctttgcttgtcccaaccccatattatccggggttagtagtcctcttacatcgctcaaatttcaatatgtatatatgttttaatttagagcagcgttcacttgctggataccgaagatatagcacatatgcatatatgataacgatactgtcaatctggccagctgcatatttagaacaagagtttttgatcgatcaataaacaggattaacagtttcacaaacataatatgaatttgtcgctagatcatctcgttttgttttttttccttcaaattttggaagctagatatagttatatttcttgcgtatatatagttccatatatgtgtatatattcctaatttctcaagcctaatgaacttaaattcctttttccagatctgacagagacttgaggtggaggaccggtgtgaatattgtaccaatccattgcgacagctcaaacaatttccagattactcctcaagctttagaagcagcatatagtgaagcagaagtcaagaacatgaaagtgagaggggttttaattacaaacccctccaaccccctcggcaca

SEQ ID NO: 64 - DNA, 1346 bp, Rosa_hybrida_osiana
atggggctggcggaaaatcaagtaagagattcaaagacagaaaccttgttttacaagcataatttcatctcttggctatttactaattggtttcttctgtatgcagctttcttttggtttgattcaagaatgggttctgaaaaacccagaagcctccatttgcactgcacaaggagcaaaagcattcaaggacatagccatctttcaagactatcatggcctgccagagttcagaaatgtatgtattgatcaacatctacaagattttcttagcatcactaattaactcattacacaatcttaccaagtttgttgttcaaatattaacaaagttaattattgttgtgaaactccacaggctgttgcaaatttcatgggaaaagtgagaggaaatcgagtcacattcgaccctgaccgcattgttatgagcggcggagcaaccggagctcacgagatgattgccttttgcttggctgatcccggagaggcatttctggttcccgttccttattatcctgggtaagcttaattttatatatctttctaagattgactatgatccaactaagacgggaaaaattagctaattaatttaggcgagttgcctaatattttatgagaaattagcaatgaaagagactggtgttgacccttatgtggtcttggaccatcttgaattattgaggggggaatcaaaacgatctttgtacaaattgtcttggctagacaccattcaatctatacctcactttgataaacatgaaagatgccttgggaaaataaggaccatccttccttttaaaaggacaagtaaaactgtacaattggacacttgtaaacttcttatggtagctaggcgcactgatcacgtcattaaaatatattctttatggagatctgacaagtagattatctactttgatacttatttattgttatgcgaaatttagaattctcgacgtagaattccatatttattcataacgtacggttaaaatattctccatgcatgagttgcttataagtagattatctactctgatacttatttattgctatgcgaaatttagaattctcgacgtagaatttcatatttgtttacaacgtacggttaaaatattctccatgcatgagttgcttgcgaaataacttttttacatcattgcagattcgatagagatttgagatggcgaacaggggtacaacttcttccagttgcttgtgaaagctccaacaatctcaagatcaccagagcagccttggaagatgcctatgagaaagcccaaaaggccaacatcagagttaaaggcttactcattaccaacccctccaaccccttcggcaca

SEQ ID NO: 65 - DNA, 579 bp, Rosa_hybrida_osiana
ggatccctttcaggactaccacggcctgccagagttcagaaatgctgctgcaaatttcatgggaaaagtgagaggaaatcgagtcacattcgaccccgaccgcattgttatgagcggcggagcaaccggagctcacgagatgattgccttttgcttggctgatcccggagaggcatttctggttcccgttccttattatcctggattcgatagagatttgagatggcgaacaggggtacaacttcttccagttgcttgtgaaagctccaacaatttcaagatcaccagagcagccttggaagatgcctatgagaaagcccaaaaggccaacatcagagttaaaggcttactcattaccaacccctcaaacccactaggcacagtcctagacagagagacccttaaaagcctagtaactttcatcaatgagaagaatatccacctagtctgtgatgagatctatgctgccactgtgttcactcagccaaggttcatcagcattgctgaaattctcgaggaagaagaagtaggatgcaaccgggacctggttcacgttctctccagtttctcctgcagtat

SEQ ID NO: 66 - DNA, 1089 bp, Rosa_hybrida_osiana
atgggtctggcggaaaatcagctttctttcgacgtgattgaagagtggattaagaaaaatcccaaggcctccatttgcactcctgaaggagttgcggagttcaaaaatgtagccaactttcaagactatcatggctttccagacttcagaaaggctattgctaacttcatgtcaaaggcgagaggaggcagagtcaaatttgatcctgatcgcgtagtcatggccggcggagccactggagctaacgagacggtcatgttttgcttggctgaccccggagatgctttcctagttccctctccttactatccagcgttctaccgagacttggggtggcgaacgggagtgaaaattgttccagtcgattgtgatagctcaaacaatttccaagtgaccagagcagcacttgaagcagcctatgaaaaggcccgaaacaacaacatcaacataaagggcttgatcataacaaacccttcaaacccattggggacaaccgtagacagagacacactcactagcttggtcaaattcatcaatgacaagaacatccacttggtctgcgacgaaatatacgcaggcacagtgttcagctgcccaaaattcacttgcgttacagaggtcctacaagacgtgaagaattgcaaccgcgatttgattcacattgtctatagcttgtccaaggacatgggtgtccccggtttaaggatcgggatcgtttactcctacaacgacgccgtcgtgaactgcgctcgcaagatgtcaagtttcggcttggtctcctcccaaactcaacacatgctctccgccatgcttctggacgacgaatttgtctccaacttccttgagaccagctctaagaggctggcgaggaggcaccggtttttcaccacggggctcgaggaggtaggcatcaactgcttgaaaggcaatgccgggctctattgctggatggacttgaggaagctgctcaaagaccaaactttcgaggcggaaatggtgctgtggcgtatgattatcaacgaagtgaagctcaacgtttcgccggggtcttccttccgttgcgtggagccaggctggttccgggtttgt

SEQ ID NO: 67 - DNA, 875 bp, Rosa_hybrida_osiana
caaacacaatatcattttctggttttgccaacttgcaaatgaagaccatagttagttgccactcgaagtgcatgtaatggaatttatttacaagatttttactagcttagcctagctagttgcagtagcagtagtgaaataataatacaaccggataaagcttcataatataaaatggggcatgcgcacttagttacgtctatcaaatcatcttctcctttcccttctcatctctctcgatcttgcttacgtttcctatcttagtttggcaaacttatttcatcaagatcatccaaagataagaacacctcgtccaaacatgaaagctcagtcccatcatcgtcctcatcttctccgcgctcataaactcgacataacacgtatccgctatattcattaactgctttccggtgccctcttctcttcgatgatctaggggtggaagaagaagcacaagtatccgagactgctgagagacgatactcatgcattgtccagtttgttttgatccctgaaggagcttttcccacgtagaacccgaagtatcttttgattccaattttgttgttagaagtagaagatatgacaggttcctcggtgcctaacagcttccaatatccactactcgtaacccgattttgcgtcctcctgctatagaagtaccactgcttaccctcagatagagccttaccatttagctcccatggatcgtatggatagagatcgagatcaggaatgacatcggggtggaggggtaagagagctgccttgcgttgaaggaaatggactacgagctcttcatctgtcgggaaaaatcgaaaaccaggagggagctgaaaagggttatctgccatcacaagaaaccagaacaagaa

SEQ ID NO: 68 - DNA, 1012 bp, Rosa_hybrida_osiana
acagttgaaggctaagaattcttttacacccctcttttttgtattctttatctctcccaccagatcctctagtgcaacttcctccaccttcaagtttaagccaaaagatcttgcatgattctgaagttgcttctttctctcgtcaaatcttccttgtagaacacaatcactttcttcatcttcccatttaattgctgtcactctcaatgttttgagtctttgtgctaaagcttcaatcatctgtgacaactgcaccccttctcccatatcaaaatctactacgtgaaccatctctgtatcctcgggaatagactctaggattgctgagtttgccgcatagtgagcaaacttcccgtagggaaagttttggtagaacaacttgaatgcaggctcaaaattcttgtaggattgttgtttcagataatcaacatcgccttgttgatcatcaacaacttctcgacacaagtgaaatgcaagacgatccaaactctgcccaattgggctgactttgtccttgatgcatctgaaaattacctcctctagctctttctgtccatttgccatggattctgcataagccttgagaagatgacgaatacttgattcgttatctatttccaccacttctgcaggcaaagttagtgatgtttggattgatgtcgagtccattgatgatacttcagaagtcaccaatgaatttgacacacaatcaccttcaatagaaaattgtgaggagatttcttgtgaaggaagactcccttcactcccttccaagaaactaccatacaaacagtctatcatcaaattagggtcaaattcttccaaattcattgggaatacctccacctgtgatgagacctgaatcagatcattaccagaaaacaaggaggcaaaagggtctgaacaagtgtcggataagtcagaaaattcatctgagttgatgatcaaagaagaaaaatctgagctacccatttgagtatcatccattttcatggaatcagattgatcaaaa

SEQ ID NO: 69 - DNA, 1447 bp, Rosa_hybrida_osiana
agaagctgcttctacttcttcattttcaaagagaataagaatatgaattggcaaggcttgtgtggaagttcataagatgaaagaattccactgcttttctattcatgcaatggttgcaaacttgcaattaatacactctacaaattacatgcttgacgtcatggatcacaatctcacaaatacatttacatgcatacatacactcatttaaggacaaacaagcaattaagcacatccttgctacgatcattcttctgcgttatctggactcgaagtagtctgtctcaaattgttcttccttctttgtggcgaaatcagtagaaactgcatgtcaagtctaccttagattaggatgcttaatagtgaagccatacatgatccatatatacatataacatactcatgcttggtttgaaggactcaacacatgagtaaaaatcttcaaaccaaatatccatatccaagagctccaaccttttagttaatccagaaatttaaccattgtgaagtagcactgctgctacgcggcggatcaaacagaccgcctgaatcaaatctgtgccaatcttcaggtgacactatagctcctacttgacacatgttcataagctgctcatgaccttcattcccaacatcatcacttgcaaaattcttgtgggactgtaagtgacttggcttttcttctgtagtctcacaatctccgtctttgttacttgagccttccaaatctttaccatccatgttctcttcatgaacttttgattttcccatgagatcatttagcttctccaactgtataagcaaggattgcttttcctccttcaaagactcaaacttagatgctaacaagtcatactcatttctgagtgttctaaagtcttgctcgatttgcttcgacttccatctagctcttctgttctgaaaccatatagcaacctggcgcggctgcagccccagctctcttgccacctgcactttcctccttggctcaagcttcgaatctgcttcgaatatcgactccaacaacttgatctgctcatcactaaacctcctactattcaggctcttgttattcttcttccttttcgcagtagaatgggtctctaggctttctacttccctctccattattaaggtgaatgtgctttggaatcttggcaatgatgttagcttgtagaatatatagttaggaattgaaacaaagtaacgcggttgctttcttgtaattggaactattgattgatggagcgtcttaaccaatggtgttagcaaacgtaagccacgtaatgatctctacatttcatgccttgtggacctctctttcttatcttctttctttcctatttcttcactagaattcaacaaagtgttgctttcattgagttcccatctttagaaatactctacaggcaatgacatacatgcaagctcacctttttggtgtttg

SEQ ID NO: 70 - DNA, 575 bp, Rosa_hybrida_osiana
cacacagaaaaagtaccattccttctctcccattttcgctttatatggcaaatcccaaggctcagacttgttcaagtccacatcaccaattgctttgcagccgaagttggaatcgataaccttcttgtgcaggtagtgagaaataagctcttcatcggttggatggaatcgaaaccccggtggcaaatcaatctgatcatcttccctgaaagttccagcttgaacagcaaacttctccatttcttgcatcattgataccttaaaacctcacttctagctttcttgtcgaaaaccgttttttcagaatcagggttttgatcaacacggcatggttttcaaaataaaacctcaatttctacaaatagagtcctctggatttctcaaacttcaatgagttgtgaccaatacagctcagaacatgaaacagaaccgaggaagatcggaagcaaaaagtttgttgtcaaggagcaaaagggtaaagctcgaggaaaagaagagaagccatggaacaagttgtgagagaaggatgagattttaagagaaagaaaagcaatacgagaaacagaagcccctaa

SEQ ID NO: 71 - DNA, 1800 bp, Rosa_hybrida_osiana
tagtttaggttctggtttacaacataagacacaatcaatctgtacagcaattcttgaggtatgtccaacatgttgaatacatgcagttgctgcaataaattggaagaaaaaattatctacttttatctagcttagctttggttttcccaagttgatactctaaccagaggagttcctctccattccaataccatttcattcccattgagtccttcaatcctgattccataggatctttcatctcccacaatttccttggcttccatcaaattttctttgctcaatctccgaccctcaagtccgaaccatggttgaacatggcagtccttcatttctgtccacttttggaaccaagcctgggaagagacaaaaggtgctatgaagatgcattccatcgccattcttgcttctgccagatgacttgggaaattcaactccattgactccaacaaggcttggtaatgcactagattctcattaaagaatgagctgaaactcgagtgatttccaagtctctcacaagcatccccatcaccgaaagttataataccacttttagtagtcttggagttcccagaattggctaactccttagccagtctcagatactccatgacatgccctctgcttcttctcctcctcatgtgtggaagtgaaaccatacagttgaaggctaagaattctttccctccacccctcttctttgccttctttatctcagtcaccaagtcctccattgctacttcttccacctttaagtttaagccaaaggatctagcatggttctggagttgcttctttgtctcctcaaatctcccgtgtggaggagcacaatcactctcttcctcttcccacttgattgctgtcacttttagtgttttgaatctttgtgctaaagcctcaataagttgagacaactggaccccttctctcatgtcgaaatccacaatgtgaattgcctcagcttccgctggtatcgcctcgaggattgctgagtttgctacatagtgagcaaacctagcataggggaagttttggatgaacaccttaaatgcagcctcaaaattcttcaaggattcttgtttgagataatcgccgccttgttgatgttcatcaacaacttcttggcacaagttgaatgcaaggcgttcgaaagactctccaattgggctgactttctcactgatgcagctgagaataacctcctctagatctttctggccatattccttggcttcagcaaaagcttgaagtagatgacaaatactcaattggttctcgattccctcctcttctctaggcaaatttagtggtgattggattgatgttgagtccattgatgatgcttcagaattcactattgaatttggactccacacatcatttccttcagcagaaaattggtgtgaagaagcttgctgtgatggaaaactcccttcactcccttccaatgaattaccatagaaaccctctatcatcaaattagtctcatatgcttccaaatccattgaaacttcctccacttgggatgtcacttgaataagatcatcacaaggaaacagggtggcaaaagggtctgatgcaatttccgaaattttgaaaacttcatttgagttgaacagagaagagaagtctgggctatcaattggagtatcatccatgttgatgaaatcaggttggtcaaaagatgagtctataaaattttcgaaaaaccaaggctcaagaaatgcagattccatcttctctctgatgaatagtaaatgttagaaagatgaagactttctgttggttttgatttgt

SEQ ID NO: 72 - DNA, 1014 bp, Rosa_hybrida_osiana
tagcagctttttaagccttttaacacattcttatatcttctctctcaagtctcatattatcctctgatctgagtacaaaaatgggattacaagctgctgcacattcatcatcttcttccatagacagcagcaaccatcctgaccttgatggtcttcttcctaagccttctggatcgtcttcttcttcgtctctctctcacaaaatcaatggtggttctaattctcgtaacagtcgcagaagcactcataccagaacagatttgagcactgatctcagacttggcctgagcatatcaccgtctcatcactctgacttgtcttccactacttcaagcccaagggaagaagcattggtaagctggccgccgatccaatcgatcttgaggagcacactttcaggcaaatcagagaattaccatcatcagaattcttctttatttgtgaaggtctacatggagggaatcccaattggtagaaaattgaatctgtttgctcacgatggttatgatgctttgatcacaactctgagcctcatgttcaagaccaccattctatgtcctgataataatcatgttcattcagagaaatattatgttttaacttatgaagatcaagaaggggattggatgctagttggagatgtgccttgggagatattcttgactactgtgaaaagactgaagattactagagcagacagatgttagctaaattatattgatttgttcatcttataattataattagactcatagttgtctgccctattaatccagttttgccttattggtggttcaaacttcaaacttcatactcgaaacactctgtacagcatcaatattataagagagattatacttgtttctctttctgggtttcttttcctccatcaaaaccaatcattgtagagtaggcaaggttgttaacatggggccatacaagggcttcattttcctttttccttctgcgttcttttcgcattgttaggtgaggtcgatgttgtcactacaagaa

SEQ ID NO: 73 - DNA, 1089 bp, Rosa_hybrida_osiana
gagagagagagagagagatcgaaagtttgttttcatgaaaatggtgcaagatcaacaggaaatcagaaagggcccatggactgaacaagaggacttccaactggtgtgctttgtgggcttgtttggagaccgacgatgggatttcatagccaaagtttcaggtttgaaggtggcgggagacaaataatagatggtcaagaattgctagaaagttaccagggaggactgataatgagattaagaattattggaggactcatatgaggaagaaggctcaagagaagaagagggctgtgtcaccatcatcatcatcttccaactgttcatcatcatcaaacatcaccaccaatgaaggaggagcaagcttttatgacaccggtggggccgaaatcgtggcttcatcagcagagaagaaaaatatcaacagtggagatcaagagaaagaagatggtaatgacactactaaaagggaccaaagggagtattcacttgatgatatatggaaagacatcaatttgccagaagtgaacagtattgaaccagtttatgatggcagctatggtgaagaagtctgcaaattttcatgccctttaatggctactcctcctccactgtcatgggattcactgtttaaaatagatgaagatcaagagagtaagaatatgtttctgcctactaccccaagtgacaatcaattcctgtcctgttttgaatatggaaaggcatcgtctctaactggctgattaggattctatttattaagatttgttcatatgtgaaatagtctactactctcccaaggaagctctaacctgtataataacaacatgggtatgaagatgtcttaagcttctctgagccagtggtggtctgcatcatgagctggagatcactagctagtttatgcattactgtagttcctgtgtcctcttgtaatcttatgtgttttcaattttcagtgaggaagagagaacttcaactttaccatgaatacttaattggtatctccattagtacaccatggaagtattgtatatatgtctttcagttaatttttgaatcaagctgctggtcatgcaactgtatcat

SEQ ID NO: 74 - DNA, 1283 bp, Rosa_hybrida_osiana
aaaaagttgttagatttttcaactttgaacatatctgaattcctttttattttatttttatgaaattttcattgttaaattgtgattagctagtatgatgtaagaacttgacataagaagttgaacattattaggattgaaatgaattcattttcacaatttgttgaagatccaaatgctctttgccaaacctatcttacaatgttttatcacatgattccaatcacagttcctaattgaacccgaattccaattcgttagtggttcaaaccattcatgttgttgtacgccggattcacaaataatggttggttaaaggtgctactatgattcatcacttggaatctcgccgagtctgcgctttggtatgggatttgatcaccaaactgaaacgtctggccatttagtcctgcattattggccagcatgttgtggagatcataagttggattgtcattcaaaagctgggaaattggtcccaagtaatccatgtccaaaaggctagtaatggaaaatgtccttgggaagttcagcacttgttcctctttgacatcattagcagctgtaaaatgctccatttggggacttgaatcttcaagttttggatcatccaagtaagccttgttgacatgcctcttcttatagatcctacacaggacccaatcatccaatctcatggacccaagttgcttgctggcctgtcttttcgaatcacttaaccgatactcatgcataatccaatccgtcttgaccccctttggtggtctacccttgtagaaaacaagagccttcttcacccccacatacttagacgcactgtgaattgccttgtctgtgccggtggccttccaataacctgacacggttgcgcgattgggccggactccgttcgggtacttccggtcccgggggctgaagaagtaccattcattttctccaaactctgctttctcaggcaattgccaaggatcaaacttgtagatatcgacttctgggatgatggaaacagggcacggctttgaaatggcttggtttttgaggtaatacacaatgagttcctcatcagttgggtggaacctaaaaccaggaggaagtccagagccattattggcctccattgttgtggtcggcaacaaagctggatcaaacctactgctagaagctaaacagatagttagggttttagagaaatgaagtgtttgaaatgtttgaacggagagggctcctccttttatggttgggggaatttgtcgttttgtagcgtcacaggggctgtgaatctg

SEQ ID NO: 75 - DNA, 2021 bp, Rosa_hybrida_osiana
tcatttagccttcaaactttgaaaaacaaaaacgccagtcacggccccttgtccgcgcgtagtagcagtactgtactaggcatagtaccacgattccacattgaaaaaacaatatgtttgattactcgttctctctgtattgctgtccctctctcctctccagtcgtcctcctctctttaaatccataaatcacacgcatacacacaaacccagtagtaggaagctttagtgttgcagaccccccccaaaagaaaaagaactccgaagccaaaaagacaaacgttttctcatcacaccaccaaaagcctccacctttcctcctctctctttctttcgtctctcaaaactcaaaaagctccaaccacacgaaccccaataagccacttccgggtcatcgggtccaacaccacccattttcaccactacctaaatccattgcctcctataaaataaaccccaacaccctctacttcgttttttggttctctcaaagactcaagaaccaagctttggtctctttttgttggttttctttcctcctaccaaagtcctcatctttacataaaagtccaactttttttttttgcactaatcaattatggcgggcaatccgcctgagggactcggagacgatttcttcgagcagatcatggccgttcctcaaacgtacagcggctccggctctgctgctgagtcagctggtggtggctatggagacgtggggtccatgcccatggtgctgcagctgggctccggaaccgggtcttcttctggctccggcgtcgggtacagaggaggagtcgggtcggttgggatgggtatgggcatgcctttgggtctgaatttggagcaggggtttcttggacaagacaggttcagagatgaagttgaacccaactctaacaccaacaacaacaacggtaacagttctaatttgatggcatggcaggaaagagattcggttcacatgacgagtttgtttccggcatttggacatttgcaaaaccactctgtccggccaacgccgccgcctcctcagcctcaccagtttcatagccaaccagcaccggggccagctgctgctgcccatcaccctcctgctatccgcccaagggtccgagcaagacgaggtcaagcaacagatccccacagtattgctgagcggttacgccgggaaagaattgcagaaagaatgaaggctttgcaggaattggtacctagttgtaacaagacagatagggcagctatgcttgacgaaatcgtcgattatgtgaaatttttaaggctccaagtaaaggttttgagcatgagtagacttggaggagcaggtgcagtggctcagcttgtagctgatgtacccttatcagcagttgagggagaggggattgaaggaggaaccaataatcaacaagcatgggagaagtggtcaaatgatggcacagagcaacaggtagctaagctcatggaagaagatgtaggagctgccatgcaataccttcaatccaaggcactttgcatcatgcccatatcactcgctcctgcaattttccggacacatcagccggacgccacgacaatggttaagccggaatcacacaactcttcctaggctcccccaaagttttagttagtaccattcttactcatggcattgggtatattcatatatactctagtcaattacaactcattagaactgaagtgacaatcaagtcatagcaactgttcactgtttttcccagttgtgtaaaacaagggactgattgtcattttcaagtctagggaacaaatttcaaaagctaagttgatgagttcctcttgtaatatcaatggtggggttagttgagtgtgaccaaaatgttggtggtaagaactaagaactcttcaattttcttctatggataatgggatagggggccatgtaaaagggatggatggttgtgtatgatgtattttgagaaaattcttaatgcaagttgaaggtagatgataatggttggttgttttataaagcagtgtt

SEQ ID NO: 76 - DNA, 1334 bp, Rosa_hybrida_osiana
caatactgaatgccattcttgagccttgcatactggccctaattctttgtccacatatatatatgaaattctaagtacgtactagtactaatattaattaattacagcgcaagagagtactgaagccatacttaggtgcatataattctactattcatggcttctccttgtgactaaatgaggaagctaaatcttgcaacatattatagtcatgaggaacatgtagctgcggctgctgctgttgattctggtgaagagtactcatgagatttgagtataacatggaacttgctggatcatgatgatgatgagcttgcagttgaagttggttgttattgactggagtaagcagttgagttaacaataattcgtgtggaaatctcggggattcgttgttactagtcccgaatcctcccgatgcagctgatgccaaaaaggaaggcgtcaacatgccaacagcattgatactgcctcgaagtgtggctggacattgatggttgtgctgcccttcatatgttgtgatcacaatcgttggatcttggaacgatctctccacacgtttctttacaaggcacttctgagtagtgcatctataataacttctaggataagggctattcttgactgccttctgtccgtactttctccatctgtatccatcttcaaggtgatcaatttcgctcttggtcaagaaggcgaaccgtggttccctttgccgtttctctttcttttttgccttgttcactttctttgacttgtcttcatgatctccaggagcttcttcagactgtactacctttggatgcttatctttcttgctgatcttatctgaatcttgatcgtttccatgctcatgattagccgcaccagcttcattagatgaacaagataacgatgaattcggtgtggatgggttttgatctcctacaccagcagctacagccttggtattcgaattatcgtcctgatctaacagcggcggagaaatgacttcagatgatgaacatgaaatgtcaaaggcttttgagagggtggtgtagtccactgacccatttaagcagtcggcgaagctcatcaaatatgaaggatcggattcaaaaccatgtaggttttgagtttgggggtctgctggtgtttggggaatattgtatatggaagaattagagccataattgaagaatggaaagcttgacctgtcgatttcgtgggggttgtaatcgaaagggtcatactgcaggtaagggcttttcttttcttttgacattgaagatgatacagaggaagaagagaaagaataaaggagcaggaagaggactttgagagagatggggtta

SEQ ID NO: 77 - DNA, 1128 bp, Rosa_hybrida_osiana
aaggcatctgtactgaatgctcagtttttttttttctttttggttgttaaaatgcagaaattacaatcagtccatatatagactctctattgggtatacatcactagatgagcataccacaacttggccttcatctatctcatgcttctaaataaaccttttctctcatctacccattcagaaagtatccttccaaaaaaaacaatgacagaatttctattaacctcagttcgaaatccagataaagctctattgtcatcctacccattccctaccataagtcaactgtgtgatacagcaatgcaactaattcaactcctaccaataagcaacattggatctttctgttccattacatgctgaaaagactgatatacatatatacaatacaaattgttatgtgtgtgggcgtgggcgtgcgtgtgtctaccattttcaaaaactggttcattaattgtagataacatataatctcaaattgcaaataagatgctaccatagataaggatagcatttattcacataccaaaggctgatgtccataagccgccatcattcctgcataatatggatcctggtaaggatttgaggcacaagcaatagagtgaccaatcagttcaagctgtggtggctgagcaacgtcttcatcatgcactgtaggaacagtggacgaaacaagctgcaaatcctggtttttatttccaactgactgaggagatgtcatttgtgactttttggtggcattatcatcatcctcatttgatccatcatcagaaacagatttgtcatcactggattctgaacgactctttgggcgttcaaaagaagatgaattggatgcatttatccctgccacggctggggaggtggggttatacccaacagtacgccaccaaggttctgaagaaacagtggatggtggaatggtatgacgatcagcaggtattcggttttcagtatcagactttggctgcatgcctcaaatatccgaagttttgccgggagagagagaagccaattaaaccctagattttgggcgtcacggaatctcacaatctctctgcaattccatacaatctcactgtgtaggtaactgtactggaagacctctcctcctccttctcctct

SEQ ID NO: 78 - DNA, 2116 bp, Rosa_hybrida_osiana
caaatgcctcttcactatcctagaaggcagaaaaaacctagctcttccttgactcgagagagaagctctccctcttcttaggcttgagagagatatggcggccgccttgagttggcttactgtctttctatacccgaaaaaggactcaaaccctaaaactcaagatggtgagcatgtcaaggtctccaagattgtcctcagcctatagttcaactgctgcaggattccaaatgcctcttcactatcctaggtacacgaaggctgactacgagaagatggaggagtggaggttggatctgcttctcaagcaatatggcctaactgctgtcagcaatggaactctcgaggagaagagggcttatgcaatcggagctttcttgtggcctgatcagtattaagagacgtactaccctatataaataattcctctagctatataaatcgcatacatatctcatatgtgtgtgtatagagctagcctggtttgtaatgtctaattattcgattttggtctgtaatttgtgtcgtatgcgtacgtagttgcattccagctagctagctagtacctgatcgatgcagttgcatgtcgtttgcaaacaaggagaaagttaaagccagaaatccagaataatggttattgatagtaagttgcggaagtaaaatatatggggcatggcttccggtacctgacttgataaatctcaatgaatgtcaaataattatttagtagtagttacatgtattcactatcttactgtttatttccttgattaatttttttctttttttcttttcttcttgggaattcttttcgttaattactttgtctggacaaatccacgtacgtatcacggtatatattatcgaaacattcgaatttcgaaaccataattaatctgctgttctatgaactgcttgtgttagttccaggtgctgatccttcaagattgcagttttcttctaatttttggtaacgaaggagatcaaaaatgccagaagctttgatcatacctttgtacacgatcaaataaatgagaaccactgatcttcgtcacactttaaaaaaaaaaaaaaaattaaaaagtaaaaaaaatttgaggggagaggcacgtgtcaacagatctgagcttgtggtcccacacctcctcctcccacacggcggtgccgaaacacaagcgagcactcgggcaactcgcccagatcggcgaatagcgactcgtcctcttcgcccatcgggaacaccatccccgccacgtcatccgtgtcggccccaccgccgctctcagcgaaaatggggctctccagcacccgcgacgacgtcgtttccatgtccgcgaaccacccgaactcgtccacgacgctcgggatgatcaccagcgacgactcctcgtcgccgccgcccagatccgccgcgaacagcctctcctccgggtcgggttccggctggccggccgccgtctcctcctcccgggtctcggctttgatcccgggtttcgtgccgtggtggcttctagaagccggccaggggtggttgtgctctgacgagtaggtgatcaccagcgtggtggggtccacgcggcttctctccacctgcttcctcgctggacacccctttgagctactgcacctgtaataccctctggggtaaggtgagcccttgatgggtttctggccgtactttctccaggcccatgaatccgacggcgggggagtgtttgtgttactgttttccttgatagggattgacaccactctcttctgtacagcccgccggctacttttcttgggagacgaagcgatcttggagtagacgacgtcgttgaaggcgggaggaggggagccggagctgttttccggggtggagctcgtggtggtgttgttgtaatcgtcgtgatcaatatcactcatgaagtgtttattgaagaatgtgggggagcccatttctgaaagctagaagtgggaacttgatggttttgatgatgaggcttgtggaagacacaaagtgcagaaagagagagggagatggagaagggttgggtggtttttgatggggaggaagaaacaaatggagggaggggagggaggtgtagaaatcgggaggtgggtccttaaaaaa

SEQ ID NO: 79 - DNA, 1027 bp, Rosa_hybrida_osiana
atttttacccctctaaggactcatgttatcactttgaggactaatgttaccactttgaggacaaatatggtaaactaagaaaatttaccactttaaggactcatgttaccactttgaggactaatattactattttgaggactcattttaccactttaaggcaattgtatgcatgtcatatgttaacacattgtagaatttctctggtagagaaaaccggatagcttttcaatagttacatcttctacttattaggtttggtatattactaattttaaaattgaagttctgtgattttcagtcttatagcaatctggttttccttttggctcatatttggatgtcaattctgttcagcactgtggttcaaattttgaattttatgtttagtttatttttattttttttttgtttgaagccaatgacgttggaacaagttttgtccgatcaagatagtgaggatgaggttgatgatgatgttgctgattttgaagatcgcaggatgcttgatgatttcgttgatgtaaccaaagatgagaagcaaatgatgcatatgtggaactccttcgtgagaaagcaacaggtgttggctgatggccacattccttgggcgtgtgaggcattttcaagattgcatgcacatgagcttgtgcaatccccggccttgatatggtgttggagattatttatgatcaagttgtggaatcatggtctcctggatgcacgcagtatgaacaactgtaacattattcttgaacaatatccgagcaaggttttggatccgaagagttgaaatagattagtttattaaagtccggcaaattcatacaaatgtgttgctgatcctagtgacactattaacagtatgttttataatgtgcttattattcccaagtttaggcgggaggccattttgtcttttctgtaacgttttgtatcccattttccgtatgtaatgagctgtatattaatatttacatcacagaaaagatatgtaaaccagcagttgacaaaattcatttgtgtt

SEQ ID NO: 80 - DNA, 2417 bp, Rosa_hybrida_osiana
attagaggcttcatagaatgttgagtgacttgggtcacacaaaagcaaccatttctcagtttacattacagtaagcaatagacttggtgaaagggaaaatgtataagacgctgtatcatcaagactgcaagatatagaaatggtactgattaggtgcttagagatcgactcccatgagcttgataaataaggcagtggcatatcaattggatacaacataataagtaagactttagtccgttgagttaattgtatacaaaatttttatatctctcattattgcttggacaatcagcatacaacattacaatgagagagattgataaatcatgtatacaactgatgagaaggaaaatcacaccctacgatatgcacctttatgatgtgttttttgatcgaatagaaaagttgcagtaaaattaagaggcagtagcccatggagggtaaaagtcgatagtggacatgctccaaaagctacaaaagcaaacctaacacttgacaacatgaaataacacacaaaggcccaaaaaatgggccaaaaacatacgttcagcccacatacgttcatctgactcaagaaccaaactccgccaagatttgccgtatacagcagtgagaagcagcagccaaactcggtgagaatgattccaaagttgaaatcacagctccaaatgactcatggacaccttcaaaaacccaatattgtcaatatatatcgtgtttcaagaaagaaaaaacaaaactaacagttctaaggtgtaaaatttcagggattttgctttacttcaataatattttgacttttatgttttttttttcttttttaagaaatttcagaatctgttgctcactaatatactgttaacattcccccataaagttaaattatgttcatgctaactttatacattcatggtaatctaaattgaatattttgtgatccagtgcagtatgaaacttctagtttgtaaatgggcacttagatgctagatcaggttcctgggtttattgcatagactagcataccaactacaaagttcatcctagtgcatatatcggtgtgcgctaatcttttggctaagaggacaaattgtacataggcccgttgtatttccacattcgatcccatcacatctagtctaacatgtgatcggaagagtattattgcaacggttatatacaaaaatttctccttgccaaaggttagctagctctttgaaagctttatactcttttgcctccccattgtacttgttggcagaatcatcctctccgtgacatgatggtgctcactgtcactgtttgtatacaactatatgtaactttctcacgcaactagagagacagacacctagttgtatagaccggtgacagaggggagttacttgtagcgatagtgtccacaagtggcagcaagaacatttttcttatttttacacattttcatctttcaagaccaaaaatcccaccactggtaatcactagtcgattggtcaaagagaccaccatcggagttcaatctaccccaatcttcaggagaagttaaggacccatctgcagattccacaaagttcacaaggttcggttcctcctcaagtccaaagtactctgcctttatgctgctatcatcatctgacataactcgaagccgatgttctgatttttccaatgacatctggtaattaggcttcccctcagattccgacctagtagcatcatctccattgtccgattcgccatcaatgctattgttcctttcctcttttggcctttgcatcttatcattcagcttctgcaactgagtaaccaaggcctgcttttctttcttgagggcttcaaaccgggaagccaagttattgtagttggctctgagtatgctgtagtctctttcaagctgcttcgacttccatcgagctctcttattctgaaaccatatcgcaacctgccttggttgcaaccccagctctttcgccagctgtagcttcttccttggctccaacctcgactcggactcaaaaatggactccaacgatcgaatctgctcatcactgaacctcttcttgttgttcttgctgctcttcttctttaaactactgcctgcacctaacgagtcgttcatgtagctaaaagcctcggcttcttccggtgagggagaatatccaactcggtctacctctaacatctcgttgtttttggggatcagtgtttcgggagagaatgcttttgggcttttgtgctttaagctatgtgatcctcagtgtatgtcatggttctttgggtctgactgggttttgtgattgctgctttatatccaaaatccatgggtggatctgctggcgtaatatatatatatattgcttttctttctgttcttttaatataaacattccccatactatttttcttgctcgaatatt

SEQ ID NO: 81 - DNA, 1323 bp, Rosa_hybrida_osiana
agagagagagagagagaattctggagttttggagttcctctgtccatcatagctagatagatggggaggagtccttgttgtgcaaaagaggggttaaatagaggagcatggacagctatggaggacagaattcttagagagtatataacaactcatggtgaaggcaaatggagaaaccttcccaaaagagcagggcttaagagatgtggaaagagttgcagattacgatggttgaactatctaagacctgatatcaagagaggaaacatcactcgggatgaagaagagctcattatccggcttcataagctcttgggaaacagatggtctttgatagcgggaaggcttcctggccgaacagacaatgaaatcaagaattactggaacacaaatattgggaagaaagttcaagatcactcatccactaattctgaagccaatattactcaccgcaaaccaccaaatcatcaaacccagcaaaagaacacaaacgtcgtccgtaccaaggcatcaaggtgcaccaaagtgttcattccccacctgtcacaaatggatgaagagactaatcctactgttcaacaagttgctgcacctttcttgaatcatgactattatgaccaggtcaacaataatgatgatccaccgctgaggatgatggggactggtactgatcaccaaccagctgatgatttgcccccattccttaatttggagattgatgatgaaaacagtaattcttgcgggtttatggtagattttaagatggacgagagctttctttcggagtttctcaacgtcgatttttctcagctctacagtagtactagtactgctaatggaggagatggtgctaaagctgctattagtaccagttgtggagacgataatcatcataagctgcatggcccagatttccaatcatccatggctcctatcctcgactctgaactggactggctctcataatcgagtagatatactcgcagctaattagtataatgtatatatatcagctagaaattagtttcttccacataaccttatcagtcgcgaaatgcaatgttaactataatggacatccgcattgcacacattacaaaaataatgcatgcatgtcgattggtcgattgatcagtgtcaatttgacatgtcgatccgcacttcgttttctatatgtgatgcaataaaggtagtcaatgtgatcgatcaagggatgaaattatttgaataaaaagtctacttgagtagatgatcgattgatgggggaatattttatatatgtccagaataatccctcagaatttaatcagtgtacataagtct

SEQ ID NO: 82 - DNA, 718 bp, Rosa_hybrida_osiana
ttctccttctgcttttccatcatgcggtttgggatgatagcaccctgctgtactcctatcccgcatacacattgcttctctccatattagacatgatttgcatctggtagaaatttagtgaactcatcaagcaaatctggctggtcactaaataaagcagcaacctcaaggtaaacctcagttataggcttgtcctcgtttcggtacttgttcagtatatctaggaatgccttataaacacggttgtcatcatctcggaaacgttcctttatcttgtttacaaagtcgatagcttctcgaaactcagctggcttctttggaggcacatcttcatcgaggcttatctcaataccctctggcaagaaagtattgaaacccataattaagttgttatgcccttcaaatagttcctttactttggcaatgacatctactgtgaaaattctatgagttttgaaatctctcatgacctcaagaaacctttcatatatgtcaattcggtcggaaaacacttccttcactgctttgagatacgctacagcatcttcaccagtcaattttgatgcgactcgtcttccagttactcctccagatatttggggtggcccatttgagtcagcagatgaagaaccagaaggcgctttcatttcagaatctacgggaacatcgtctggtattcccgccatccctgccaaacaaaacccaacaacattaattt

SEQ ID NO: 83 - DNA, 1732 bp, Rosa_hybrida_osiana
gatagagaaagaagatgcccattttcatttgatagatacaattgtgcagtagttattagatatctttggaatgtcaggcaaggcaatataggctacatcaatagggttgtcagttttcatccgagtctttattgctataggactggaaaggccttctgcttttagcctctgtgtgtttcaaaggcctgagatgggttcttgctgttacatcctcgattctctgaaggaatttaaggctattacagctatgctgtaccttatgtttagccttggctttgaatttctcctctgggagtcatttcataaaaggctcaggcttttctgtcaagtgaagaacaagtgaaacagtggtgtggttaatggttgaatagagaaggacagaggagactcaatggcagcatcaacttcttgtgccagtaagagcccatttcccatgaaagacacaacctctaacttaattccatacccagcacctctagctaaatatgaggaggttgcagcgaatcctaagctgttcatgtctactttggaaaagctccatacttcaatgggaaccaagtttatgatccctataataggaggaagagagttagacttgcatcgactgtttgtggaggtaacttcccgtggtggtatggcaaagattgttagagatagaagatggaaggaagttaccgctgtattcaacttcccctccacagctacaaatgcttcttttgtgctgcggaaatattataattcgttgcttctccactatgaacaaatatactatttcaaagctcaagcatggaatcctctctcttctgatatttcacagaaccctaacatgacttccggtccagctccaaggggaggaactaaacgaaaatcaactgaagctcagacactactgcttcagcaaccacagaaaattccagagattcctggagccgggacaccagcaccgtcagaagatgacactgtgttagggtttatagatgggaaattcgaaagcggatatcttgttaccgtcaccaaaggcacagagaagttaagtggtgtactgtaccaagttccacagaatccagtacaggaggttccacaaggcccacagaattatagcatggtggtcaacagaaacgacagtacctcagctgtacctgttccccatcgtcgtcgacgaaggaagaaatctgagataaaaaggagggaccctgctcacccaaaaccgaacagaagtgggtataactttttctttgctgaacagcatgcaaggctgaagccacttcatccggggaaggatagagagataagtagaatgattggtgaattgtggaataaattgaaggagtctgataaaggagtgtatcaggagaaagctgtgaaagataaagagagataccgagtagaaatggaggaataccgggagaggttgaaggctgctcaagtcatcagtgatgcagtcccactacagcagcggtctcctgaagcagacgcaagtctggcagaatcagaaaccaagattgaagaaaccgagcctggcgactctcccgaaactgcagatgaaagtagcgagagtagttctagcaaatcagagaagtgatcgccaaggtgatattaccgcaaagaatgatgtgaatgtagaggcatctataggagccaagtattgttctgaagttgtatgctgatgagggtttcatctcaccacattggagaggatatccagtggtgaaaagaagcctgaggtagtaat

SEQ ID NO: 84 - DNA, 1372 bp, Rosa_hybrida_osiana
tgctttcactgataagtgcttctcccttctttaaacagatcagggagaaagtatcaactaattaagctcgatcatggcaaagaacataaaaagttcttgcaacccaggaatactgtatagatgttaaaatttgactagaacatagatatatacacacacacgtataagaagcgaaatattttctctacttctcatgatcattcgctactggaacacaaacagtacgtgtgatagcttatttgggagcacacataaaagaatgattagcctttgatgaactaaggttaaaaatataaaaccgaaagaacccagaaatcaagaatagcacggaggggaccaagtataagttgtaattaactgatttattttagatcaaatcaccccagaaacagagggctgatagtagaggccttgggattcccaagtgccgttagcagcagctgcccaattgttgtcacagtgcatatattgctgatctgcagaccaagcaccacctcccattgctccttcgatctgtgacagctgctgcgtcacgcctacggcggtcactttaccatgatctctacttgttgttggcaactccatcgaacactgatctgtatatgacatactcacaattgactgatcagttggtaccggccatgtaggcataggaagtgtgttggaccaagtcacttcatatgctggaggttcattggtccacaccatgcccgagttcgatgcaacgtgaccggacacggccacctctgtctggtcactgatatggaataccacgtcgtggcctgtcacgtgatcatttttgtgagagggaggttgtatacaccgagtgtgactaaattttttttcctttgaggaccttgctatatatgaaccgtcctgattcggagggtttgcaaatccaaaaccaccgccacgtggcacaactagaggagacgaatcaatcttctcttgcacccgactaaatcccttgccatcaagcaacttggccttgagcgcaccaagcaagccatcttcttcagctccattgccacaaacatcctcaaacgaaaacggttccacattttccatccccgaatctcccccgcccgcaccattcgattgaggcagttcgaaattggtgcgggcatttgcgccgcgcaaagtccgcgcagcgtcatcataagccctagcagcatcctcagcagtatcgaaagtgccaagccaaagcctgaccttttgcaacgagtctttgatctcggccacccatcttcccgacggcctctgcctcactcccacaaatctatggtgccctctcgacgatgacttcctccggccgcggacgccatcgttttgagctcgtacagctgtcatggttagctctagcttagcttga

SEQ ID NO: 85 - DNA, 604 bp, Rosa_hybrida_osiana
caaaccaaacacattgtgcatgccttccttaatttcttacacaatcataaactagctggctcagctcgcttcactagtgaccactaagcaagccttcaatggcatctgaaatgacaccacctgcagaactcgaacttccgggattccgattccatcccacagaggaagaacttctcgagttctacctcaagagcgtggtgttcggaaagcgattgcgcttcgatataatcgagtttctgaacatctaccgtcatgatccttgggacttgcctggattgtcaaagattggagaaagggagtggtactttttcgtgcccagggacaggaagcatgggagtggaggaaggcctaacagaaccactgaaactgggttttggaaagcgactggttccgaccgcaaaattgtgagcttatctgatccgaagaggattatcgggttgaggaagactctggttttctacaaagggagagcaccaagaggaaccaagactgattgggtcatgaatgagtatcgtttgcctgataactgcaaattgctcaaggtaaatattgatgcatcccaatttaatttttctgtagtatgtactactagccattaacgtcg

SEQ ID NO: 86 - DNA, 1133 bp, Rosa_hybrida_osiana
gcgttccggtccgggaggggatgattacggaggttagcggcgtggctctagggcggcggtctggctcagggatgggtattggcggctgctgcttggatcggagcggagttaccagatctgggtccgttcttcgtcgatcgtgggtgggagctggtttggtcttccggcgtgttgcgatttgccggcagggttcgatttgctggttgttgagggtgtctcggctttggacggtggagtggcagtggccggattctagatctcttggaggtgtcgagcccagtgtggctccgattcttctcgggtttcgatctgggatgccggccatcttctggtgtggttggctttgtcttgtgtcggtgcggtggcgttgtgtcggtgcggtggctttgtcttctcaaaataggaagaaagcagttaaaggtccttgtgaagaaggtacaagctgctcgtgatgaaatacaatggggtgaagagggccctcctccattgctcgtgaaaattgctccagatttgtctaaagaagatctcgaagatattgctgcagtggcccttgcccttcgcttggacggattggtggttacgataaagaagacaaagcagcaagagcctacgatctagcagctctcaagtactggggtccgaccaccaccacaaactttccggttaccaactatgagaaggaattggcggggatgaagaacatgtctaggcaggaatttgttgcttcccttagaaggaaaagtagtggatttgctagaggagcctcgatttacagaggggtgacaaggcaccatcaacatggtagatggcaggcaagaataggaagggttgctggcaacaaagatctctacctaggcactttcaatttctagttgaatacggctgctggtaccatgtctccctttgaacatggcgagttttttgttcttgatgatggtggagaggtaattgttccctgtcatgtttcttcaaactgtatcaaaggtagtggatgaagatattggagatgatgaaaccaagttttgttcctctatagtcatctaagatcacataggatatcaacatgtaataaggaattatttagttggttttaattgtagcttagttaactattgtcatctaggattagataa

SEQ ID NO: 87 - DNA, 1068 bp, Rosa_hybrida_osiana
aaaatgtcaccgtcagttcattcatacaattggctgcagaataatcaaacaacgtacaaacatagttttacataccataaacattgggaatatttgtacaaataataagttctcggctaagattttgtcatccaagcgcattggcaagttactcacactactagccacgtcttccaatatattctacacccatgtaacaaagacatacatacaaataccatgcaaagcttcttaaaatacttcgaccacattcatttattacaaatgccatgctaaaaatacttccaccttctggatctccgcagtttagccacggtaaggaagcccaagtttgagagataaggtgtcgtcagcagagtcgacatctggggaagaaccagtagcgcagcagctgctagcatttgtggcagattccgatgataaaccttcttctgccgttgagatatccgactccaaggcgagtacaccagctttgtttccatttgcgttggatatcatccccatcgtctgccttaaatgattgttcgcttccaacaactcagctcccttggtgtgaagcacacggctaagtcctccttcaatgtcctcctccaatttcttcaactcatccatattcagcccttccagatcctcaccattcatctgccttagcacgcgggtcttgtctgcaagttccttactcaacttgatgcgatcaagctggagctcaagagatggctccaacttctccacattttcaatgtgcgctttgtaccttgcaataacatcctttgtgctggagctggaggactcgaagagctttccagtagcagagaagatgatgacagcatactcacaatcacagagaaccgacagctctccagctttcttcaaaagccctcttctcctcttcgaaaacgtcacctgcctcgccggcaagttgtcgatcttcctgatctttatcttctccctcgtcggcttcgacatagcttagctagccagcagcttgctttcttgggcgttgaggaacctgaagttgaaggaggtgaagggcgatactagtttgctagctgaacaggagtggaaaat

SEQ ID NO: 88 - DNA, 848 bp, Rosa_hybrida_osiana
gtgcaaggagaagtgggagaacataaacaagtactacagaagattaaaagatagcaacaagaaaaggcccgaggattcaaagacatgtgcttattataatgtgcttgatgctttgtacaacaagaagactagtagggctgagaatcaagttgattctaattatgaattgaggcctgaggagctattgatgcatatgatgagtgggcaagaagagcagccacagccggaatcagtgactcagaatggtgatagtgacaatgttgagaagattcaagaagataatggaaatggagagggagtgggagatgtagctggagatggctatcggattgtagctgctaatccttctccaatggaacaagataatccttcagtggcaattatgagctgaggttaagtgatttatgaaagcttaagttggtgtgtcttgtttttgaagtgagtcggtttccaatttttggttaccacatggttgttgttcagatcgtagttttgagaactgttgagaagtttattttgttttagtgcagtgtaaaatgggatcgtagaaagaaatctatactttcatcgtttgttgaatagaagatggtagagactagggagagaataaaggagggatattgtagttggctgttctgtggcttttggatggtttattccctgtttgtaaaagtatgaatctcatacatgaaatttatcgttctttaactgttcagctaatcacagtgtattatggttaatttgcttgttgagtaaagttcgttgtttaccctttcactgtaatgctggctgtgtaaaagggaggaattggagtatctttatttttttttacttttttttcttgtgtt

SEQ ID NO: 89 - DNA, 1677 bp, Rosa_hybrida_osiana
tttttcttcaacaaacccctctcaatatctcagttcatagttttttattctctcttccttttataatagattagctctataacttacccgatctgacaaaatcctagagcttgtagccttgtatagttgataggtctgcacacaatagtcttcggtagaattagacccctttgcagtttatagttccataggattaacttggttttctgttttgatttctggaatttagagatttcagattcagaaaattttcttaggataacagtgtgtcaagcagaagaaatggaaaatgtttctgcgtttattaatgaagatgagcagatggaattgcctcctggattccgatttcacccgacagatgaagaactcataagccactacctttccccgaaggttcttgacagtggcttcaccgctagagctattggggaggtggatttgaacaagtgtgagccttgggatttgccttggagggctaaaatgggtgaaaaggaatggtactttttctgtgtgagggacagaaaatacccaactggtttaagaactaaccgggcaactgaagctgggtactggaaagccacaggcaaagacaaggagatttacaaggccaaagcccttgttggaatgaagaagactctggttttctacaaaggaagggctccaaagggtgaaaagaccaactgggtcatgcatgagtacagattggaaggcaaatactcagcctataatctccccaaaacagctaagaatgagtgggtgatttgcagaattttccaaaagtgtagtggtgggaagaaaactcatatttcggggttggtgagggagagccctttcggaaacgaattccgcccttctttcctgccaccactaatggattctccaccctacaacaacagtgacacaagaaccaccaccgtcggtgaaacatcgcatgtgtcctgcttctccgatgcaatggaggatcagaaaacccaggatgacattatcgacagcttcaacaacaacaacaatatcagcagcagcagcaacaacaacaacaacagtcatctgttagcttcttcatcctcatttccgttcaagccctcggtgcagaactcttacttctccgaccaaatcgcaccaaacaccggaaatatgcagtacccggactctggttttatgcaagaccagtctattatgaggttgttgactgagcctcaagctccaagcttgaggcgcagctcatcaaattatttggggggtcaagatgttccatcatactcagctgctccaatagagtttgattccatctggaattactgaactcaaagatgtgacggtcttagattttctggaaaaatatctaagatcatttaccgtaccaaactattgtgtacttgtatagattaattagatcctcctcgtggagtcatagaagcaccaaggatattagtctgtatgtgaagaagaaaaccataagttgttttacttgaacaaacatcaaatggaatgtagtctcgtgtgttgttatagtgtaaactcaattcgttattggtttgataattcttgtgatttggtagatacagtggcaaaggtgacatatatgatttgtgttctttctcttgaagttcagtcgttaggtcaccaaccgagagttcctcgttgatcaataattagtggcatactacatgacgtcca

SEQ ID NO: 90 - DNA, 2267 bp, Rosa_hybrida_osiana
aagttttttttttttttttttttataaaaaaagacaagcccttttgctcctccttgttcatattcaaacctggttaaggtttctctttctccatctccattctccgagaggtaaggtgagtggtctgagctttgcatattagatttgtggacccaacagatttgtgactttgtgagctcaaacttgaagccattccgggttgaagaaggttgtgcttcgctcaattggacccaagattgacaatagcagctccttaggcttctgttaattatcaatccatatcaagcttcacgcagcgtaacttttggttgctctagggagagagatacaccgattattccaggcataatacaaatgtaacttattggagcataacacaattgttggaaatggacattaaggaggcagagcgggtagtgatagctaaacctgttgcttcaaggcctacttgttccagctttaagtcattcacggagcttctcacaggtgctatcgatgcatcaccctctaatgtatcttctgaaactgctgttcctgccatcagaccaaagactgtaaggttcaagccaacagcgactcatccggttgttggattggtttctccccaggcagagacccctggtgctgcacttagtaattcggcagagaaggtttcaaaatcagataataaatcaactgtggtatataaaccattggcaaaagttgtatccagggcgactgttactgccttggctaatctgggaaacttcaatactagccagcaacaaccacaatcatcagttggggttggagtggttctacattcaaaccgtgaaaaatgctttaaaacccaacttatctccaacctctaccagaaaagtccatcatgtgctgagacatgccagagtacagaaccagtaaagatagtaccacaaaatatggaagaggatgcaaaaaatataccagctgcagccaatagtgataggccttcttatgatggatataattggagaaaatatggacaaaagcaagtcaaaggaagtgactatccgcgaagttattacaagtgcacacatccaaattgtcctgtgaaaaagaaggttgagagatctttggatgggcagatcgctgagattgtatacaagggagaacacaaccactcaaagcctcagcctccaaagcgcagctcctctgggacgcaagggtcaggatttgcatctgatgcaactggtcaagataacaacacccggttatggaatagtcatcttaatgaaaagaatgaaggttctgaaggtagagtagaggatcagaatgaagttggagtacctgtgcattcttatcaaagcaaatccatagtacattatgatccacttgcaactggagcattaaatgctggaggtgcaactcctgataattcttgtggagtcagcggagaatgcgaggaagcaagcaagggagttgaagcagaggattatgagcctagaagtaagagaagaaaaagtgagaatcaatcaaatgaagtaggcatattgggggaagtcatgcaagaacctcgtgttgtggtgcagagttctgcagattctgagattacaggtgatggcttccgctggaggaaatatgggcagaaggttgtaaaagggaatccatatcccagaagttactacagatgcaccagtgtcaaatgcagtgtgcgtaagcatgtggaaagagtctcggaggatccgaaagcctttatcacaacatacgagggaaagcacaaccatgacatgccactgaggaccgcaaacccaggagcatcatcagaaaaggatacacagcccccccctacaagtaaagaaaagccatgaatctgtcaaattcactctcagggatcaagctgttgaacccccatctttaatctgacaaacttgtcaaacaccaaataagttgcagtgatattctgatgcccacttttattggcctgctgcaaatactttggtggaatggaatctctttgaaagttgccgcttgatggagatatttatttatgtgcgtttctggaaacatagaaatgggtgaaaatattttggcttaattttccctattgccggtgctgccttagttcattagtagggtagcatattttttttttttcctattttaattgttattaattgagccacttcatagttcatgtaataaaaatgtcttagttacacattaatggcagttaggttgataacttggtagtgctcatcccactaaattgtaaggacaaggacacgtttaaagattcaatttttagtgag

SEQ ID NO: 91 - DNA, 2249 bp, Rosa_hybrida_osiana
catttgctcgcagccaaacaaccaaaaccgccgcccccccccccaaggccattagctcacattagagagataacattttttcagtctttgcagagccagtagcccaaagactcgagcttttatgagctgtgagttcagagtttgttgctaaaacgtgaaattttggtaacattttacagcattattaactttggtgtagtttgagcgacactacatacctgtctcaccgactcagcatttcatttcctcagttgcaaaatccctgcaagtacaagatctctgtctctctcagattatgtaccttagaagatggccttccacctgataagattgtcttccagctaagaagattatgctttatatgtctccttgtacagtttacttggtgtgctggatttgacttggtcgtttcacctccaagtactttaactgtggaaatgtagcttgtttctgctgattctttgagtggattttgaatttaaatgaaactcaaagtctgctagaatagtgttcagtgttgtgcaaccatatctcacttttgatcgtctcacttattcctttattctaattcattgtctcccaatctagatatatacatccctacctgtagatttcctccacttgattgtggcacatttcaccacctatctgatacatcgatgaaggtagggctttcacatgtaatcatgagtcaccatggagtcagttctgtatcacaaagtcagactaccaaaggaatcacagagtcatactgtacatccctatccccagtacatgattttttgggttgtgaatcagaaggcaggagttcagtggcccgtgaatgttcatcagcacgcctctcacctttcatacggacagaatcttttagctctcctactaatgtgcgagaatctagccttcaacattccaagagcacattttcacgttcttctgttttttgtacaagtttgtatcagtcatcttcatcgagttctgaacggcagcttggaaatctgccatttcttccccaccctccaacatacagccagtccatttctgctgttgattcaaaatatccaatgcttttaagtgaggatgtgagcaatcaatatgatgatgaacaatcagattatctcatgaaggactttcttaatttgactggagatgcttctgataatagcttccatggaattggccgtggaggtgacactatagcacttacagatcaattggagtttcagtttttgtcagatcaacttgacatagctatcacagacaatggagaaaatccccggcttgatgaaatatatgaaattcctcaagcctcatcgaaaccagccatagaattaacatgtagtaagagttgtggctcgacagcaccacctgttgatgctctttcaagtcacccctctcctgggccttcatctgcacatagaccaagaatgcgatggacgcctgagctccatgagcgttttgtagaggctgtaaagaagcttgatggggctgaaaaggctactccaaaaggcgttttaaaggttatgaatgttgagggcttaaccatctatcatgtgaaaagccacttacagaagtaccgtcttgccaagtatatgcccgagaaaaaggaagataagaaggcctctagctctgaagaaaagaaagcagctgcaagcggaatggaaagcgatggacgaagaaaagggagcttccatatcactgaggctctccgcatgcaaatggaagttcagaaacaactgcatgagcagcttgaggttcaaaggtctcttcagctacgcatagaggagcatgcaaagtacttggagaagatcttggaggaacaacagaaagccggtagtgcgttactttctccacaagccttgtcgtcactgactactaattccattaaagaccctgaacagcatccttccccatcagctggtgtatcaccttcacaaccaactgagtctgactcattatcacctctgtcactgaagcacaaagctgctgactgtagtgactctgaggcacaaggatgcactaaaaagcttcacattgaagaaaatccagatgagcctgtagttgaaaatctctcggcaacagtaactactactactgcttcccaatagctgcactaaaattcattatttgtagtgtaatgtatagaacttcagggcttttgtaagtaaagttgtaatcaagacgtgctgctttgtatcttttaacagttggtagttcaatgaaaaagaaaatcggctattctagctaagttc

SEQ ID NO: 92 - DNA, 2920 bp, Rosa_hybrida_osiana
gaatatgctttaggtaatgacagaaatcacaagaccatgtaggagaataagtagaacctttaaccttttagaattaaggctgacatttaagcaacttctaaaagtactctaggtacactagagatgttcatccaactaaggaaaggtagctacaatttgcaagagcaataaccgaaatacattttccttcttctagccttagagtaaattacttgaaaaaactagtaaaactgactagacaaacttagtctccaccaacagaatcaagttctatgattttggggtcacctagggattctactctcttttacaagttacttactgcactttcaacgaacgctgctaagctagactctcaactgtatcaaattccaactccagaacctgttgattctatggcaaagagaaactccggaacaattacaggcctagttggctccctgagtcattagagtggctttctctttctggagccttcctcttgcgatatgtatcgcttggcagccctaattgcaatgtagtgtcagaatctccattgtcaaatgcgaaattgctgaccaaatcggggcttttggcaccatggtacgcaggggagttctgtttttctacaggacaatagtcgagataagatggaactgcatgatcagcttggggaaataaacgccgaagctcctcaacctgtttgcgcaaggtttcattctcatgtatagcacgctgttcctttactcttgattgctctagttgctccatcagtaatttgtcctttttctccttcactgaaaataatccttcagttaattgattttctagtttctgcaattcttccaagctcaaactagacaagtcattgcccaacagacgcaattgattctgttgtagcttctcaagttcatcttttagaacatccacgtccttagggtcatcctcctctgcccttgatcctaccagagcggtctctgaagactcataacacttgctgtatcttgcaatagttcgcttcatactgtaaaaaattggtacagataaacatcagcagctataatttgtataaacagggtacaattaatttcatgtggctatagcacttgcattgcagcatcatttctaagttattatatatggaatcagtattagatcagcaaccgaaataatgcttcgtctcaagcttaatgcaatcatacaagagatgctctaaccgaaaatatatggtagaaagataccaatggaaccaactaattaactaaagacatcgctttgtttcactaatccttggagaaaagaaagtatggttgccaatgaaacgtcccataaagactggggactggagaaagcacctttacctaaagaggagacagcatcttcagagaaagactaatagcttaagagagtagggatactcaaatattgtaatatcagtgaggactaaggacatatgaactctcactcaagtgttaattgaccttctataacaggtggctacacacaagtcaagacagtcaaacagagattgtagacttatttatgcaaggttaataggttagcagagattcgagacttggttaataggtcattagagagctcaacaacaaaattcacagtctggatataaaatacaggaaccagccagaccggcattgcatcagcataacgcgaagacttaaagtcatgtaagaatcctcaaaatcaacccatgtccaacttcaccaaaggcacaaacgaggaacaaccaaacaagggagttctcacgaaacaaaaatctcctttaaagtctagtagagtagcttttttttctgctataggtttgaacgctagagtgaccagattaccagtaatggaagctgaaactgaaccttccaaagtaaacaaaaccttttacagatacgcatcaacattagcttaccaaggtccacttcagagtcaattaagatagaaaagaggtagccattgatttaacaacaacaacaaggtgaccagatttcaattctggactcgctgatccaacttgtcacaaataacacattgttttgcagaatacacagcttcaaattgctcattaatcatcactacccacagaggaaaacccccaaactcaacaactattagtactaaacaactattgagcagcaatgtagggattgacattcacacgaacccaaaagaaaacaagggaaacagaagcacccactagtccatataatagaataaaaacaataaaatataaacaaattatagttgtgatgcagacaatgaaaaatttaaatcaaagccccagataacttcaaccaaagacaagaggaaaattataataaatagagtaaacatgatcagaaacacatgcatgaacctcaaaaagttcagaaaatttagcccaaaattaaaaaaaaaaagtaggaaaaaatttacccagcactggaaaactcaaaaagcttgccagtgttagagaagacgatgacagcaacctcagcatcgcagagaatagccaattcctgagctttcttgagtaatccagcacgcctctttgagaatgtgacttgtctgctgttggtgttctcaatcttcttgatctcaatcttccccctacccatttttttcttcataacccccaaaaaaacccagaaagccaaaaccaattaataaccccccaaaaaaaagcctaaaggctaaaggccagagctttagactacccaaaagcctaaaggccaaagctttagactacccaattcataagaaatgaagaatctttctgggttttgtctctgcctccctctccctctctctaaaatcaagaaaaggagtggtctttctgggtctctctctatctctcttgttgggaaagaggtgggttgccaaaattagattggggcagaagataaaggggcaaatagtgtgtctgtgtgtttgagagagagagagagagaga

SEQ ID NO: 93 - DNA, 1520 bp, Rosa_hybrida_osiana
gaatatgctttaggtaatgacagaaatcacaagaccatgtaggagaataagtagaacctttaaccttttagaattaaggctgacatttaagcaacttctaaaagtactctaggtacactagagatgttcatccaactaaggaaaggtagctacaatttgcaagagcaataaccgaaatacattttccttcttctagccttagagtaaattacttgaaaaaactagtaaaactgactagacaaacttagtctccaccaacagaatcaagttctatgattttggggtcacctagggattctactctcttttacaagttacttactgcactttcaacgaacgctgctaagctagactctcaactgtatcaaattccaactccagaacctgttgattctatggcaaagagaaactccggaacaattacaggcctagttggctccctgagtcattagagtggctttctctttctggagccttcctcttgcgatatgtatcgcttggcagccctaattgcaatgtagtgtcagaatctccattgtcaaatgcgaaattgctgaccaaatcggggcttttggcaccatggtacgcaggggagttctgtttttctacaggacaatagtcgagataagatggaactgcatgatcagcttggggaaataaacgccgaagctcctcaacctgtttgcgcaaggtttcattctcatgtatagcacgctgttcctttactcttgattgctctagttgctccatcagtaatttgtcctttttctccttcactgaaaataatccttcagttaattgattttctagtttctgcaattcttccaagctcaaactagacaagtcattgcccaacagacgcaattgattctgttgtagcttctcaagttcatcttttagaacatccacgtccttagggtcatcctcctctgcccttgatcctaccagagcggtctctgaagactcataacacttgctgtatcttgcaatagttcgcttcataccagcactggaaaactcaaaaagcttgccagtgttagagaagacgatgacagcaacctcagcatcgcagagaatagccaattcctgagctttcttgagtaatccagcacgcctctttgagaatgtgacttgtctgctgttggtgttctcaatcttcttgatctcaatcttccccctacccatttttttcttcataacccccaaaaaaacccagaaagccaaaaccaattaataaccccccaaaaaaaagcctaaaggctaaaggccagagctttagactacccaaaagcctaaaggccaaagctttagactacccaattcataagaaatgaagaatctttctgggttttgtctctgcctccctctccctctctctaaaatcaagaaaaggagtggtctttctgggtctctctctatctctcttgttgggaaagaggtgggttgccaaaattagattggggcagaagataaaggggcaaatagtgtgtctgtgtgtttgagagagagagagagagaga

SEQ ID NO: 94 - DNA, 2409 bp, Rosa_hybrida_osiana
gcaggatttgggctttgaatcaagtaatgaagtaataaataacagaaaagcacaaatatatatattctattacaagcatagacttcttacacagttaccccaatgttagatagtcctaagtccttatttatttaactcggaatacatttatcagtataagttttgttcccatacaaaattacaagtcaagtctgaatgaaagcatagcagtgcttggagcttaatgataaatgcacattcaccatcacctccatgctgaagcaacaatcaagcttttgtcttcccatccaaaatgaagtccacccatttcttcctttatcttgtacctgtcacaatacattctgataaggtcacgaatcgaatcagtaacacttttactcataggagttgaagtaaacccagccatggccatcctggctctccacttccctgcaacctcataccgctctattctttcctctccttcgcatgcaacgatgtttactatgtcccgtgcaaggcactgcttctcaacattcatcctatcctggctctcccttggtatagctgcttcaagggaatcaaaaacagcagagtaataattgtaggcttcaacaaatcttgggaagaatggggtagtattggtgttcacatcttgttccacaactgtgacgagttttggactcaagctcttggccagccggagaagctggtctctctggtttaccgtcgaaacactctcatcgggcatgtggtgaagctgaaaagcaaaatttatgacgagtgcttccccttgcctacagtgcagcatcggaggactgatgattgaagtctttgaggcaactgcttgaaactcgaatgaaacaccaagtccttctgctagctgctctagcctttgcccgatggtattaaggcccccaacaggacgctgaactgactcagggtcatcaacccccgttaacctcaagtgtggtggcttaccaggcagttcagcaagtgcttgtatcagtgttatgtactgattcccctggtttatgtcaaaatctattatatggacactcttttcatctttcgtagcttctataatggctccatttgctgccatgaatccaaatttaaagcaagggcacacctcaaaaagtacttgcatggctgcaaggcgataagaggatggaggttccttgcatttcaaagctctatagagatattttcctgaggaagccatgcgagctacaaggccttccaccatatatgctgcaatcctctgaggaggatctccctgaattgagaccagctgccgaagctcatttattatggttgatgcttcttcgatatttccctctgaaagtgcaccagcacagtcaaacagcagctgcttgggtgttcgaggagatactcgtgatatttctttgttgctgctgatgctgcttagataagaatcagatgatgaagactccttgggtgagtcatggagcaatgcattctggattggttcagtccattcaccgtcaatttccatgctttgaccatttccaatcatctcctcatcatcttcagcatcgttgtcctcaagcagtgctctctccaattcttgaagcttcaatctcatcttaccgtcatcaaagctaacagcctccgggctttgactctctaagtaatctgagtcaatgttggtttggtaagcatcatgaagcctcatggacatgaaagaagcactagatggattttgagtggtcaaggaggaaacagatccagctctgcgtggataaaatgaggcagcttgctcattaaacgaacttccagaaatgctagagctagatggatgcacaacttcttcacttggggagtctaggaactcataactctcggtggagtaagaatcagtcgtatacatggtattattcttatcagcaccaaatgtttgggtgttagtgtcattgcttcccttcagagagtagagtttggattttctgtacgacgtggcagacagctccactggcctgactaaagacatggttttcttttacttcttcagttgttgaggaagactttatattatagaatttagatctgatgctgaaatatgaagaggatcaaagaccatgtgcgcaagcagtgatgatgtccttatggaaaaaagcagtgagaaaccgagttccacccaacaattcccttcaaggaaacttcttctcttttcttcagattctcagttttgttcagtctgcatgtttataccaacttgtcttttgactaaggttgttgacagaacaaatgtgtcgctccccccattatagtgtttcttactttggtcagtcaatcctgactgtacaatctgcattccaatgactgaacagacggtggcagattgggtcgtgcagaaaaagtcgagggagttggggaaaaagtactacttgaccggtgctggtcatc

SEQ ID NO: 95 - DNA, 2364 bp, Rosa_hybrida_osiana; identical to SEQ ID NO: 47

SEQ ID NO: 96 - DNA, 3415 bp, Rosa_hybrida_osiana
tatattacactagtaaagtaaactttacttcagatcatggtgaattactgcgatcgagttattaatacatcatcaactccacctaaagcacatcagtggccacatgaactggccgcgcttgcttgagtttgaatagatagtaagttcaaaatatataattataattatatatatatatatcaggcttggattacaggtcaatcaagccggccgagaccacctcaaccatgcagcagagtctagctagtgacaagtctttacgacacggtggtgttcatccctatcccttgcaaagcagcagcgctgttgagaagcttcattccctcttcactcatctgctgaacttctgttggcgaaaggattctgatgcagcgcacacaccccacaaattcctcccaaggatcatccccgacaagtagaacatcgttctcatagtccacataaactagtttccaccctgaacctcttgggtcgttaagcagcccttccagtccaaacatgcactcaattgcagaacatagttcctcatagtttgtgtaactagtaacatcaattgatctaccaacagatcccgccttttgaaccttggtgtaagttcgcacgggtgggaccacttggtgccagctattctgcagtagactgctctcatcaagatcaacattgcttgaggatgtaccgcctgagttgtctgccagttcttgcctggagaaagcgtgagaatcccctaggcttgcagacgtaatctgggactgcagatcctgactcgagctcaagttgccaaccaaacattcggaaggattctggaagtcggcatgctttagtttagagaactcatccattatggtgcttgtaacagaagggtcagcaacaatattactcacaccagtacaaacatcaacagaggggcaaccataaaccccactttggttgttgctctcatcggacaaatctctcagactccccgagttgggaatagaattaaaggtagatgggtcttgatgggatgaagtgagctgatcaacttgcggtaaaagcctactgaggctattatcccacatttcctgacccattgaagttggggagaaattatttgcttcagttaaaacagatggacagtcctgcaacccatatgcagacaaaggccctggtgatctcagtatcccagcaaagggttggtagaacatgcattcatcattgtctaaggatggaagtgaaccatttgcactattaaaatctgactgaggtagatcagtttgttgggagtggtagagtaatgattccataggttgctgcataggccgtgggctagcttgtaattggccctggttctggttgacgcatgcattgttataagggctaccagcaatcccagaagccaatttttcctcattgccctgtcctgaagagctcaactggcttaaccgatcagttgaaagatcaggttctaacttcgttttgtcagtgttaactccaactggtgtctgacttccaaacttagctgtggtgttcaactgggaagggttttgactctgcagagacataccttcagaacagtaaaccatattcttttggttcattttcgcctgcatggcctgcatatcttctagtgcacctccattagcagccgattcctgttgtagagcagacaatgttccagcctggctaacttgaggttttagtagcatattaactagctgttctgaacatagatttgacatcggataaggaaaagagttcatgtttccaatctcaggaacccggataaaaggtcttttaatcatattggcccactcagtttctgcacccaagtatccagagtgaaatggacgtttaagactcgaagtcagggaaggaaatatgaaaagactttcaggagtctcaacttcccatgagctgaccctattctgtttatcgcaacaccccggctcgtcccactctacctgaagatttcgccacttggaaccaggccacctcagtggatcgaaatcactgatattaactattgtacccatgtatctacgcttgccagactcctctgtctcaaacatcattccaaacctcatgccaacagacagttgggtcccatatagagccttttggaatttgaccaaaggtataacaaattctgaagggcatgcccttggattgtagaatatagtgaatggacttcgattagcagcagcatgagctgcagcagcaaggacaccgatgtgcatgctatcagcagatagaactgaggatggtaatgtggtctgctgacgatttgctcgccttacgccaaccaatagctgtgacttctcatccctgataaatagcacagaatcgccagctctaagcctctttgtgccaacaaacaaactccaaccagttgtgagaaggtggcgcttcggttggccgcggtaaatgtggcgaaatgtccaggtattatcatgcaagtctcggacaacaagttcttgggttggaggttgcattgtgaaatccaagggaggaaagagcttttctgctgccctccgcggcacagagaaaccaccatgtgtgcttgtatcacttgcagtcaaaattttgcagaaaaactcacttggatgcttgctaggcttcagtccaaagtctggtacagggaaaacatctttttcagaattgactggtttaagacacatttgtgtgaagatctcatcggtttctttgtctgcgtgtagcgtaacattttgaacttggcatagcaactgagacgggagattcggatagttgggaatttgcgaggttgccattctctttgtggatactgccacctgctcgctgtggccctgagggaagtaataagcaagactccccacttgaggcaaacaaacaagggggccggcgcaggcatgccataactcggaatttatggccttccgggacccagaatgatcctgcagttcttttaacaacttcatctcctcaagtatactagactgtgctccactaagcaaccctcctccaattctcatcttctcttccattatgacgtgttctttgtggttggttttttgtttggatgcctttcatacttaagctggtaatagtctagccagttttggatccgatctcgctgtctcacaagtctggaaaatttgtttgcattgtagactgcctggtgacaaagatactcattcgggtggtttcttttgaagtattggtccacagtgtctgatattgaacgaccagagacatatggtatgtttctaaccactaccgtgaactgttctgcacgcctgcgctgtgaagctaagaatttcaacctcattgatgctacatagttatactccttgtataacatgaaacaagtccagaatgtgaacaagtattccaatcctatgtggtaaaaaaaccttatcgacttggggcgcacatttgatatagagagcctatcaat

SEQ ID NO: 97 - DNA, 3540 bp, Rosa_hybrida_osiana
tatattacactagtaaagtaaactttacttcagatcatggtgaattactgcgatcgagttattaatacatcatcaactccacctaaagcacatcagtggccacatgaactggccgcgcttgcttgagtttgaatagatagtaagttcaaaatatataattataattatatatatatatatcaggcttggattacaggtcaatcaagccggccgagaccacctcaaccatgcagcagagtctagctagtgacaagtctttacgacacggtggtgttcatccctatcccttgcaaagcagcagcgctgttgagaagcttcattccctcttcactcatctgctgaacttctgttggcgaaaggattctgatgcagcgcacacaccccacaaattcctcccaaggatcatccccgacaagtagaacatcgttctcatagtccacataaactagtttccaccctgaacctcttgggtcgttaagcagcccttccagtccaaacatgcactcaattgcagaacatagttcctcatagtttgtgtaactagtaacatcaattgatctaccaacagatcccgccttttgaaccttggtgtaagttcgcacgggtgggaccacttggtgccagctattctgcagtagactgctctcatcaagatcaacattgcttgaggatgtaccgcctgagttgtctgccagttcttgcctggagaaagcgtgagaatcccctaggcttgcagacgtaatctgggactgcagatcctgactcgagctcaagttgccaaccaaacattcggaaggattctggaagtcggcatgctttagtttagagaactcatccattatggtgcttgtaacagaagggtcagcaacaatattactcacaccagtacaaacatcaacagaggggcaaccataaaccccactttggttgttgctctcatcggacaaatctctcagactccccgagttgggaatagaattaaaggtagatgggtcttgatgggatgaagtgagctgatcaacttgcggtaaaagcctactgaggctattatcccacatttcctgacccattgaagttggggagaaattatttgcttcagttaaaacagatggacagtcctgcaacccatatgcagacaaaggccctggtgatctcagtatcccagcaaagggttggtagaacatgcattcatcattgtctaaggatggaagtgaaccatttgcactattaaaatctgactgaggtagatcagtttgttgggagtggtagagtaatgattccataggttgctgcataggccgtgggctagcttgtaattggccctggttctggttgacgcatgcattgttataagggctaccagcaatcccagaagccaatttttcctcattgccctgtcctgaagagctcaactggcttaaccgatcagttgaaagatcaggttctaacttcgttttgtcagtgttaactccaactggtgtctgacttccaaacttagctgtggtgttcaactgggaagggttttgactctgcagagacataccttcagaacagtaaaccatattcttttggttcattttcgcctgcatggcctgcatatcttctagtgcacctccattagcagccgattcctgttgtagagcagacaatgttccagcctggctaacttgaggttttagtagcatattaactagctgttctgaacatagatttgacatcggataaggaaaagagttcatgtttccaatctcaggaacccggataaaaggtcttttaatcatattggcccactcagtttctgcacccaagtatccagagtgaaatggacgtttaagactcgaagtcagggaaggaaatatgaaaagactttcaggagtctcaacttcccatgagctgaccctattctgtttatcgcaacaccccggctcgtcccactctacctgaagatttcgccacttggaaccaggccacctcagtggatcgaaatcactgatattaactattgtacccatgtatctacgcttgccagactcctctgtctcaaacatcattccaaacctcatgccaacagacagttgggtcccatatagagccttttggaatttgaccaaaggtataacaaattctgaagggcatgcccttggattgtagaatatagtgaatggacttcgattagcagcagcatgagctgcagcagcaaggacaccgatgtgcatgctatcagcagatagaactgaggatggtaatgtggtctgctgacgatttgctcgccttacgccaaccaatagctgtgacttctcatccctgataaatagcacagaatcgccagctctaagcctctttgtgccaacaaacaaactccaaccagttgtgagaaggtggcgcttcggttggccgcggtaaatgtggcgaaatgtccaggtattatcatgcaagtctcggacaacaagttcttgggttggaggttgcattgtgaaatccaagggaggaaagagcttttctgctgccctccgcggcacagagaaaccaccatgtgtgcttgtatcacttgcagtcaaaattttgcagaaaaactcacttggatgcttgctaggcttcagtccaaagtctggtacagggaaaacatctttttcagaattgactggtttaagacacatttgtgtgaagatctcatcggtttctttgtctgcgtgtagcgtaacattttgaacttggcatagcaactgagacgggagattcggatagttgggaatttgcgaggttgccattctctttgtggatactgccacctgctcgctgtggccctgagggaagtaataagcaagactccccacttgaggcaaacaaacaagggggccggcgcaggcatgccataactcggaatttatggccttccgggacccagaatgatcctgcagttcttttaacaacttcatctcctcaagtatactagactgtgctccactaagcaaccctcctccaattctcatcttctcttccattatgacgtgttctgaaccaaaagaactcacctttcacatcttcaatgcaaaacacagaacccaaataaaaaaaaggcggtcacttgcgaccaagaaacctcttattatttttttccccctagaaactccaaaacctcctctgactgacccagctcagcttgtctcatattccttcaaattttacaactgaccaatttctgagaatcctataaaacaaacaatccaatccaatccaaatctaacaacacaatgcagcagggggaggacacaaaaagcagaagcccttagagtccaaccaggaccaaagaacaaaacctgcacaaaggctctttttttcccaagtagggttgcttcaccttcagctttggctctcctctggctttggctctgcccatcagtgatttgcatcaaaggcacgcggaccaaagcatgagttctcaatcgagtacttgcttttactcttgaaaagaaccagacctttcgatgaaccccccccctttttttttttttttgtgttctgagattttctgtttatcgg

SEQ ID NO: 98 - DNA, 1981 bp, Rosa_hybrida_osiana
ttcacgttcagcttcagaagcgtgagcattacactctctttcagtcaacgagcccctcttctctatcaattacaggctgccactactatcatctttcttagcaaatcccagcaatacaagcagattcatgatatgaatccttgttcgattttcaattcacttctagttttacatctctcactttcttccatttgtgtcatccatggcactcgggaccaaaactacggagaagagattctaaacgcagcccagaaagacaaaaactggttggtctcaataagaaggcaaatccatgaaaacccagaactcagattccaagagcacaacaccagtgctgttctacgcagagagctggaccaacttggcatctcttactcgtacccaattgccaaaactggtattgtggcccaaattgggtccggttctgccccagttgttgctcttcgggctgacatggacgccctccctatgagcttgctgattgggagcacaagagtaagattgatgggaaaatgcacggatgtggtcatgatgctcacaccactatgcttctaggcgctgctaagttgcttagtcaacggaaagagaaacttcagggtactgttagacttatttttcaacctgctgaggagggaggtgctggagcagccgagatgataaaagagggtgctcttggtgaagcggaagcaatatttgggatgcacgtcgattatatgatacccacaggaagcatagcatccatagcagggccacaattggctgctgttagcttctttgaagcaaaaataatcggtgaaggtgggcatgctgcagctccccatatcactactgacccaatactcgctgcctcttttgcaatttcagcattgcagcagctcatctcaagagaaactgatcccctccagagtcaagttctgtctgtcacttatgttcgaggagggacggcatccaatgtaattccagcctatgttgaatttgggggcacattgaggagtcttacaacggaaggcttgtaccaactcatgaagcgattgaaagaggttattgaagggcaggcagctgtgcatagatgtattgcatacgttgacatgaaaagtgaagagtttcccccataccctgctgttttcaatgacgaaagcttgaattcgcatgtaaagagggttggaaaacttgtgcttggtcctgagaatgtgaaggtcggcaaaaaagtgatggcaggtgaagactttgctttctatcaagaatcgattcctggaatcatgatcggcatcggaataagaaatgaggaagtgggttcagtttactcaccgcactccccttacttctttcttgatgaggacgtccttcccgtcggggcagcattacatactgccttggctgagatttacttgaacgagcaccaacattccgctgtttagttcagagcatagttctagagaaatattgatgattcctattgctttaattgggtgctatcttatgtagtgctctgtaggcaggaaaaggaaatgtaaatttttttttgttaaacaaactatatagatagatacattatcatttagtgttcttcatttcctcgcgttgcttccagtagtagtgcttcgcaagaatacgtagagtaagtaggagtagatctggctggagtagctcagctgctaccttcaaaagttcctcaagaagcataatctgttgttctcctgatgtgtacctttccattgcttcaaaaagggatttgacgagtgttgtatctctaccaaaagcagataatagtgcatctgtttcataagcattagcgaacgccaagctttcacgccaatctttccttggcttggatggcagcatcaccatcagtaccaactcacactttagtccacaatcaatgagaaactcattgagaattgtctttatttgatcttggccttcagtgcttttcagaagcaagttgctctcatccacacatgaagacatgcatgtcttcatg

SEQ ID NO: 99 - DNA, 1621 bp, Rosa_hybrida_osiana
gagatagactacatagactagtgcttctttaaatgcatggctgcaacaatacatacgtaaaagtctggcaaacttaccaaccgagtctactagtatagtaatgggaaagaaacagcttgaagatcaaactactagctagaacttgaaaaatatataagaataacatataacaaaacaaataaatgctctcaataatatattattatatatatatatatatatatagatcgaaaagaacctcaaaattaaatagcaatacttatgcagtactactaccactagctcggattgcgcctattccagttcttgttatcatgctgatcttcctgaccatgatcgtcttgctgtccttgttgttgatgagacggataatatgttgatggtggaaactgtggagtagaaaactgcggatgattgaatggattggaagtgtactgataattcgagctgaaatcaatatcattgctttgaagcagctgcgcatattgatatagatcaggaaaactactactttcctgctgctgctgggatgctgcaaccaagggagcatcatgatgaggaacggttgcagctggtcggccaagatttcgttcagtggagacactgctgccgctacttgttgtgccggatgaaccctgcataaggaagcttgagttgccttgaactcgctcaggaaagttgagcttggcttttgtgcctttgaacttgagagctgcattatcatatgcaatggctgcatcctcagctgtctcgaaagttcccagccaaactcgagctgctttcttagggtctcggatttcggctgcccatttgccccaaggcctttgcctcactcctctataatgtctttttctcacagtactctcttgatcttgaaccggttgagaccggtccggttcatctttcactgaagggtttgattgcaccttgttatcttcttcggggtttccgataacttgagtgagagcagaaaccatggctgacatgtcggcttctgtccacgaagattcgtagctagaaggagagaactcatcgtgcctctctttctcctccgacgtttccgaaggaagtggccgctttccatgcctccggtgatgatccaccttttcgagctcgaggtactacttggcaaagatggtatataaagatggtctagatttgtaatatcctgcttggtgtgaagaagattaggacatatatcaagatttcaagaccaaggatctatcagatccgagagtatataagcgttgtggggagaggtggtaatgaaaagaaagcacaagggggagagagagggagagagagagagagagacgaagcggcctcttggctgaggattgaagaacgaggtcgcgaggcagcaacttaccaagcttgaagaatccgaaatagaaaagttgaaaactgaaacctgaaatctacttgagccttgaagacgtctatgctttgctattgaaaattgaatacccagaagcagaaagtaagagaaacaacaatcaattgaatacccataaacccataaatcgattgaagagtgaaaaacaaaaactcagaccaccgtcgcactgtcagtcaatcatggcgtcaccgtcggagagagaaaaggcgtccgtgcggccat

SEQ ID NO: 100 - DNA, 1635 bp, Rosa_hybrida_osiana
gagatagactacatagactagtgcttctttaaatgcatggctgcaacaatacatacgtaaaagtctggcaaacttaccaaccgagtctactagtatagtaatgggaaagaaacagcttgaagatcaaactactagctagaacttgaaaaatatataagaataacatataacaaaacaaataaatgctctcaataatatattattatatatatatatatatatatagatcgaaaagaacctcaaaattaaatagcaatacttatgcagtactactaccactagctagctactactcgctcggattgcgcctattccagttcttgttatcatgctgatcttcctgaccatgatcgtcttgctgtccttgttgttgatgagacggataatatgttgatggtggaaactgtggagtagaaaactgcggatgattgaatggattggaagtgtactgataattcgagctgaaatcaatatcattgctttgaagcagctgcgcatattgatatagatcaggaaaactactactttcctgctgctgctgggatgctgcaaccaagggagcatcatgatgaggaacggttgcagctggtcggccaagatttcgttcagtggagacactgctgccgctacttgttgtgccggatgaaccctgcataaggaagcttgagttgccttgaactcgctcaggaaagttgagcttggcttttgtgcctttgaacttgagagctgcattatcatatgcaatggctgcatcctcagctgtctcgaaagttcccagccaaactcgagctgctttcttagggtctcggatttcggctgcccatttgccccaaggcctttgcctcactcctctataatgtctttttctcacagtactctcttgatcttgaaccggttgagaccggtccggttcatctttcactgaagggtttgattgcaccttgttatcttcttcggggtttccgataacttgagtgagagcagaaaccatggctgacatgtcggcttctgtccacgaagattcgtagctagaaggagagaactcatcgtgcctctctttctcctccgacgtttccgaaggaagtggccgctttccatgcctccggtgatgatccaccttttcgagctcgaggtactacttggcaaagatggtatataaagatggtctagatttgtaatatcctgcttggtgtgaagaagattaggacatatatcaagatttcaagaccaaggatctatcagatccgagagtatataagcgttgtggggagaggtggtaatgaaaagaaagcacaagggggagagagagggagagagagagagagagacgaagcggcctcttggctgaggattgaagaacgaggtcgcgaggcagcaacttaccaagcttgaagaatccgaaatagaaaagttgaaaactgaaacctgaaatctacttgagccttgaagacgtctatgctttgctattgaaaattgaatacccagaagcagaaagtaagagaaacaacaatcaattgaatacccataaacccataaatcgattgaagagtgaaaaacaaaaactcagaccaccgtcgcactgtcagtcaatcatggcgtcaccgtcggagagagaaaaggcgtccgtgcggccat

SEQ ID NO: 101 - DNA, 1458 bp, Rosa_hybrida_osiana
tagatacaataagctctctttgcctctacagaaagtcttaacacacttacaaataggaaaacaacactgtttcaaatctacgtacccacaactattttctaaagttccaaacagttacttgctttactgcgtcaacaacagggggtgagtggaagtggttcatgactgaagacagcatggaatccatcacctatctcctgtggtatcgtcccaataattacaaagtaaccaaagccaagaatgaattgcctgacagacattgcacaagaagtgtttttccaagctggagactgcacaccttcagcaacacctcatgggggaattttgtcatttactgtgaatggccccacgagaggatccattcaactgcattgtttccttctgttgaccaaggaaaacaccttccgtgccgtcattaaatgttgtacgatatgttgatgatggggcatggccattgccattaccgttggagagtgtgtggttattgagtgtcctgaaccatgaacctgatccttcttgctggacattggaggaaacgaaattgccgttgctatcattaggaaagcactcaaaaccagatttggcagatttgggtgaaaagtttgcatccaaattaatggttttttctgatgaggaatttgcatcactatcatcttgcttcttcgtattaaggaaacggcctccacaaccccttgctctcctcaacgcatgctggtgacgagactcatgaagatatggcttcctaacttttataagcttcctttcaagttcagctttagcacgtgactgtcttcgtctcaaaataccatggtattgcttggcatttacataaacaggctcctcttccatttcaaagggcaagggcattctaccatgatgcaagccatagaattgagggggcaccatagcttgtggccgataatgagtcaacatcccactgtattgtggatctgaatatggatatgatgtcaaaacaattgagtgaccaacaagttccatttgtgaattcgcatcaagatgttcacccaatttagcaaccactgcagaggaactgcttttcatttgttggttttcatgtccactattgccatcagactgtgatcctcttgtagtctgcacatcttttttgaagttagctccagcatcagcttgtgactgcatagcgccattcatcacaacacggttaccatgttccccaaaagacaaactatttcccgctccacgccaccaaggtagtatagttgattgcaacacagtctgggttcctggatctacatctttagatttggctggcatcattcaccactttctcagtggtgtgcgaagtaagggatagaaactccggaagagctgcggtggtttttagggattcgaagtcgcggaggttggactcattctttctcgctcgttctctctctctctttctttgcaaccttaagagagacagtcgagaaagaa

SEQ ID NO: 102 - DNA, 864 bp, Rosa_hybrida_osiana
tagagagagagagagagagtttaactaaagaaaccaatacaactgattaataaatagaatctgtaagggacgtacattttatttttggttcaaaactagctagctagctattacacagtaatttacaaagcacagcaggcatgtaattgtcactgcaaagtactgctattatatatgaatcagaaagaagtgtagatttgcatctgggtcaaaatatgctcaaagttatcggtagacttctcaatgggatgagagtgcatgccttcatatgtagtcaccacaactccttcatccttggttagtctctgaacctgctttttcacattgcagccctgatgcgtgcaccgatagtagcttcttgggaatttgttgttcttgactgctttttggccatactttctccacctgtatccatcgtcaagtatatcgacttggctcctggtctgaaacgcatatctcggcttcctgatcttcttctctcccttcttcatccctgcccttactgctgtattactttcaccctccccaaagcttttcatgctctgggaaatactactcatgctgttgatactgttcgaaacctccatctctgacatcagccccaagaacccatttgacttattgttgctagcttggtactgatcgttactataagcatgatgaggatgactgttcaccatgttcaatgacagagaggaagcagcagcaggtggtgtcgttgatgaagaagagtaaaatgttgggtaggtttccatcataaaacagagaggtagagaaagagagaggagtccaatagctataagaaaagttgggaagattgattcaagatggtagtgtgtggtttatatataaaagctaaagtta

SEQ ID NO: 103 - DNA, 2596 bp, Rosa_hybrida_osiana
gaaatgttccctcacacgtcctatcacctcactctgttttcttctctttcttctttcttctctcatctctcatctctcttctctgcctctctgtgttgcctatgtgactctacgactcgaatcgtacagagcaattagtactcgccggcgattttaaacccatcgccgagactaaatcacttaatgcgatcaaactcattagcttaattcagcgccggcttccactaccactgctccggccacctgattttgatttctcctgcgatcgccaaaacgcaccgtttagcgtttcgttctgttgttttctcgcgtgttcggattgtaatttgagctccgatcatgtttttgacttagtggtgtgtgatcctccttgtcggtcgatctccgccgttgatcgacggtgagagataggggaattgagctaggggagtgaaatcggagtcatgttggatcttaatctcagcgtgctgggatcgtcgtcggaacaaaacgacgtcttcgtcgtcggctcgtttccggaaggctccggcagcgcgacgcaaatggacgagtccgggacgtccaactcgtccgtcgtcaatgccgacgcctccagcaccaacgacgactcgtgctccgcacgcgccgccgtgacgacgttcaacttcgatattctcaaggtcggtggaggagaagaagacgacgtcgctgtgacgaaggagctgtttccggtgaacagttggccggggaaggggcaggggcagcagtactcgccggcatcatcgtcggcgaggcacaacttgatcgagcttggtgggcccgcggaggttcagcagaaacagccacaaccaccgccaccgccaccacaacaaccgcaggtgaagaagagcaggagagggcccaggtctcggagttctcagtacagaggggtcactttctatagaagaactggtagatgggaatcgcatatttgggattgtggcaaacaagtgtatttgggtggatttgacactgctcatgctgctgctagagcctacgatcgagctgcgattaagttcaggggagttgatgctgatatcaattacaatctcagtgattatgaggaggatatgaaacagatgaagaatttgaccaaggaagaattcgtgcacatactacgtcggcagagcactggtttctcgagggggagctcaagatatagaggggttacactgcacaaatgtggtcgttgggaagctcgaatggggcaattcctcggcaaaaagtatatatatcttgggctattcgacagtgaagtagaagctgcaaggtcctaatgatcatgaattacactctacctgacactgaatttattaccctaaactctcaacctcattgggttccatccaacacttctctcccttttgatttttctggaattagggcttatgacaaggcagctatcaaatgtaatggaagggaagctgtcaccaactttgaggcaagtacttacgaaggggagatgatatctgacgctgttaatgaagatggcaatcacaatcttgatctgaatttgggaatatctcctccttcatttggtaatggtcagaagcaaggcgaggggcatcttcagttccattctggcccttatgaggggcaaaatggaaagaatatgaggatggtgcccaatgcaaatgcaacaatgactgctccacctttcagaggcgtaataatgacatcagagcaccctcctttatggaatggtgtatatcctagtttccttccgaatcaggaaagagcaatagagaagagaattgcattaggatctcaaggacctcccaattgggcttggcaaatgcatggccaggtcagtgccccaatgccactgttctctactgcagcatcatcaggattctcatttacagctaccactccgtccgctgctgtcctccccataatcccctcaaacccaaccgccctcaatctctgttttacatcttcagccacggccgcccaccatacgtctcaataaaaccattaccacattgaagcggtggtagccgattgcctccatatattcaaagggcatccacaacaaactggccggtggtccttcagattccttgtatgtgattcaaatagagattgatacatacactagtacaccacaactgactccatgtttatcaaccacactctgaactctatttgtatttgttctctgaaatatgttatagctgacctgagctctaatgtaaagtttcgagttctcatggtggcgctcgtattctaagacggttaccgatttgaatgtttgtttgtttaatgtaaaccgtataatgctttccagcatatgggacagaagtatttgtacctgtcccaagtttagaaaagccatgtagaggaataattatagttgctatatactctgtccaaaacaaaaggatgtacaagttcacagcttcatgatgcaatgggacaatttttagtgatgggtgctgtagatattgctgctttactagtggaagaagaacaaagcttggaaaaaggaggtcgtttcccagttgtcttgcgctgaaaagcaattttctaagttctaaggaatatcaaaaccaaatctttcttaaatctttcaaaa

SEQ ID NO: 104 - DNA, 978 bp, Rosa_hybrida_osiana
ttggagagttttgagcttttggagttcggaaaaggccagattgcttccacacaaagttcacttaatacagggagagagagtcatggaagctaacgaatatgaacaacaggagccacttcttcagttctagagaaattatgaacgccgaaactttggtggttgattcgcggtttgtttaggttgtttttgattgagttgaatttagatagtgatagtgatagtgagtgagtgagcgagagcaaagagcaggtttagtgatggaactgttctcgactggtcctccgattaagagaagagcagggctacggattaaacaggcgggtcgggcttcgtaccggggcagttagggagtgttttctttgtgtttttgtggtttttgaatgtgattgattgtgtttgttagtttaatttgagcgtttgagagagttgaaaagagatggggactactgctctgaggcagttattgaagaacctctgcagcaattcgctttggaattatggggtcttttggaagctcaagcatcagaccgatttgattttgagctgggaggatgggtactgtcaccaactgaaaccaagagaaactgtggatcgtgcaacagataatatctactttggtgaggcgaatgagatatcttttaagaagtgtgcaacaggcatacatgaaggtggatctgcggggtatccaattggactagcagtggctgatatgtcacatcttcattacacatttggaaagggggttgtcggtgaggttgcaagtacaggaaaccatagctgggtgctcctcgatggtttgcgcaccagcgtatctgactctaacttagtttcggattgtccagatgagtggttgcttcagtttgcattgggcgtcaagacaattttgcttgtacctgtacttccacatggagttttgcaatttggctccttggaaacggtgcctcattctaaacttaatctaaatgtcttgcattggtt

SEQ ID NO: 105 - DNA, 1459 bp, Rosa_hybrida_osiana
gatctctcgaagagaaacatttgctctctgctccttaatcccccccctccccctcccccaattaacctctcttcctcctcctcctcctcctctctcatctctctataactctccctctctgagactctggggaattaaagacaaaaaccagaaaatgagcaacataagcatggtggaggcaaagttgccccctggttttaggttccatcccagagatgaagagctggtctgtgattatctgatgaagaaggtgactcactccaactccactctcatgatcgaagtcgacctcaacaagtgtgagccttgggacattcctgaaagtgcatgtgtcggaggaaaggaatggtatttctacagtcagcgtgatcggaaatatgcgacggggcttcgaacaaaccgagcaactgcaactgggtattggaaggccaccggaaaagacaggccaatctttcgcaaaggaacaaccctagtgggtatgaggaaaaccttggtgttctaccagggtagggctcccaaaggcagaaaatccgactgggtgatgcatgagttccggcttgaagatccctttggttctcccgaaatatcttcactcaaggaagattgggttctatgcagggtgttttacaaaaacagagaactgattgctgccaaacccagcatgggatcgtcgattagctgctatgacgatgatacaggctcttcgtctcatcagctgccagctctgatggactcttacatcagcttcgatggccaaactcatcaacagcaacagcaacatcaccatgattatgagtatcatcatcatcagcaagtgccctgcttctccattgacacacttttctctcaaaaccaagtgactatcaacccaattttcaccactcaaaacagcaacattatggaacagaacgtaccctccaccggctgtggccagtcgtcgaatgcggcgacaactacattccttgacactgctaatttttcatgtgacaataggaaggtactgaaggctgttttgagtcagcttaccaagatggaaagcagctgctaccctagcacaccgtcgtcggtcttgaaaggctcatcaccacaaagcttcggaggagaagctgctggaagtagttcggataactacctgtctgatcatcaagttggtatgcccaacgtttggagtcactattgattgatatttgctccatttatgtacgtagattcaaacaaatattcaacgaatattgtaaagtcaagtttagaagtatgcatatgcatggaattagcaattagtttgcatgtggtggtttaacttgttttggcttgcgtttcgatgtaagtcattgggtgtattataaatataagggtttgtcttgcttcataattcataaaagaaaacccttaaatttataatatacggatcgagctagtaaagaaaaaaagagacaattagagaaattaatttatttgg

SEQ ID NO: 106 - DNA, 1966 bp, Rosa_hybrida_osiana
tctctccctctctctctctctctggttattgcttctctctcttttaaatttctcttgtgccaaaaatcactctcaaaactgttttcacaacccaagtcttaaaagagagagcacaagagagaacccactcaaaaagaaaaaggctttggctagagatatcaatcccatccctattatagcttcacataacccttttgcttttggttggtgttccatttgttgttttcttcctccatcatctttgtggggttgtgcattgggttctcttatttttatatgggtttcagtctttgagatttttcttcaaacaaactgtctggcttctctttctcataatatcaaggtctttgaggttttgtcttctaagtttggcttcaaaggtatgagctttggtacttgaaagacttgtccaagtgaagttccttcatatctattttctggtgtgaaaagtggagggtaaagaagattttgaatatttattatctgggtcatatgcaaaagatgaaggaggcatgtcctcaacttctggatttgattcccaaagaaagagagtggcttgtgagcaaagatgagagccatggctcttcagaagagaagaaattggagctaaggcttggtcctccaggtcaagactggtcaaatctgagagacaacacctccagaggccacaaagaaagagatgagtctcttctttctcttgggtacttctacaacagcagcagcaatggaaaccaaacccaccattttgcttcctcagagagaaagactgtcctgccctctccatggtcagggtaccaccacaatcagacaaaaggtccttcctttcttcagttctcaccaagctctcagaacctgcctgtcaccaagaaggcgtcacatccttgttgcactaaagcggtagacttgcagagcgcagagaagaagaaagcttctgcaaatccagctgtgaccaacaacacttctcagaaaagaactgctcctgctccagtcgtgggttggcctccaattcgatcttttcggaagaatcttgcgagcaccagctcttcaaaaccagcacctgagtcacaaaatgtagcccctagcaaggttcctaatgttaaaccagtcgaaaccagcggaaaaggcctgtttgtgaaaatcaatatggatggagtccccattggacgaaaagtagacctcagtgcttatgccagctacgagaagctctcctctgctgttgatgaactcttccgtgggctccttgcagctcaaagagattgttgtgctggtggaatcaagaacaagatagaagaagagaaagaaattacaggtttattggatggaagtggagaatatactctcgtatatgaagataatgagggtgacaggatgcttgttggggatgtcccatggcacatgtttgtgtctactgtgaagaggttgcgtgtgctcaagagctctgaactttctgcacttagtattcgcagcggtaaagaactgaagaactgaggagctctactctagagaactgaagtctgtacctaattttgtgttcctacctaaagttttggtattataatttgtcagtgaagaagggaaagagagagacagaagaaagtactagctgcaatcaactaatgcgtacgagtggttcctttttgctgaaaaatcttgtggaaaaagagtaaaaaagacaatgttgtgatggtacattattctaacttagttttgtgtaagaaaccaacttcagccccacccattggtttgggtttataaggtgtcaagccaagcactggttgatctatctgtatgtcattttgattatgcctcctacaaagaaaacctgcgttttgaacttttgattgatttggcacaccagtgacatgtccatcatttgattgagaccactagacggttgcatgttagcagaagatgagagataaaggaaacacgatgaaatcctgcttagatttgatttggccacttt

SEQ ID NO: 107 - DNA, 1978 bp, Rosa_hybrida_osiana
tctctccctctctctctctctctggttattgcttctctctcttttaaatttctcttgtgccaaaaatcactctcaaaactgttttcacaacccaagtcttaaaagagagagcacaagagagaacccactcaaaaagaaaaaggctttggctagagatatcaatcccatccctattatagcttcacataacccttttgcttttggttggtgttccatttgttgttttcttcctccatcatctttgtggggttgtgcattgggttctcttatttttatatgggtttcagtctttgagatttttcttcaaacaaactgtctggcttctctttctcataatatcaaggtctttgaggttttgtcttctaagtttggcttcaaaggtatgagctttggtacttgaaagacttgtccaagtgaagttccttcatatctattttctggtgtgaaaagtggagggtaaagaagattttgaatatttattatctgggtcatatgcaaaagatgaaggaggcatgtcctcaacttctggatttgattcccaaagaaagagagtggcttgtgagcaaagatgagagccatggctcttcagaagagaagaaattggagctaaggcttggtcctccaggtcaagactggtcaaatctgagagacaacacctccagaggccacaaagaaagagatgagtctcttctttctcttgggtacttctacaacagcagcagcaatggaaaccaaacccaccattttgcttcctcagagagaaagactgtcctgccctctccatggtcttcttctccctcagggtaccaccacaaccagacaaaaggtccttcctttcttcagttctcaccaagctctcagagcctgcctgtcacccagaaggcgtcacatccctgttgcactaaagcggtagacttgcagagcgcagagaagaagaaagcttctgcaaatccagctgtgaccaacaacacttctcagaaaagaactgctcctgctccagtcgtgggttggcctccaattcgatcttttcggaagaatcttgcgagcaccagctcttcaaaaccagcacctgagtcacaaaatgtagcccctagcaaggttcctaatgttaaaccagtcgaaaccagcggaaaaggcctgtttgtgaaaatcaatatggatggagtccccattggacgaaaagtagacctcagtgcttatgccagctacgagaagctctcctctgctgttgatgaactcttccgtgggctccttgcagctcaaagagattgttgtgctggtggaatcaagaacaagatagaagaagagaaagaaattacaggtttattggatggaagtggagaatatactctcgtatatgaagataatgagggtgacaggatgcttgttggggatgtcccatggcacatgtttgtgtctactgtgaagaggttgcgtgtgctcaagagctctgaactttctgcacttagtattcgcagcggtaaagaactgaagaactgaggagctctactctagagaactgaagtctgtacctaattttgtgttcctacctaaagttttggtattataatttgtcagtgaagaagggaaagagagagacagaagaaagtactagctgcaatcaactaatgcgtacgagtggttcctttttgctgaaaaatcttgtggaaaaagagtaaaaaagacaatgttgtgatggtacattattctaacttagttttgtgtaagaaaccaacttcagccccacccattggtttgggtttataaggtgtcaagccaagcactggttgatctatctgtatgtcattttgattatgcctcctacaaagaaaacctgcgttttgaacttttgattgatttggcacaccagtgacatgtccatcatttgattgagaccactagacggttgcatgttagcagaagatgagagataaaggaaacacgatgaaatcctgcttagatttgatttggccacttt

SEQ ID NO: 108 - DNA, 1881 bp, Rosa_hybrida_osiana
atgggcgtccagtaccagttagagatgatggtctcaagggtgatgctgggaatctagtcctagaggcatcaaagagtttggaacaaagtattggtggtttgattgatggaggtggaaatactgaagtagaccagcacactcagatggagactaagtcgatcaagccaagacttgtacggatatctgtaacggatggtgatgcaacagagtcctccagcgatgaagaggctgagcccttggccactagtcaccgagtcaagaagttcatcaatgagattatgatcgagtcctcctccacaaaaactgatagtggtgtttggaggagcaggacaacaaggatgtggcggaagaagacaggtggaaaatctggacttccagctacttgccgaacgttgaaggcacagacaacctggaagaagttccgcggtgtgaaggcacaggcaaccaagaacaagttcctaggagtgaggcagagaccatggggaaaatgggcagcagagattagagagcctcatagtggaagacggctctggttgggtacatttgatacggctgaggatgcagctatggtgtacgacaaagcagccgttctgctgcgtggacctgacactttcaccaacttcagtacacctcaggtggcaaatgaagatggcaatttgtaattggaggagttctgtcaagatgattcagaattatgacaacaatttctctacggttaagtatataccgtgcagccaaaaccatggacacagttaggagtttgggactggaaaatcaacggtgaaagtaggcagaccaaagactgcattgcacttgtatgttcaaccatttgatgaatcctttttagcaattttagattttagagtagtgatgtcagattgtcgagatctgaaactttgattgaacagtacataaatgaatatactctgaaaaataccagattctgttttagcaatagtagagactaatgagcctaatctaccttatattggcatcccaattaatagttggcttcatgtttatatatttacacacacacagatacaagagatactatttgcagccaaataagtcaatctacctccatgtttaatacttcaacttccaaatttgtcatcaatgataaggagaagctcttctcatgtgtgctattagtcaatatgcttaaagtattgccagacattgaaggagcagacaatgttgcatcacgctgtgcttcttctttctttttccttttttatttttcttttttcacaggatggtcaagtggagtcgtccataaagatgaaatcattctttatacgtgctgtcgactaccagggtgctgagttttatgtcacgaattcatgatcaagtcgatattggtctcttaattcttcatacgtttgctctcaatgatgttactgccagatccatggtctattggacttgacataaagcacactgatgaatggaagtttctgtttctcctcaattcaggagcaatattatgtgcaactgtctgatctaacttgcctcagaagtagcgcaacagatacagaaagggaaggccaattatttacaaattgccttcaatgtctttcctttccacaggatgatcgaagcagtacttaagggttttggttttctgctgaaaagtggatgttgaattgataggttatctcagaagctttaccctcaaagatgctgaaagtcaggactcatgtcttatgtattattgagtatatgtatatatattgattgtgaattcctttatatatattacatgcacatgtcagagcacatcaattagtaaattccactacaaactatggaatgactgaaatatttacatttctttgtttctgttgttttatggtaaccacaaagttgaaagcgtgg

SEQ ID NO: 109 - DNA, 1194 bp, Rosa_hybrida_osiana
aaccaatattgaccacatcacaaaataaacaccaacaattatgaaatacattaccaaaactaataattgtccaaaacataaataaaatcaaaccaataataattaagcaaaccagttccagttcacaaaaataagcaatccacaaaataaacccaaacaaactgtggtctgcaataattaaacagcccaaattccatgcaagaaaaccaaacaaaaagaaaattatctggaattaaaaaaaaaatacaccaaaagaaaatcaaaccatagattgttcaagggaaacggaatgaagaattgtttggccatgtagcattgtcactgctgtagtctccgaactcccctggcggaagcgttggtagtggatgcaagaagtccatcgtttggggaggtggtgggttcatgatgtccatgaaccagggttgatgctggaacaactgctggtgctggtgctggtgctgcaacctgcatcatctggtactgaatctgctccatagtctgcgcctccatcgcaacttggtgttgttgttgctgcttcacgcctttgatggttccttccccaaatgcactcctcgtcgtcaggtcgagctccattgcagtcgattcaacttccacctggttgagttggttctcctggtttttctcatgcttttgctcattggcaaccagctgcttaattccaagctccctgaggttgcgttcaatgataaatttgaggtcatccagatccatcatgtccatgtcagagatagttctccccttcaaacagccgaacatgacttgagtgatctcattttctcggttgtttcttttttgctttctgatttgcacccaaactttgtcgattctctgtcgtaagaatgtctcctggtcgaccatcattcttatcttctccatttgtggcgtatctcggaacttcgtcagctgttcgtggacttctagatgccttgggaacacctctagctcggcgtcaaaggggttgtagatgatcaccatggccttgatgtcacagagagtggttatctctcccaccttcttcagaagacccttctttctttttctaaatgtcgttcgtcgggaactctcattggtaatatactacagtctcaccttccttctagccatggctaactagctagcaaaggaaaaggtcagtagctggggatcgaactagggtataaagaaagagatttg

SEQ ID NO: 110 - DNA, 1111 bp, Rosa_hybrida_osiana
gacataaagagaggaagacaagagagatagagatacggatagagagagggaaatggggaggagtccatgctgctccaaggaaggacttaacagaggagcttggactgccttggaagataaagtactaacgtcttacatcaaagcccatggagaaggcaaatggagaaacctcccaaagagagctggtttgaagagatgtggtaagagctgtagactgagatggttaaactatctgaggccagacataaagagaggtaatatatccggggatgaagaagaactcattatcagactccataatcttcttggcaacagatggtctctaatagctggaaggctaccggggcgaacagacaatgaaatcaaaaattactggaacaccactctggtgaagaaagctaaacccgaatcgcattcgggatcatccaaggaaacatctcccgctccgaccaaattcaggcccagaacgccatcagccacagccactcaacctcaagtaataagaaccaaggccactaggttaaccagaatgctagtcccatcactcccacttctaatcgatgattactcaacaaccacaagcccattagatcttcgagttcctcaaacccagtcggtaagttcacttcctgaagacgcagcaaatacagaagtacacattcaggggacagatgcaatgaactttggctgcaatggatttcaagcagctgctggtgaggatgaagatgctatgggaggctacgatattccattagatgatggaatgctcaatgattggacaggaaatgataactgtgatcttgagaaatatggtgcttcattggatttagattccttggcatttttgctggactctgatgactggctgggccaagaaaatagtatctaatctttaaatttatggtaaataggttataatgtcacacaagttactaatatgtaaacaataaaagtgaggctaaacaaaatcaaaaatcggtacgtacgattcttattcctctgtaattgagatgggtttttgaatccgagagattttattctagaaattaacattgtcacaaaaaatatcacttaatagtgagctcgtattttaatcttcaaatttatatgt

SEQ ID NO: 111 - DNA, 1703 bp, Rosa_hybrida_osiana
aagcagtgatgttgttgttttaagattcattcattcaacacaagctaaagaaagagaagttgaacagaactacattattataacaaatacagctacattagctattataatagccccatattttacaaaccaagaacacaataccacaaactttgttttttgcacattaaattattccttaacaccatctaattaacagtgagattcctcccaccatagactaattaagctgaaatcaaaagtacacctctctggctctctctctgtgtccctctttcttaatccagaatccaaagaattaatcccctggagatcagtactgaaacatctctgcagaagccatgatgggatggttcagatatgaaagattcaaagggttgaagaagcaatcagcactaggctcagtgaagttgttgttgcttgaatcctcaacaaaaccaccatggtttgcattcaagaaagcgatgatctcattcagagactgcagcctgttgctgagctcagacacttgggctctgagcacagagttctcagcctcaatgttcatgtaatgctggcttgtgatgttgaggctgctgatgatctggtggttctccttcctcagctgagccatctgcgccgtcagatcatccaagtgcttctgtttcctcatcctagaccttcttgcagattcacgattcgacaacattctctttctttttctctgatccatcagagcctgcaagtcttcttcagagccagagttttgaatcatggaggaaacagaggatgttccactagaagaagccattggaaacccagaaacttttttgcagatttcgacagtaattaagagtaactcagacagataattaggtaaaaacagtacccagatgcagcataaacttaaatattgcagataaaatagtaaataatagctataatataggttgtttggggcctattatcagaatttgggacctatgaaacgtagtaaagccagtagaggaagacaactgagaatgactgaactaaatgggtcctgcgcctatatgtggaagatatcatacactcggatagaaggagttcactcagagttggagacatgaatctcaaacatcaagaacaagaggttaagcatcaagataaatttcttgaaaacctaaacaacaagccctgagaaagtctgtgaactgggtatttgaaaaggctcaaaaaacccaaaccagcataataaagtctggacctttctgagatttgaggtcaagaaaatgggatcttgacagaagaaaaaaaaacaaacccaaaaacggcaggatttcaagatgatcttgaaccaagaaaccagaaggaacacaagatctcagagcaaaaacaagagcaaggaagctacaggttaaagctggagccttaaggaaacaactatggaccaaagggagaagagggctttgaggggctttgagaagggaatttatagagaaaaaggagagcaaatggtgatcataaacagaaatattaaaattggggagaaatgagaaaaacaaagagacgaaaaagactttggaatcaaatcgtatagaatagtggcgagacatgtgaggtgaggaaagagaggacctcactccacagccgtccagatccagctgggaaccaacaatctcatgttaatttttttgttttatagtgatgggtagggaggaactgaggaagtacaaagggcatta